SEQUENCE LISTING (147 sequences)

SEQ ID NO: 1 - protein, 396 aa, synthetic construct
METDTLLLWVLLLWVPGSTGEIVMTQSPATLSVSPGERATLSCKASQSVSNDVVWYQQKPGQAPRLLIYYASNRYTGIPARFSGSGSGTEFTLTISSLQSEDFAVYYCQQDYTSPWTFGQGTKLEIKGSTSGGGSGGGSGGGGSSQVQLVQSGAEVKKPGASVKVSCKASGYTFTNYGMNWVRQAPGQGLEWMGWINTYTGEPTYADDFKGRVTMTTDTSTSTAYMELRSLRSDDTAVYYCARIGDSSPSDYWGQGTLVTVSSEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 396 aa, synthetic construct
METDTLLLWVLLLWVPGSTGQVQLVQSGAEVKKPGASVKVSCKASGYTFTNYGMNWVRQAPGQGLEWMGWINTYTGEPTYADDFKGRVTMTTDTSTSTAYMELRSLRSDDTAVYYCARIGDSSPSDYWGQGTLVTVSSGSTSGGGSGGGSGGGGSSEIVMTQSPATLSVSPGERATLSCKASQSVSNDVVWYQQKPGQAPRLLIYYASNRYTGIPARFSGSGSGTEFTLTISSLQSEDFAVYYCQQDYTSPWTFGQGTKLEIKEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 396 aa, synthetic construct
METDTLLLWVLLLWVPGSTGEIVMTQSPATLSVSPGERATLSCKASQSVSNDVVWYQQKPGQAPRLLIYDASNRYTGIPARFSGSGSGTEFTLTISSLQSEDFAVYYCQQDYTSPWTFGQGTKLEIKGSTSGGGSGGGSGGGGSSQVQLVQSGAEVKKPGASVKVSCKASGYTFTNYGMNWVRQAPGQGLEWMGWINTYTGEPTYADDFKGRVTMTTDTSTSTAYMELRSLRSDDTAVYYCARIGDSSPSDYWGQGTLVTVSSEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 4 - protein, 396 aa, synthetic construct
METDTLLLWVLLLWVPGSTGQVQLVQSGAEVKKPGASVKVSCKASGYTFTNYGMNWVRQAPGQGLEWMGWINTYTGEPTYADDFKGRVTMTTDTSTSTAYMELRSLRSDDTAVYYCARIGDSSPSDYWGQGTLVTVSSGSTSGGGSGGGSGGGGSSEIVMTQSPATLSVSPGERATLSCKASQSVSNDVVWYQQKPGQAPRLLIYDASNRYTGIPARFSGSGSGTEFTLTISSLQSEDFAVYYCQQDYTSPWTFGQGTKLEIKEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 5 - protein, 396 aa, synthetic construct
METDTLLLWVLLLWVPGSTGEIVMTQSPATLSVSPGERATLSCKASQSVSNDVVWYQQKPGQAPRLLIYYASNRNTGIPARFSGSGSGTEFTLTISSLQSEDFAVYYCQQDYTSPWTFGQGTKLEIKGSTSGGGSGGGSGGGGSSQVQLVQSGAEVKKPGASVKVSCKASGYTFTNYGMNWVRQAPGQGLEWMGWINTYTGEPTYADDFKGRVTMTTDTSTSTAYMELRSLRSDDTAVYYCARIGDSSPSDYWGQGTLVTVSSEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - protein, 396 aa, synthetic construct
METDTLLLWVLLLWVPGSTGQVQLVQSGAEVKKPGASVKVSCKASGYTFTNYGMNWVRQAPGQGLEWMGWINTYTGEPTYADDFKGRVTMTTDTSTSTAYMELRSLRSDDTAVYYCARIGDSSPSDYWGQGTLVTVSSGSTSGGGSGGGSGGGGSSEIVMTQSPATLSVSPGERATLSCKASQSVSNDVVWYQQKPGQAPRLLIYYASNRNTGIPARFSGSGSGTEFTLTISSLQSEDFAVYYCQQDYTSPWTFGQGTKLEIKEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 7 - protein, 396 aa, synthetic construct
METDTLLLWVLLLWVPGSTGEIVMTQSPATLSVSPGERATLSCKASQSVSNDVVWYQQKPGQAPRLLIYDASNRNTGIPARFSGSGSGTEFTLTISSLQSEDFAVYYCQQDYTSPWTFGQGTKLEIKGSTSGGGSGGGSGGGGSSQVQLVQSGAEVKKPGASVKVSCKASGYTFTNYGMNWVRQAPGQGLEWMGWINTYTGEPTYADDFKGRVTMTTDTSTSTAYMELRSLRSDDTAVYYCARIGDSSPSDYWGQGTLVTVSSEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 8 - protein, 396 aa, synthetic construct
METDTLLLWVLLLWVPGSTGQVQLVQSGAEVKKPGASVKVSCKASGYTFTNYGMNWVRQAPGQGLEWMGWINTYTGEPTYADDFKGRVTMTTDTSTSTAYMELRSLRSDDTAVYYCARIGDSSPSDYWGQGTLVTVSSGSTSGGGSGGGSGGGGSSEIVMTQSPATLSVSPGERATLSCKASQSVSNDVVWYQQKPGQAPRLLIYDASNRNTGIPARFSGSGSGTEFTLTISSLQSEDFAVYYCQQDYTSPWTFGQGTKLEIKEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 9 - protein, 396 aa, synthetic construct
METDTLLLWVLLLWVPGSTGEIVMTQSPATLSVSPGERATLSCKASQSVSNDVVWYQQKPGQAPQLLIYDASNRYTGIPARFSGSGSGTEFTLTISSLQSEDFAVYYCQQDYTSPWTFGQGTKLEIKGSTSGGGSGGGSGGGGSSQVQLVQSGAEVKKPGASVKVSCKASGYTFTNYGMNWVRQAPGQGLEWMGWINTYTGEPTYADDFKGRVTMTTDTSTSTAYMELRSLRSDDTAVYYCARIGDSSPSDYWGQGTLVTVSSEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 396 aa, synthetic construct
METDTLLLWVLLLWVPGSTGEIVMTQSPATLSVSPGERATLSCKASQSVSNDVVWYQQKPGQSPQLLIYDASNRYTGIPARFSGSGSGTEFTLTISSLQSEDFAVYYCQQDYTSPWTFGQGTKLEIKGSTSGGGSGGGSGGGGSSQVQLVQSGAEVKKPGASVKVSCKASGYTFTNYGMNWVRQAPGQGLEWMGWINTYTGEPTYADDFKGRVTMTTDTSTSTAYMELRSLRSDDTAVYYCARIGDSSPSDYWGQGTLVTVSSEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 259 aa, synthetic construct
METDTLLLWVLLLWVPGSTGEIVMTQSPATLSVSPGERATLSCKASQSVSNDVVWYQQKPGQAPRLLIYDASNRYTGIPARFSGSGSGTEFTLTISSLQSEDFAVYYCQQDYTSPWTFGQGTKLEIKSGGGGQVQLVQSGAEVKKPGASVKVSCKASGYTFTNYGMNWVRQAPGQGLEWMGWINTYTGEPTYADDFKGRVTMTTDTSTSTAYMELRSLRSDDTAVYYCARIGDSSPSDYWGQGTLVTVSSGGCPPCPPC

SEQ ID NO: 12 - protein, 259 aa, synthetic construct
METDTLLLWVLLLWVPGSTGQVQLVQSGAEVKKPGASVKVSCKASGYTFTNYGMNWVRQAPGQGLEWMGWINTYTGEPTYADDFKGRVTMTTDTSTSTAYMELRSLRSDDTAVYYCARIGDSSPSDYWGQGTLVTVSSSGGGGEIVMTQSPATLSVSPGERATLSCKASQSVSNDVVWYQQKPGQAPRLLIYDASNRYTGIPARFSGSGSGTEFTLTISSLQSEDFAVYYCQQDYTSPWTFGQGTKLEIKGGCPPCPPC

SEQ ID NO: 13 - protein, 262 aa, synthetic construct
METDTLLLWVLLLWVPGSTGEIVMTQSPATLSVSPGERATLSCKASQSVSNDVVWYQQKPGQAPRLLIYDASNRYTGIPARFSGSGSGTEFTLTISSLQSEDFAVYYCQQDYTSPWTFGQGTKLEIKGGGSGGGGQVQLVQSGAEVKKPGASVKVSCKASGYTFTNYGMNWVRQAPGQGLEWMGWINTYTGEPTYADDFKGRVTMTTDTSTSTAYMELRSLRSDDTAVYYCARIGDSSPSDYWGQGTLVTVSSGGCPPCPPC

SEQ ID NO: 14 - protein, 262 aa, synthetic construct
METDTLLLWVLLLWVPGSTGQVQLVQSGAEVKKPGASVKVSCKASGYTFTNYGMNWVRQAPGQGLEWMGWINTYTGEPTYADDFKGRVTMTTDTSTSTAYMELRSLRSDDTAVYYCARIGDSSPSDYWGQGTLVTVSSGGGSGGGGEIVMTQSPATLSVSPGERATLSCKASQSVSNDVVWYQQKPGQAPRLLIYDASNRYTGIPARFSGSGSGTEFTLTISSLQSEDFAVYYCQQDYTSPWTFGQGTKLEIKGGCPPCPPC

SEQ ID NO: 15 - protein, 12 aa, synthetic construct
KASQSVSNDVVW

SEQ ID NO: 16 - protein, 36 aa, synthetic construct
LYSALASERGLNSERVALSERASNASPVALVALTRP

SEQ ID NO: 17 - protein, 8 aa, synthetic construct
YASNRYTG

SEQ ID NO: 18 - protein, 24 aa, synthetic construct
TYRALASERASNARGTYRTHRGLY

SEQ ID NO: 19 - protein, 8 aa, synthetic construct
DASNRYTG

SEQ ID NO: 20 - protein, 24 aa, synthetic construct
ASPALASERASNARGTYRTHRGLY

SEQ ID NO: 21 - protein, 8 aa, synthetic construct
YASNRNTG

SEQ ID NO: 22 - protein, 24 aa, synthetic construct
TYRALASERASNARGASNTHRGLY

SEQ ID NO: 23 - protein, 8 aa, synthetic construct
DASNRNTG

SEQ ID NO: 24 - protein, 24 aa, synthetic construct
ASPALASERASNARGASNTHRGLY

SEQ ID NO: 25 - protein, 9 aa, synthetic construct
QQDYTSPWT

SEQ ID NO: 26 - protein, 27 aa, synthetic construct
GLNGLNASPTYRTHRSERPROTRPTHR

SEQ ID NO: 27 - protein, 13 aa, synthetic construct
KASGYTFTNYGMN

SEQ ID NO: 28 - protein, 39 aa, synthetic construct
LYSALASERGLYTYRTHRPHETHRASNTYRGLYMETASN

SEQ ID NO: 29 - protein, 10 aa, synthetic construct
WINTYTGEPT

SEQ ID NO: 30 - protein, 30 aa, synthetic construct
TRPILEASNTHRTYRTHRGLYGLUPROTHR

SEQ ID NO: 31 - protein, 11 aa, synthetic construct
ARIGDSSPSDY

SEQ ID NO: 32 - protein, 57 aa, synthetic construct
ALAARGILEGLYASPSERSERPROSERASPTYRASPALASERASNARGASNTHRGLY

SEQ ID NO: 33 - protein, 14 aa, synthetic construct
YQQKPGQAPRLLIY

SEQ ID NO: 34 - protein, 42 aa, synthetic construct
TYRGLNGLNLYSPROGLYGLNALAPROARGLEULEUILETYR

SEQ ID NO: 35 - protein, 14 aa, synthetic construct
YQQKPGQAPQLLIY

SEQ ID NO: 36 - protein, 42 aa, synthetic construct
TYRGLNGLNLYSPROGLYGLNALAPROGLNLEULEUILETYR

SEQ ID NO: 37 - protein, 14 aa, synthetic construct
YQQKPGQSPQLLIY

SEQ ID NO: 38 - protein, 42 aa, synthetic construct
TYRGLNGLNLYSPROGLYGLNSERPROGLNLEULEUILETYR

SEQ ID NO: 39 - protein, 5 aa, synthetic construct
SGGGG

SEQ ID NO: 40 - protein, 15 aa, synthetic construct
SERGLYGLYGLYGLY

SEQ ID NO: 41 - protein, 8 aa, synthetic construct
GGGSGGGG

SEQ ID NO: 42 - protein, 24 aa, synthetic construct
GLYGLYGLYSERGLYGLYGLYGLY

SEQ ID NO: 43 - protein, 18 aa, synthetic construct
GSTSGGGSGGGSGGGGSS

SEQ ID NO: 44 - protein, 54 aa, synthetic construct
GLYSERTHRSERGLYGLYGLYSERGLYGLYGLYSERGLYGLYGLYGLYSERSER

SEQ ID NO: 45 - protein, 20 aa, synthetic construct
METDTLLLWVLLLWVPGSTG

SEQ ID NO: 46 - protein, 60 aa, synthetic construct
METGLUTHRASPTHRLEULEULEUTRPVALLEULEULEUTRPVALPROGLYSERTHRGLY

SEQ ID NO: 47 - protein, 447 aa, synthetic construct
QVQLVQSGAEVKKPGASVKVSCKASGYTFTNYGMNWVRQAPGQGLEWMGWINTYTGEPTYADDFKGRVTMTTDTSTSTAYMELRSLRSDDTAVYYCARIGDSSPSDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 48 - protein, 214 aa, synthetic construct
EIVMTQSPATLSVSPGERATLSCKASQSVSNDVVWYQQKPGQAPRLLIYYASNRYTGIPARFSGSGSGTEFTLTISSLQSEDFAVYYCQQDYTSPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 107 aa, synthetic construct
EIVMTQSPATLSVSPGERATLSCKASQSVSNDVVWYQQKPGQAPRLLIYYASNRYTGIPARFSGSGSGTEFTLTISSLQSEDFAVYYCQQDYTSPWTFGQGTKLEIK

SEQ ID NO: 50 - protein, 618 aa, Homo sapiens
MVSPRMSGLLSQTVILALIFLPQTRPAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLLPPALGLLVAAGVAGAALLLVHVRRRGHSQDAGSRLLAGTPEPSVHALPDALNNLRTQEGSGDGPSSSVDWNRPEDVDPQGIYVISAPSIYAREVATPLFPPLHTGRAGQRQHLLFPYPSSILSVK

SEQ ID NO: 51 - DNA, 1191 bp, synthetic construct
  source 1..1191: mol_type = other DNA
atggagaccgacaccctgctgctgtgggtgctcctactgtgggtgcccggcagcaccggcgagatcgtgatgacacagagccccgcgacactcagcgtgagtcccggcgagagagcgacgctcagctgcaaggcaagccaaagcgtgagcaacgacgtggtgtggtatcagcagaagcccggccaagcccctagactgctgatctactacgctagcaacagatacaccggcatccccgctagattcagcggcagcggcagcggcaccgagttcaccctgaccatcagcagcctgcagagcgaggacttcgccgtttactactgtcaacaagactacacaagcccctggaccttcggccaaggcaccaagctggaaattaagggcagcacctctggaggcgggtcgggcggaggatcgggaggcggcggaagcagccaagtgcagctggtgcagagcggcgccgaggtgaagaagcccggcgctagcgtgaaggtatcttgcaaggctagcgggtataccttcaccaactacggcatgaactgggtgagacaagcccccggccaaggcctggagtggatgggctggatcaacacctacaccggcgagcccacctacgccgacgacttcaagggcagagtgaccatgaccaccgacacaagcacaagcaccgcctacatggagctgagaagcctgagaagcgacgacaccgctgtgtattattgcgcgcggatcggcgacagcagccctagcgactactggggacaaggcaccctggttaccgtgagcagcgaacccaagagctccgacaagacccacacgtgtcccccttgccctccttgtggcggaggaagctccggaggcggaagcggaggacagcctagggagccccaggtgtataccctccccccctccagggaagagatgaccaagaaccaggtgagcctgacctgcctcgtgaagggcttttatccctccgatatcgccgtggagtgggagagcaacggccagcctgagaacaattacaagaccaccccccctgtgctggactccgatggcagcttcttcctgtattccaagctgaccgtcgacaagtccaggtggcaacagggcaacgtcttcagctgcagcgtgatgcacgaggccctgcacaatcactacacccagaagtccctctccctgagccccggctga

SEQ ID NO: 52 - DNA, 1191 bp, synthetic construct
  source 1..1191: mol_type = other DNA
atggagaccgataccctgctactgtgggtcctcctactgtgggtgcccggcagcaccgggcaagtgcagctggtgcagagcggcgccgaggtgaagaagcccggcgctagcgtgaaggtgagctgcaaggcaagcggttacaccttcaccaactacggcatgaactgggtgagacaagcccccggccaaggcctggagtggatgggctggatcaacacctacaccggcgagcccacctacgccgacgacttcaagggcagagtgaccatgaccaccgacacaagcacaagcaccgcctacatggagctgagaagcctgagaagcgacgacaccgccgtttactactgtgcgagaatcggcgacagcagccctagcgactattggggtcaaggcaccttggttactgtgtcaagcggctccacctccggcggcggatcgggcggaggaagcggtggcggcggcagcagcgagatcgtgatgacacagagccccgccaccttaagcgttagccccggcgagagagcaaccctttcttgcaaagctagccaaagtgtgagcaacgacgtggtgtggtatcagcagaagcccggccaagcccctagactgctgatctactacgctagcaacagatacaccggcatccccgctagattcagcggcagcggcagcggcaccgagttcaccctgaccatcagcagcctgcagagcgaggacttcgctgtgtattattgccaacaagactacacaagcccctggaccttcggtcaagggaccaagctggagatcaaggaacccaagagctccgacaagacccacacgtgtcccccttgccctccttgtggcggaggaagctccggaggcggaagcggaggacagcctagggagccccaggtgtataccctccccccctccagggaagagatgaccaagaaccaggtgagcctgacctgcctcgtgaagggcttttatccctccgatatcgccgtggagtgggagagcaacggccagcctgagaacaattacaagaccaccccccctgtgctggactccgatggcagcttcttcctgtattccaagctgaccgtcgacaagtccaggtggcaacagggcaacgtcttcagctgcagcgtgatgcacgaggccctgcacaatcactacacccagaagtccctctccctgagccccggctga

SEQ ID NO: 53 - DNA, 1191 bp, synthetic construct
  source 1..1191: mol_type = other DNA
atggagaccgacaccctgctgctgtgggtgctcctactgtgggtgcccggcagcaccggcgagatcgtgatgacacagagccccgcgacactcagcgtgagtcccggcgagagagcgacgctcagctgcaaggcaagccaaagcgtgagcaacgacgtggtgtggtatcagcagaagcccggccaagcccctagactgctgatctacgacgctagcaacagatacaccggcatccccgctagattcagcggcagcggcagcggcaccgagttcaccctgaccatcagcagcctgcagagcgaggacttcgccgtttactactgtcaacaagactacacaagcccctggaccttcggccaaggcaccaagctggaaattaagggcagcacctctggaggcgggtcgggcggaggatcgggaggcggcggaagcagccaagtgcagctggtgcagagcggcgccgaggtgaagaagcccggcgctagcgtgaaggtatcttgcaaggctagcgggtataccttcaccaactacggcatgaactgggtgagacaagcccccggccaaggcctggagtggatgggctggatcaacacctacaccggcgagcccacctacgccgacgacttcaagggcagagtgaccatgaccaccgacacaagcacaagcaccgcctacatggagctgagaagcctgagaagcgacgacaccgctgtgtattattgcgcgcggatcggcgacagcagccctagcgactactggggacaaggcaccctggttaccgtgagcagcgaacccaagagctccgacaagacccacacgtgtcccccttgccctccttgtggcggaggaagctccggaggcggaagcggaggacagcctagggagccccaggtgtataccctccccccctccagggaagagatgaccaagaaccaggtgagcctgacctgcctcgtgaagggcttttatccctccgatatcgccgtggagtgggagagcaacggccagcctgagaacaattacaagaccaccccccctgtgctggactccgatggcagcttcttcctgtattccaagctgaccgtcgacaagtccaggtggcaacagggcaacgtcttcagctgcagcgtgatgcacgaggccctgcacaatcactacacccagaagtccctctccctgagccccggctga

SEQ ID NO: 54 - DNA, 1191 bp, synthetic construct
  source 1..1191: mol_type = other DNA
atggagaccgataccctgctactgtgggtcctcctactgtgggtgcccggcagcaccgggcaagtgcagctggtgcagagcggcgccgaggtgaagaagcccggcgctagcgtgaaggtgagctgcaaggcaagcggttacaccttcaccaactacggcatgaactgggtgagacaagcccccggccaaggcctggagtggatgggctggatcaacacctacaccggcgagcccacctacgccgacgacttcaagggcagagtgaccatgaccaccgacacaagcacaagcaccgcctacatggagctgagaagcctgagaagcgacgacaccgccgtttactactgtgcgagaatcggcgacagcagccctagcgactattggggtcaaggcaccttggttactgtgtcaagcggctccacctccggcggcggatcgggcggaggaagcggtggcggcggcagcagcgagatcgtgatgacacagagccccgccaccttaagcgttagccccggcgagagagcaaccctttcttgcaaagctagccaaagtgtgagcaacgacgtggtgtggtatcagcagaagcccggccaagcccctagactgctgatctacgacgctagcaacagatacaccggcatccccgctagattcagcggcagcggcagcggcaccgagttcaccctgaccatcagcagcctgcagagcgaggacttcgctgtgtattattgccaacaagactacacaagcccctggaccttcggtcaagggaccaagctggagatcaaggaacccaagagctccgacaagacccacacgtgtcccccttgccctccttgtggcggaggaagctccggaggcggaagcggaggacagcctagggagccccaggtgtataccctccccccctccagggaagagatgaccaagaaccaggtgagcctgacctgcctcgtgaagggcttttatccctccgatatcgccgtggagtgggagagcaacggccagcctgagaacaattacaagaccaccccccctgtgctggactccgatggcagcttcttcctgtattccaagctgaccgtcgacaagtccaggtggcaacagggcaacgtcttcagctgcagcgtgatgcacgaggccctgcacaatcactacacccagaagtccctctccctgagccccggctga

SEQ ID NO: 55 - DNA, 1191 bp, synthetic construct
  source 1..1191: mol_type = other DNA
atggagaccgacaccctgctgctgtgggtgctcctactgtgggtgcccggcagcaccggcgagatcgtgatgacacagagccccgcgacactcagcgtgagtcccggcgagagagcgacgctcagctgcaaggcaagccaaagcgtgagcaacgacgtggtgtggtatcagcagaagcccggccaagcccctagactgctgatctactacgctagcaacagaaacaccggcatccccgctagattcagcggcagcggcagcggcaccgagttcaccctgaccatcagcagcctgcagagcgaggacttcgccgtttactactgtcaacaagactacacaagcccctggaccttcggccaaggcaccaagctggaaattaagggcagcacctctggaggcgggtcgggcggaggatcgggaggcggcggaagcagccaagtgcagctggtgcagagcggcgccgaggtgaagaagcccggcgctagcgtgaaggtatcttgcaaggctagcgggtataccttcaccaactacggcatgaactgggtgagacaagcccccggccaaggcctggagtggatgggctggatcaacacctacaccggcgagcccacctacgccgacgacttcaagggcagagtgaccatgaccaccgacacaagcacaagcaccgcctacatggagctgagaagcctgagaagcgacgacaccgctgtgtattattgcgcgcggatcggcgacagcagccctagcgactactggggacaaggcaccctggttaccgtgagcagcgaacccaagagctccgacaagacccacacgtgtcccccttgccctccttgtggcggaggaagctccggaggcggaagcggaggacagcctagggagccccaggtgtataccctccccccctccagggaagagatgaccaagaaccaggtgagcctgacctgcctcgtgaagggcttttatccctccgatatcgccgtggagtgggagagcaacggccagcctgagaacaattacaagaccaccccccctgtgctggactccgatggcagcttcttcctgtattccaagctgaccgtcgacaagtccaggtggcaacagggcaacgtcttcagctgcagcgtgatgcacgaggccctgcacaatcactacacccagaagtccctctccctgagccccggctga

SEQ ID NO: 56 - DNA, 1191 bp, synthetic construct
  source 1..1191: mol_type = other DNA
atggagaccgataccctgctactgtgggtcctcctactgtgggtgcccggcagcaccgggcaagtgcagctggtgcagagcggcgccgaggtgaagaagcccggcgctagcgtgaaggtgagctgcaaggcaagcggttacaccttcaccaactacggcatgaactgggtgagacaagcccccggccaaggcctggagtggatgggctggatcaacacctacaccggcgagcccacctacgccgacgacttcaagggcagagtgaccatgaccaccgacacaagcacaagcaccgcctacatggagctgagaagcctgagaagcgacgacaccgccgtttactactgtgcgagaatcggcgacagcagccctagcgactattggggtcaaggcaccttggttactgtgtcaagcggctccacctccggcggcggatcgggcggaggaagcggtggcggcggcagcagcgagatcgtgatgacacagagccccgccaccttaagcgttagccccggcgagagagcaaccctttcttgcaaagctagccaaagtgtgagcaacgacgtggtgtggtatcagcagaagcccggccaagcccctagactgctgatctactacgctagcaacagaaacaccggcatccccgctagattcagcggcagcggcagcggcaccgagttcaccctgaccatcagcagcctgcagagcgaggacttcgctgtgtattattgccaacaagactacacaagcccctggaccttcggtcaagggaccaagctggagatcaaggaacccaagagctccgacaagacccacacgtgtcccccttgccctccttgtggcggaggaagctccggaggcggaagcggaggacagcctagggagccccaggtgtataccctccccccctccagggaagagatgaccaagaaccaggtgagcctgacctgcctcgtgaagggcttttatccctccgatatcgccgtggagtgggagagcaacggccagcctgagaacaattacaagaccaccccccctgtgctggactccgatggcagcttcttcctgtattccaagctgaccgtcgacaagtccaggtggcaacagggcaacgtcttcagctgcagcgtgatgcacgaggccctgcacaatcactacacccagaagtccctctccctgagccccggctga

SEQ ID NO: 57 - DNA, 1191 bp, synthetic construct
  source 1..1191: mol_type = other DNA
atggagaccgacaccctgctgctgtgggtgctcctactgtgggtgcccggcagcaccggcgagatcgtgatgacacagagccccgcgacactcagcgtgagtcccggcgagagagcgacgctcagctgcaaggcaagccaaagcgtgagcaacgacgtggtgtggtatcagcagaagcccggccaagcccctagactgctgatctacgacgctagcaacagaaacaccggcatccccgctagattcagcggcagcggcagcggcaccgagttcaccctgaccatcagcagcctgcagagcgaggacttcgccgtttactactgtcaacaagactacacaagcccctggaccttcggccaaggcaccaagctggaaattaagggcagcacctctggaggcgggtcgggcggaggatcgggaggcggcggaagcagccaagtgcagctggtgcagagcggcgccgaggtgaagaagcccggcgctagcgtgaaggtatcttgcaaggctagcgggtataccttcaccaactacggcatgaactgggtgagacaagcccccggccaaggcctggagtggatgggctggatcaacacctacaccggcgagcccacctacgccgacgacttcaagggcagagtgaccatgaccaccgacacaagcacaagcaccgcctacatggagctgagaagcctgagaagcgacgacaccgctgtgtattattgcgcgcggatcggcgacagcagccctagcgactactggggacaaggcaccctggttaccgtgagcagcgaacccaagagctccgacaagacccacacgtgtcccccttgccctccttgtggcggaggaagctccggaggcggaagcggaggacagcctagggagccccaggtgtataccctccccccctccagggaagagatgaccaagaaccaggtgagcctgacctgcctcgtgaagggcttttatccctccgatatcgccgtggagtgggagagcaacggccagcctgagaacaattacaagaccaccccccctgtgctggactccgatggcagcttcttcctgtattccaagctgaccgtcgacaagtccaggtggcaacagggcaacgtcttcagctgcagcgtgatgcacgaggccctgcacaatcactacacccagaagtccctctccctgagccccggctga

SEQ ID NO: 58 - DNA, 1191 bp, synthetic construct
  source 1..1191: mol_type = other DNA
atggagaccgataccctgctactgtgggtcctcctactgtgggtgcccggcagcaccgggcaagtgcagctggtgcagagcggcgccgaggtgaagaagcccggcgctagcgtgaaggtgagctgcaaggcaagcggttacaccttcaccaactacggcatgaactgggtgagacaagcccccggccaaggcctggagtggatgggctggatcaacacctacaccggcgagcccacctacgccgacgacttcaagggcagagtgaccatgaccaccgacacaagcacaagcaccgcctacatggagctgagaagcctgagaagcgacgacaccgccgtttactactgtgcgagaatcggcgacagcagccctagcgactattggggtcaaggcaccttggttactgtgtcaagcggctccacctccggcggcggatcgggcggaggaagcggtggcggcggcagcagcgagatcgtgatgacacagagccccgccaccttaagcgttagccccggcgagagagcaaccctttcttgcaaagctagccaaagtgtgagcaacgacgtggtgtggtatcagcagaagcccggccaagcccctagactgctgatctacgacgctagcaacagaaacaccggcatccccgctagattcagcggcagcggcagcggcaccgagttcaccctgaccatcagcagcctgcagagcgaggacttcgctgtgtattattgccaacaagactacacaagcccctggaccttcggtcaagggaccaagctggagatcaaggaacccaagagctccgacaagacccacacgtgtcccccttgccctccttgtggcggaggaagctccggaggcggaagcggaggacagcctagggagccccaggtgtataccctccccccctccagggaagagatgaccaagaaccaggtgagcctgacctgcctcgtgaagggcttttatccctccgatatcgccgtggagtgggagagcaacggccagcctgagaacaattacaagaccaccccccctgtgctggactccgatggcagcttcttcctgtattccaagctgaccgtcgacaagtccaggtggcaacagggcaacgtcttcagctgcagcgtgatgcacgaggccctgcacaatcactacacccagaagtccctctccctgagccccggctga

SEQ ID NO: 59 - DNA, 780 bp, synthetic construct
  source 1..780: mol_type = other DNA
atggagaccgacaccctgctgctgtgggtgctgctcctgtgggtgcccggtagcaccggcgagatcgtgatgacacagagccccgcgaccctcagcgtcagtcccggcgagagagccacgctgagctgcaaggcatctcagagcgtgagcaacgacgtggtgtggtatcagcagaagcccggccaagcccctagactgctgatctacgacgctagcaacagatacaccggcatccccgctagattcagcggcagcggcagcggcaccgagttcaccctgaccatcagcagcctgcagagcgaggacttcgctgtgtactactgccaacaagactacacaagcccctggaccttcgggcaaggcaccaaactggaaattaagagcggaggtggtgggcaagtgcagctggtgcagagcggcgccgaggtgaagaagcccggcgctagcgtgaaagtgagctgcaaggcctccggatacaccttcaccaactacggcatgaactgggtgagacaagcccccggccaaggcctggagtggatgggctggatcaacacctacaccggcgagcccacctacgccgacgacttcaagggcagagtgaccatgaccaccgacacaagcacaagcaccgcctacatggagctgagaagcctgagaagcgacgacaccgccgtgtactattgcgcgcggatcggcgacagcagccctagcgactactggggccaaggcaccctggttaccgtgagcagcggcggctgccccccctgcccaccctgctga

SEQ ID NO: 60 - DNA, 780 bp, synthetic construct
  source 1..780: mol_type = other DNA
atggagaccgacaccctgctgctgtgggtgctcctgctgtgggttccgggatccaccggccaagtgcagctggtgcagagcggcgccgaggtgaagaagcccggcgctagcgtgaaggtaagctgcaaggctagcgggtacaccttcaccaactacggcatgaactgggtgagacaagcccccggccaaggcctggagtggatgggctggatcaacacctacaccggcgagcccacctacgccgacgacttcaagggcagagtgaccatgaccaccgacacaagcacaagcaccgcctacatggagctgagaagcctgagaagcgacgacaccgctgtgtactactgcgctcgcatcggcgacagcagccctagcgactactgggggcaaggcacccttgtcaccgtgagctcgagcggtggcggcggtgagatcgtgatgacacagagccccgctacactgagcgtgagccccggcgagagagccacgctgagctgcaaagcctctcagagcgtgagcaacgacgtggtgtggtatcagcagaagcccggccaagcccctagactgctgatctacgacgctagcaacagatacaccggcatccccgctagattcagcggcagcggcagcggcaccgagttcaccctgaccatcagcagcctgcagagcgaggacttcgccgtgtactattgccaacaagactacacaagcccctggaccttcggccaaggaaccaagctggagatcaagggcggctgccccccctgcccaccctgctga

SEQ ID NO: 61 - DNA, 789 bp, synthetic construct
  source 1..789: mol_type = other DNA
atggagaccgacaccctgctgctgtgggtgctgctcctgtgggtgcccggtagcaccggcgagatcgtgatgacacagagccccgcgaccctcagcgtcagtcccggcgagagagccacgctgagctgcaaggcatctcagagcgtgagcaacgacgtggtgtggtatcagcagaagcccggccaagcccctagactgctgatctacgacgctagcaacagatacaccggcatccccgctagattcagcggcagcggcagcggcaccgagttcaccctgaccatcagcagcctgcagagcgaggacttcgctgtgtactactgccaacaagactacacaagcccctggaccttcgggcaaggcaccaaactggaaattaagggcggaggtagcggaggtggtgggcaagtgcagctggtgcagagcggcgccgaggtgaagaagcccggcgctagcgtgaaagtgagctgcaaggcctccggatacaccttcaccaactacggcatgaactgggtgagacaagcccccggccaaggcctggagtggatgggctggatcaacacctacaccggcgagcccacctacgccgacgacttcaagggcagagtgaccatgaccaccgacacaagcacaagcaccgcctacatggagctgagaagcctgagaagcgacgacaccgccgtgtactattgcgcgcggatcggcgacagcagccctagcgactactggggccaaggcaccctggttaccgtgagcagcggcggctgccccccctgcccaccctgctga

SEQ ID NO: 62 - DNA, 789 bp, synthetic construct
  source 1..789: mol_type = other DNA
atggagaccgacaccctgctgctgtgggtgctcctgctgtgggttccgggatccaccggccaagtgcagctggtgcagagcggcgccgaggtgaagaagcccggcgctagcgtgaaggtaagctgcaaggctagcgggtacaccttcaccaactacggcatgaactgggtgagacaagcccccggccaaggcctggagtggatgggctggatcaacacctacaccggcgagcccacctacgccgacgacttcaagggcagagtgaccatgaccaccgacacaagcacaagcaccgcctacatggagctgagaagcctgagaagcgacgacaccgctgtgtactactgcgctcgcatcggcgacagcagccctagcgactactgggggcaaggcacccttgtcaccgtgagctcggggggcggaagcggtggcggcggtgagatcgtgatgacacagagccccgctacactgagcgtgagccccggcgagagagccacgctgagctgcaaagcctctcagagcgtgagcaacgacgtggtgtggtatcagcagaagcccggccaagcccctagactgctgatctacgacgctagcaacagatacaccggcatccccgctagattcagcggcagcggcagcggcaccgagttcaccctgaccatcagcagcctgcagagcgaggacttcgccgtgtactattgccaacaagactacacaagcccctggaccttcggccaaggaaccaagctggagatcaagggcggctgccccccctgcccaccctgctga

SEQ ID NO: 63 - DNA, 1191 bp, synthetic construct
  source 1..1191: mol_type = other DNA
atggagaccgacaccctgctgctgtgggtgctcctactgtgggtgcccggcagcaccggcgagatcgtgatgacacagagccccgcgacactcagcgtgagtcccggcgagagagcgacgctcagctgcaaggcaagccaaagcgtgagcaacgacgtggtgtggtatcagcagaagcccggccaagcccctcaactgctgatctacgacgctagcaacagatacaccggcatccccgctagattcagcggcagcggcagcggcaccgagttcaccctgaccatcagcagcctgcagagcgaggacttcgccgtttactactgtcaacaagactacacaagcccctggaccttcggccaaggcaccaagctggaaattaagggcagcacctctggaggcgggtcgggcggaggatcgggaggcggcggaagcagccaagtgcagctggtgcagagcggcgccgaggtgaagaagcccggcgctagcgtgaaggtatcttgcaaggctagcgggtataccttcaccaactacggcatgaactgggtgagacaagcccccggccaaggcctggagtggatgggctggatcaacacctacaccggcgagcccacctacgccgacgacttcaagggcagagtgaccatgaccaccgacacaagcacaagcaccgcctacatggagctgagaagcctgagaagcgacgacaccgctgtgtattattgcgcgcggatcggcgacagcagccctagcgactactggggacaaggcaccctggttaccgtgagcagcgaacccaagagctccgacaagacccacacgtgtcccccttgccctccttgtggcggaggaagctccggaggcggaagcggaggacagcctagggagccccaggtgtataccctccccccctccagggaagagatgaccaagaaccaggtgagcctgacctgcctcgtgaagggcttttatccctccgatatcgccgtggagtgggagagcaacggccagcctgagaacaattacaagaccaccccccctgtgctggactccgatggcagcttcttcctgtattccaagctgaccgtcgacaagtccaggtggcaacagggcaacgtcttcagctgcagcgtgatgcacgaggccctgcacaatcactacacccagaagtccctctccctgagccccggctga

SEQ ID NO: 64 - DNA, 1191 bp, synthetic construct
  source 1..1191: mol_type = other DNA
atggagaccgacaccctgctgctgtgggtgctcctactgtgggtgcccggcagcaccggcgagatcgtgatgacacagagccccgcgacactcagcgtgagtcccggcgagagagcgacgctcagctgcaaggcaagccaaagcgtgagcaacgacgtggtgtggtatcagcagaagcccggccaaagccctcaactgctgatctacgacgctagcaacagatacaccggcatccccgctagattcagcggcagcggcagcggcaccgagttcaccctgaccatcagcagcctgcagagcgaggacttcgccgtttactactgtcaacaagactacacaagcccctggaccttcggccaaggcaccaagctggaaattaagggcagcacctctggaggcgggtcgggcggaggatcgggaggcggcggaagcagccaagtgcagctggtgcagagcggcgccgaggtgaagaagcccggcgctagcgtgaaggtatcttgcaaggctagcgggtataccttcaccaactacggcatgaactgggtgagacaagcccccggccaaggcctggagtggatgggctggatcaacacctacaccggcgagcccacctacgccgacgacttcaagggcagagtgaccatgaccaccgacacaagcacaagcaccgcctacatggagctgagaagcctgagaagcgacgacaccgctgtgtattattgcgcgcggatcggcgacagcagccctagcgactactggggacaaggcaccctggttaccgtgagcagcgaacccaagagctccgacaagacccacacgtgtcccccttgccctccttgtggcggaggaagctccggaggcggaagcggaggacagcctagggagccccaggtgtataccctccccccctccagggaagagatgaccaagaaccaggtgagcctgacctgcctcgtgaagggcttttatccctccgatatcgccgtggagtgggagagcaacggccagcctgagaacaattacaagaccaccccccctgtgctggactccgatggcagcttcttcctgtattccaagctgaccgtcgacaagtccaggtggcaacagggcaacgtcttcagctgcagcgtgatgcacgaggccctgcacaatcactacacccagaagtccctctccctgagccccggctga

SEQ ID NO: 65 - protein, 107 aa, synthetic construct
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 107 aa, synthetic construct
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 107 aa, synthetic construct
GQPREPQVYTLPPSRDELTKNQVSLTCLVKGFYPSDIAVEWESNGQPENNYKTTPPVLDSDGSFFLYSKLTVDKSRWQQGNIFSCSVMHEALHNHYTQKSLSLSPGK

SEQ ID NO: 68 - protein, 107 aa, synthetic construct
GQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 69 - protein, 107 aa, synthetic construct
GQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 70 - protein, 107 aa, synthetic construct
GQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 71 - protein, 107 aa, synthetic construct
GQPREPQVYTLPPSREEMTKNQVSLTCLVKGFYPSDIAVEWESNGQPENNYNTTPPMLDSDGSFFLYSKLTVDKSRWQQGNVFSCSVMHEALHNHYTQKSLSLSPGK

SEQ ID NO: 72 - protein, 107 aa, synthetic construct
GQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 73 - protein, 107 aa, synthetic construct
GQPREPQVYTLPPSREEMTKNQVSLTCLVKGFYPSDIAVEWESSGQPENNYNTTPPVLDSDGSFFLYSRLTVDKSRWQEGNVFSCSVMHEALHNRFTQKSLSLSPGK

SEQ ID NO: 74 - protein, 107 aa, synthetic construct
GQPREPQVYTLPPSREEMTKNQVSLTCLVKGFYPSDIAVEWESSGQPENNYKTTPPMLDSDGSFFLYSKLTVDKSRWQQGNIFSCSVMHEALHNRFTQKSLSLSPGK

SEQ ID NO: 75 - protein, 107 aa, synthetic construct
GQPREPQVYTLPPSREEMTKNQVSLTCLVKGFYPSDIAVEWESNGQPENNYNTTPPMLDSDGSFFLYSKLTVDKSRWQQGNIFSCSVMHEALHNRFTQKSLSLSPGK

SEQ ID NO: 76 - protein, 107 aa, synthetic construct
GQPREPQVYTLPPSREEMTKNQVSLTCLVKGFYPSDIAVEWESSGQPENNYKTTPPMLDSDGSFFLYSKLTVDKSRWQEGNIFSCSVMHEALHNRFTQKSLSLSPGK

SEQ ID NO: 77 - protein, 107 aa, synthetic construct
GQPREPQVYTLPPSREEMTKNQVSLTCLVKGFYPSDIAVEWESNGQPENNYNTTPPMLDSDGSFFLYSKLTVDKSRWQQGNIFSCSVMHEALHNRYTQKSLSLSPGK

SEQ ID NO: 78 - protein, 107 aa, synthetic construct
GQPREPQVYTLPPSREEMTKNQVSLTCLVKGFYPSDIAVEWESNGQPENNYKTTPPMLDSDGSFFLYSKLTVDKSRWQQGNIFSCSVMHEALHNRYTQKSLSLSPGK

SEQ ID NO: 79 - protein, 107 aa, synthetic construct
GQPREPQVYTLPPSREEMTKNQVSLTCLVKGFYPSDIAMEWESSGQPENNYKTTPPVLDSDGSFFLYSKLTVDKSRWQQGNIFSCSVMHEALHNHYTQKSLSLSPGK

SEQ ID NO: 80 - protein, 107 aa, synthetic construct
GQPREPQVYTLPPSREEMTKNQVSLTCLVKGFYPSDIAVEWESNGQRENNYNTTPPMLDSDGSFFLYSKLTVDKSRWQQGNIFSCSVMHEALHNRYTQKSLSLSPGK

SEQ ID NO: 81 - protein, 107 aa, synthetic construct
GQPREPQVYTLPPSREEMTKNQVSLTCLVKGFYPSDIAVEWESNGQPENNYNTTPPMLDSDGSFFLYSKLTVDKSRWQQGNIFSCSVMHEALHNHYTQKSLSLSPGK

SEQ ID NO: 82 - protein, 107 aa, synthetic construct
GQPREPQVYTLPPSREEMTKNQVSLTCLVKGFYPSDIAVEWESSGQPENNYKTTPPVLDSDGSFFLYSKLTVDKSRWQQGNIFSCSVMHEALHNHYTQKSLSLSPGK

SEQ ID NO: 83 - protein, 107 aa, synthetic construct
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 84 - protein, 107 aa, synthetic construct
GQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 85 - protein, 107 aa, synthetic construct
GQPREPQVYTLPPSREEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 86 - protein, 118 aa, synthetic construct
  source 1..118: note = Rovalpituzimab-HC
QVQLVQSGAEVKKPGASVKVSCKASGYTFTNYGMNWVRQAPGQGLEWMGWINTYTGEPTYADDFKGRVTMTTDTSTSTAYMELRSLRSDDTAVYYCARIGDSSPSDYWGQGTLVTVSS

SEQ ID NO: 87 - protein, 118 aa, synthetic construct; identical to SEQ ID NO: 86
  source 1..118: note = IAB57M1-3-HC

SEQ ID NO: 88 - protein, 118 aa, synthetic construct; identical to SEQ ID NO: 86
  source 1..118: note = IAB57M2-6-HC

SEQ ID NO: 89 - protein, 107 aa, synthetic construct; identical to SEQ ID NO: 49
  source 1..107: note = Rovalpitizumab-LC

SEQ ID NO: 90 - protein, 106 aa, synthetic construct
  source 1..106: note = IAB57M1-3-LC
IVMTQSPATLSVSPGERATLSCKASQSVSNDVVWYQQKPGQAPRLLIYDASNRYTGIPARFSGSGSGTEFTLTISSLQSEDFAVYYCQQDYTSPWTFGQGTKLEIK

SEQ ID NO: 91 - protein, 106 aa, synthetic construct
  source 1..106: note = IAB57M2-6-LC
IVMTQSPATLSVSPGERATLSCKASQSVSNDVVWYQQKPGQAPRLLIYYASNRNTGIPARFSGSGSGTEFTLTISSLQSEDFAVYYCQQDYTSPWTFGQGTKLEIK

SEQ ID NO: 92 - skipped

SEQ ID NO: 93 - protein, 9 aa, synthetic construct
GGCPPCPPC

SEQ ID NO: 94 - protein, 7 aa, synthetic construct
KPGKAPK

SEQ ID NO: 95 - protein, 7 aa, synthetic construct
KPGQAPR

SEQ ID NO: 96 - protein, 7 aa, synthetic construct
KPEKAPK

SEQ ID NO: 97 - protein, 7 aa, synthetic construct
KPGKVPK

SEQ ID NO: 98 - protein, 7 aa, synthetic construct
KPGQPPR

SEQ ID NO: 99 - protein, 7 aa, synthetic construct
KPGQSPR

SEQ ID NO: 100 - protein, 7 aa, synthetic construct
KPGLAPR

SEQ ID NO: 101 - protein, 7 aa, synthetic construct
KPGQPPK

SEQ ID NO: 102 - protein, 10 aa, synthetic construct
EPKSSDKTHT

SEQ ID NO: 103 - protein, 10 aa, synthetic construct
EPGSSDGTHT

SEQ ID NO: 104 - protein, 7 aa, synthetic construct
CPPCPPC

SEQ ID NO: 105 - protein, 5 aa, synthetic construct
CPPCP

SEQ ID NO: 106 - protein, 8 aa, synthetic construct
APELLGGP

SEQ ID NO: 107 - protein, 10 aa, synthetic construct
GGGSSGGGSG

SEQ ID NO: 108 - protein, 7 aa, synthetic construct
APPVAGP

SEQ ID NO: 109 - protein, 225 aa, synthetic construct
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 110 - protein, 7 aa, synthetic construct
KPGQSPQ

SEQ ID NO: 111 - protein, 7 aa, synthetic construct
KPGQAPQ

SEQ ID NO: 112 - protein, 10 aa, synthetic construct
EPKSCDKTHT

SEQ ID NO: 113 - skipped

SEQ ID NO: 114 - protein, 12 aa, synthetic construct
ELKTPLGDTTHT

SEQ ID NO: 115 - protein, 7 aa, synthetic construct
ESKYGPP

SEQ ID NO: 116 - protein, 4 aa, synthetic construct
CPPC

SEQ ID NO: 117 - protein, 10 aa, synthetic construct
CPPCVECPPC

SEQ ID NO: 118 - protein, 10 aa, synthetic construct
CPPCPPCPPC

SEQ ID NO: 119 - protein, 9 aa, synthetic construct
CCVECPPCP

SEQ ID NO: 120 - protein, 9 aa, synthetic construct
SCVECPPCP

SEQ ID NO: 121 - protein, 7 aa, synthetic construct
CVECPPC

SEQ ID NO: 122 - protein, 10 aa, synthetic construct
CPPCPPCPPC

SEQ ID NO: 123 - protein, 23 aa, synthetic construct
  source 1..23: note = Human IgG1 NH1
EPKSCDKTHTCPPCPAPELLGGP

SEQ ID NO: 124 - protein, 24 aa, synthetic construct
  source 1..24: note = Human IgG1 EH1
EPKSCDKTHTCPPCGGGSSGGGSG

SEQ ID NO: 125 - protein, 23 aa, synthetic construct
  source 1..23: note = Human IgG1 NH2
EPKSSDKTHTCPPCPAPELLGGP

SEQ ID NO: 126 - protein, 24 aa, synthetic construct
  source 1..24: note = Human IgG1 EH2
EPKSSDKTHTCPPCGGGSSGGGSG

SEQ ID NO: 127 - protein, 25 aa, synthetic construct
  source 1..25: note = Human IgG1 NH3
EPKSSDKTHTCPPCPPCAPELLGGP

SEQ ID NO: 128 - protein, 27 aa, synthetic construct
  source 1..27: note = Human IgG1 EH3
EPKSSDKTHTCPPCPPCGGGSSGGGSG

SEQ ID NO: 129 - protein, 28 aa, synthetic construct
  source 1..28: note = Human IgG1 NH4
EPKSSDKTHTCPPCVECPPCAPELLGGP

SEQ ID NO: 130 - protein, 30 aa, synthetic construct
  source 1..30: note = Human IgG1 EH4
EPKSSDKTHTCPPCVECPPCGGGSSGGGSG

SEQ ID NO: 131 - protein, 28 aa, synthetic construct
  source 1..28: note = Human IgG1 NH5
EPKSSDKTHTCPPCPPCPPCAPELLGGP

SEQ ID NO: 132 - protein, 30 aa, synthetic construct
  source 1..30: note = Human IgG1 EH5
EPKSSDKTHTCPPCPPCPPCGGGSSGGGSG

SEQ ID NO: 133 - protein, 19 aa, synthetic construct
  source 1..19: note = Human IgG2 NH1
ERKCCVECPPCPAPPVAGP

SEQ ID NO: 134 - protein, 22 aa, synthetic construct
  source 1..22: note = Human IgG2 EH1
ERKCCVECPPCPGGGSSGGGSG

SEQ ID NO: 135 - protein, 19 aa, synthetic construct
  source 1..19: note = Human IgG2 NH2
ERKSCVECPPCPAPPVAGP

SEQ ID NO: 136 - protein, 22 aa, synthetic construct
  source 1..22: note = Human IgG2 EH2
ERKSCVECPPCPGGGSSGGGSG

SEQ ID NO: 137 - protein, 29 aa, synthetic construct
  source 1..29: note = IgG3/IgG1 EH6
ELKTPLGDTTHTCVECPPCGGGSSGGGSG

SEQ ID NO: 138 - protein, 29 aa, synthetic construct
  source 1..29: note = IgG3/IgG1 EH7
ELKTPLGDTTHTCPPCPPCGGGSSGGGSG

SEQ ID NO: 139 - protein, 32 aa, synthetic construct
  source 1..32: note = IgG3/IgG1 EH8
ELKTPLGDTTHTCPPCPPCPPCGGGSSGGGSG

SEQ ID NO: 140 - protein, 20 aa, synthetic construct
  source 1..20: note = IgG4 NH
ESKYGPPCPPCPAPELLGGP

SEQ ID NO: 141 - protein, 22 aa, synthetic construct
  source 1..22: note = IgG4 EH
ESKYGPPCPPCPGGGSSGGGSG

SEQ ID NO: 142 - protein, 376 aa, synthetic construct
EIVMTQSPATLSVSPGERATLSCKASQSVSNDVVWYQQKPGQAPRLLIYDASNRNTGIPARFSGSGSGTEFTLTISSLQSEDFAVYYCQQDYTSPWTFGQGTKLEIKGSTSGGGSGGGSGGGGSSQVQLVQSGAEVKKPGASVKVSCKASGYTFTNYGMNWVRQAPGQGLEWMGWINTYTGEPTYADDFKGRVTMTTDTSTSTAYMELRSLRSDDTAVYYCARIGDSSPSDYWGQGTLVTVSSEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 143-145 - skipped (3 entries)

SEQ ID NO: 146 - protein, 7 aa, synthetic construct
KPGQAPK

SEQ ID NO: 147 - protein, 9 aa, synthetic construct
QQKPGQSPQ

(19) United States
(12) Patent Application Publication
(10) Pub. No.: US 2026/0234226 A1
(43) Pub. Date: Aug. 13, 2026

(54) DLL3 ANTIGEN BINDING CONSTRUCTS

(71) Applicant: ImaginAb, Inc., Inglewood, CA (US)

(72) Inventors: Alessandro Mascioni, Los Angeles, CA (US); Fang Jia, Cerritos, CA (US); Leticia Maria De Souza Cordeiro, Redondo Beach, CA (US); Kelley C. Diaz, Anaheim, CA (US); Patrick Joyce, Gardena, CA (US); Argin Aivazian, Burbank, CA (US)

(21) Appl. No.: 18/881,540

(22) PCT Filed: Jul. 6, 2023
(86) PCT No.: PCT/US2023/069710

§ 371 Date: Jan. 6, 2025

Related U.S. Application Data
(60) Provisional application No. 63/367,953, filed on Jul. 8, 2022.

(51) Int. Cl. C07K 16/18; A61K 47/68; A61K 49/00; A61K 51/10

(52) U.S. Cl. CPC C07K 16/18; A61K 47/6801; A61K 49/0058; A61K 51/1018; C07K 2317/24; C07K 2317/622; C07K 2317/626

(57) ABSTRACT

Provided herein are components for antigen binding constructs, including antibodies and fragments thereof, such as minibodies and cys-diabodies, that bind to a target molecule, for example, DLL3. In some embodiments, these components are novel complementarity-determining region (CDR) sequences and/or sequences associated with and/or part of the CDR sequence. In some embodiments, these components are novel framework region (FR) sequences and/or sequences associated with and/or part of the FR sequence. These CDR and FR sequences can provide various benefits. Also provided herein are the antigen binding constructs (such as, minibodies, cys-diabodies, etc.) that include one or more of the CDR or FR sequences or subsequences provided herein.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### REFERENCE TO SEQUENCE LISTING

The present application is being filed along with a Sequence Listing in electronic format. The Sequence Listing is provided as a file entitled Seglist_IGNAB059WO.xml, which was created and last modified on Jun. 30, 2023, which is 155,593 bytes in size. The information in the electronic Sequence Listing is hereby incorporated by reference in its entirety.

### FIELD

Embodiments herein relate to antigen binding constructs, minibodies, and cys-diabodies. Specifically, DLL3 specific antigen binding constructs, minibodies, and cys-diabodies.

### BACKGROUND

Delta-like protein 3 (DLL3) is an inhibitory protein of the Notch signaling pathway. DLL3 is expressed on the cell surface of small cell lung cancer (SCLC) and other high-grade endocrine tumors.

### SUMMARY

Embodiments herein relate to antigen binding constructs, minibodies, and cys-diabodies. Some embodiments are DLL3 specific antigen binding constructs, minibodies, and cys-diabodies. Some embodiments herein relate to therapeutic agents, formulations, and compositions, comprising Dll3 specific antigen binding constructs, minibodies, and cys-diabodies. Some embodiments herein are related to methods of treating a DLL3 associated disease or condition using the antigen binding constructs, minibodies, and cys-diabodies, disclosed herein.

Provided herein is an antigen binding construct that comprises: a variable light (V_{L}) domain comprising: a LCDR1 that is SEQ ID NO: 15; a LCDR2 that is any one of SEQ ID NO: 19, 21, or 23; a LCDR3 that is SEQ ID NO: 25; and/or a variable heavy (V_{H}) domain comprising: a HCDR1 that is SEQ ID NO: 27; a HCDR2 that is SEQ ID NO: 29; and a HCDR3 that is SEQ ID NO: 31.

Provided herein is a minibody that binds to DLL3, the minibody comprising: a single-chain variable fragment (scFv) that binds to DLL3, the scFv comprising a variable light (V_{L}) domain linked to a variable heavy (V_{H}) domain, the V_{L} domain comprising: a LCDR1 that is SEQ ID NO: 15; a LCDR2 that is any one of SEQ ID NO: 19, 21, or 23; a LCDR3 that is SEQ ID NO: 25; and the V_{H} domain comprising: a HCDR1 that is SEQ ID NO: 27; a HCDR2 that is SEQ ID NO: 29; a HCDR3 that is SEQ ID NO: 31; a hinge-extension domain comprising a hinge region; and a IgG C_{H}3 sequence.

Provided herein is a cys-diabody that binds to DLL3, the cys-diabody comprising a polypeptide that comprises: a single-chain variable fragment (scFv) comprising a variable light (V_{L}) domain linked to a variable heavy (V_{H}) domain; and the V_{L} domain comprising: a LCDR1 that is SEQ ID NO: 15; a LCDR2 that is any one of SEQ ID NO: 19, 21, or 23; a LCDR3 that is SEQ ID NO: 25; and the V_{H} domain comprising: a HCDR1 that is SEQ ID NO: 27; a HCDR2 that is SEQ ID NO: 29; and a HCDR3 that is SEQ ID NO: 31.

The antigen binding construct, minibody, or cys-diabody of any one of the preceding embodiments, wherein the V_{L} domain further comprises a LFR2 of SEQ ID NO: 35 or 37.

The antigen binding construct, minibody, or cys-diabody of any one of the preceding embodiments; wherein the V_{L} domain comprises an amino acid sequence having at least 90% identity to a V_{L} domain in SEQ ID NO: 48; and wherein the antigen binding construct, minibody, or cys-diabody comprises a Y50D and/or Y55N mutation (Kabat).

The antigen binding construct, minibody, or cys-diabody of any one of the preceding embodiments; wherein the V_{L} domain comprises an amino acid sequence having at least 90% identity to a V_{L} domain in SEQ ID NO: 48; and wherein the antigen binding construct, minibody, or cys-diabody comprises a A43S and/or Q45R mutation (Kabat).

The antigen binding construct, minibody, or cys-diabody of any one of the preceding embodiments; wherein the V_{L} domain comprises an amino acid sequence having at least 90% identity to a V_{L} domain in SEQ ID NO: 49 and wherein the antigen binding construct, minibody, or cys-diabody comprises a Y50D and/or Y55N mutation (Kabat).

The antigen binding construct, minibody, or cys-diabody of any one of the preceding embodiments; wherein the V_{L} domain comprises an amino acid sequence having at least 90% identity to a V_{L} domain in SEQ ID NO: 49; and wherein the antigen binding construct, minibody, or cys-diabody comprises a A43S and/or Q45R mutation (Kabat).

The antigen binding construct, minibody, or cys-diabody of any one of the preceding embodiments; wherein the antigen binding construct, minibody, or cys-diabody, further comprises a signal peptide that is SEQ ID NO: 45.

The antigen binding construct, minibody, or cys-diabody of any one of the preceding embodiments; wherein the antigen binding construct, minibody, or cys-diabody, comprise a linker that is any one of SEQ ID NO: 39-44.

The antigen binding construct or minibody of any one of the preceding embodiments; wherein the C_{H}3 domain is an IgG C_{H}3 domain.

The antigen binding construct or minibody of any one of the preceding embodiments; wherein the IgG C_{H}3 domain is an IgG1, IgG2, IgG3, or IgG4 C_{H}3 domain.

The antigen binding construct or minibody of any one of the preceding embodiments; wherein the IgG C_{H}3 domain comprises an IgG1 C_{H}3 domain that is any one of SEQ ID NOs: 65-68.

The antigen binding construct or minibody of any one of the preceding embodiments; wherein the IgG C_{H}3 domain comprises an IgG1 C_{H}3 domain that is any one of SEQ ID NOs: 69-71.

The antigen binding construct or minibody of any one of the preceding embodiments; wherein the IgG C_{H}3 domain comprises an IgG1 C_{H}3 domain that is any one of SEQ ID NOs: 72-82.

The antigen binding construct or minibody of any one of the preceding embodiments; wherein the IgG C_{H}3 domain comprises an IgG1 C_{H}3 domain that is any one of SEQ ID NO: 83-85.

The antigen binding construct of any one of the preceding embodiments, wherein the antigen binding construct is an antibody.

The antigen binding construct, minibody, or cys-diabody of any one of the preceding embodiments, wherein the antigen binding construct, minibody, or cys-diabody, binds specifically to DLL3.

The antigen binding construct, minibody, or cys-diabody of any one of the preceding embodiments, further comprising a detectable marker.

The antigen binding construct, minibody, or cys-diabody of any one of the preceding embodiments, wherein the detectable marker is a fluorescently detectable marker.

The antigen binding construct, minibody, or cys-diabody of any one of the preceding embodiments, wherein the detectable marker comprises a phototherapy compatible dye.

The antigen binding construct, minibody, or cys-diabody of any one of the preceding embodiments, wherein the detectable marker is compatible for use with Boron Neutron Capture therapy (BNCT).

The antigen binding construct, minibody, or cys-diabody of any one of the preceding embodiments, wherein the detectable marker is a radiolabel.

The antigen binding construct, minibody, or cys-diabody of any one of the preceding embodiments, wherein the detectable marker is an alpha-emitter radiolabel.

The antigen binding construct, minibody, or cys-diabody of any one of the preceding embodiments, wherein the detectable marker is a beta-emitter radiolabel.

The antigen binding construct, minibody, or cys-diabody of any one of the preceding embodiments, wherein the detectable marker is an positron-emitter radiolabel.

The antigen binding construct, minibody, or cys-diabody of any one of the preceding embodiments, wherein the detectable marker is a gamma-emitter radiolabel.

The antigen binding construct, minibody, or cys-diabody of any one of the preceding embodiments, wherein the detectable marker comprises Lutetium-177.

The antigen binding construct, minibody, or cys-diabody of any one of the preceding embodiments, wherein the detectable marker is suitable for use with Auger electron spectroscopy.

The antigen binding construct, minibody, or cys-diabody of any one of the preceding embodiments, wherein the detectable marker comprises an isotope.

The antigen binding construct, minibody, or cys-diabody of any one of the preceding embodiments, wherein the detectable marker comprises a bioluminescent compound.

The antigen binding construct, minibody, or cys-diabody of any one of the preceding embodiments, wherein the detectable marker comprises a chemiluminescent compound.

The antigen binding construct, minibody, or cys-diabody of any one of the preceding embodiments, wherein the detectable marker comprises an enzyme.

The antigen binding construct, minibody, or cys-diabody of any one of the preceding embodiments, wherein the detectable marker comprises a metal chelator.

The antigen binding construct, minibody, or cys-diabody of any one of the preceding embodiments, further comprising a therapeutic agent.

The antigen binding construct, minibody, or cys-diabody of any one of the preceding embodiments, wherein the therapeutic agent comprises a therapeutic isotope or ion.

The antigen binding construct, minibody, or cys-diabody of any one of the preceding embodiments, wherein the therapeutic agent is a radiolabel.

The antigen binding construct, minibody, or cys-diabody of any one of the preceding embodiments, wherein the therapeutic agent is an alpha-emitter radiolabel.

The antigen binding construct, minibody, or cys-diabody of any one of the preceding embodiments, wherein the therapeutic agent is a beta-emitter radiolabel.

The antigen binding construct, minibody, or cys-diabody of any one of the preceding embodiments, wherein the therapeutic agent is an positron-emitter radiolabel.

The antigen binding construct, minibody, or cys-diabody of any one of the preceding embodiments, wherein the therapeutic agent is a gamma-emitter radiolabel.

The antigen binding construct, minibody, or cys-diabody of any one of the preceding embodiments, wherein the therapeutic agent comprises Lutetium-177.

The antigen binding construct, minibody, or cys-diabody of any one of the preceding embodiments, wherein the therapeutic agent comprises a phototherapy compatible dye.

The antigen binding construct, minibody, or cys-diabody of any one of the preceding embodiments, wherein the therapeutic agent comprises boron.

The antigen binding construct, minibody, or cys-diabody of any one of the preceding embodiments, wherein the therapeutic agent is compatible for use with Boron Neutron Capture therapy (BNCT).

The antigen binding construct, minibody, or cys-diabody of any one of the preceding embodiments, wherein the detectable marker and/or therapeutic agent comprises a toxic payload.

The antigen binding construct, minibody, or cys-diabody of any one of the preceding embodiments, wherein the antigen binding construct, minibody, or cys-diabody, is bispecific.

The antigen binding construct, minibody, or cys-diabody of any one of the preceding embodiments, wherein the antigen binding construct, minibody, or cys-diabody comprises a monovalent scFv.

The antigen binding construct, minibody, or cys-diabody of any one of the preceding embodiments, from N terminus to C terminus of the polypeptide is V_{L}, V_{H}.

The antigen binding construct, minibody, or cys-diabody of any one of the preceding embodiments, wherein the order of the variable domains, from N terminus to C terminus of the polypeptide is V_{H}, V_{L}.

The antigen binding construct, minibody, or cys-diabody of any one of the preceding embodiments, wherein the minibody is a humanized antigen binding construct, minibody, or cys-diabody.

The antigen binding construct, minibody, or cys-diabody of any one of the preceding embodiments, wherein the humanized antigen binding construct, minibody, or cys-diabody comprises: a LCDR1 that is SEQ ID NO: 15; a LCDR2 that is any one of SEQ ID NO: 19, 21, or 23; a LCDR3 that is SEQ ID NO: 25; a HCDR1 that is SEQ ID NO: 27; a HCDR2 that is SEQ ID NO: 29; and a HCDR3 that is SEQ ID NO: 31.

The antigen binding construct, minibody, or cys-diabody any one of the preceding embodiments, wherein the V_{L} domain further comprises a LFR2 of a LFR2 of SEQ ID NO: 35 or 37.

The antigen binding construct, minibody, or cys-diabody of any one of the preceding embodiments, wherein the antigen binding construct, minibody, or cys-diabody, has increased expression yield in mammalian cells as compared to an antigen binding construct, minibody, or cys-diabody comprising SEQ ID NO: 1.

The antigen binding construct, minibody, or cys-diabody of any one of the preceding embodiments, wherein the antigen binding construct, minibody, or cys-diabody, has between about 75% and 300%, 75% 200%, 75% and 150%, or 75% and 100% expression yield as compared to an antigen binding construct, minibody, or cys-diabody comprising SEQ ID NO: 1.

The antigen binding construct, minibody, or cys-diabody of any one of the preceding embodiments, wherein the antigen binding construct, minibody, or cys-diabody, has about 75%, 80%, 85%, 90%, 95%, 100%, 105%, 110%, 115%, 120%, 125%, 150%, 175%, 200%, 250%, or 300% expression yield as compared to an antigen binding construct, minibody, or cys-diabody comprising SEQ ID NO: 1, or a percentage in a range that is defined by any two of the preceding values (e.g., about 75-300%, about 80-250%, about 100%-300%, about 110%-250%, about 120%-200%, etc.).

The antigen binding construct, minibody, or cys-diabody of any one of the preceding embodiments, wherein the antigen binding construct, minibody, or cys-diabody, accumulates to a detectable level in a subject's blood, liver, kidney, spleen, lungs, muscle, bone, heart, stomach, small intestine, large intestine, pancreas, tumor, or any combination therein.

The antigen binding construct, minibody, or cys-diabody of any one of the preceding embodiments, wherein the subject is a mammal.

The antigen binding construct, minibody, or cys-diabody of any one of the preceding embodiments, wherein the antigen binding construct, minibody, or cys-diabody is detectable at, at about, or at up to about 0.1%, 0.5%, 1%, 2%, 3%. 4%, 5%, 6%, 7%, 8%, 9%, 10%, 15%, 20%, 25%, 30%, or 35% ID/g, or a percentage in a range that is defined by any two of the preceding values.

The antigen binding construct, minibody, or cys-diabody of any one of the preceding embodiments, wherein the antigen binding construct, minibody, or cys-diabody is detectable at between about 0.1% and 35%, 0.1% and 30%, 1% and 35%, 1% and 30%, 5% and 35%, or 5% and 30% ID/g.

The antigen binding construct, minibody, or cys-diabody of any one of the preceding embodiments, wherein the antigen binding construct, minibody, or cys-diabody, comprises a LFR2 of a LFR2 of SEQ ID NO: 35 or 37; and wherein the antigen binding construct, minibody, or cys-diabody has improved biodistribution as compared to an antigen binding construct, minibody, or cys-diabody comprising an LFR2 that is a LFR2 of SEQ ID NO: 33.

The antigen binding construct, minibody, or cys-diabody of any one of the preceding embodiments, wherein the antigen binding construct, minibody, or cys-diabody, has a KD of less than about 1×10⁻¹⁰ M.

The antigen binding construct, minibody, or cys-diabody of any one of the preceding embodiments, wherein the antigen binding construct, minibody, or cys-diabody, has a KD of less than about 1×10⁻¹² M.

The antigen binding construct, minibody, or cys-diabody of any one of the preceding embodiments, wherein the antigen binding construct, minibody, or cys-diabody, has an EC₅₀ of, of about, or of at most 0.001, 0.005, 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, or 0.20 nM, or an EC₅₀ in a range that is defined by any two of the preceding values.

The antigen binding construct, minibody, or cys-diabody of any one of the preceding embodiments, wherein the antigen binding construct, minibody, or cys-diabody, has an EC₅₀ of between about 0.001 and 0.20 nM, 0.001 and 0.17 nM, 0.001 and 0.12 nM, 0.01 and 0.20 nM, 0.01 and 0.17 nM, 0.01 and 0.12 nM, 0.05 and 0.20 nM, 0.05 and 0.17 nM. 0.05 and 0.15 nM, 0.05 and 0.12 nM, 0.08 and 0.20 nM, 0.08 and 0.17 nM, or 0.08 and 0.12 nM.

The antigen binding construct, minibody, or cys-diabody of any one of the preceding embodiments, wherein the antigen binding construct, minibody, or cys-diabody, has an EC₅₀ of up to about 10 nM.

Provided herein is a nucleic acid encoding an antigen binding construct, minibody, or cys-diabody, of any of the preceding embodiments.

The nucleic acid of any of the preceding embodiments, wherein the nucleic acid is any one of SEQ ID NO: 51-65.

Provided herein is a cell line producing an antigen binding construct, minibody, or cys-diabody, of any of the preceding embodiments.

Provided herein is a kit comprising: an antigen binding construct, minibody, or cys-diabody, of any of the preceding embodiments; and a chelator, wherein the chelator allows incorporation of a detectable marker.

Provided herein is a kit comprising: an antigen binding construct, minibody, or cys-diabody, of any of the preceding embodiments; and a chelator, wherein the chelator allows incorporation of a therapeutic isotope or agent.

Provided herein is a kit comprising: an antigen binding construct, minibody, or cys-diabody, of any of the preceding embodiments; and a linker, wherein the linker allows incorporation of a detectable marker.

Provided herein is a kit comprising: an antigen binding construct, minibody, or cys-diabody, of any of the preceding embodiments; and a linker, wherein the linker allows incorporation of a therapeutic isotope or agent.

Provided herein is a kit comprising: an antigen binding construct, minibody, or cys-diabody, of any of the preceding embodiments; and a detectable marker.

Provided herein is a method of detecting a presence or absence of a DLL3, the method comprising: applying the antigen binding construct, minibody, or cys-diabody, of any of the preceding embodiments to a sample; and detecting a presence or an absence of the antigen binding construct, thereby detecting a presence or absence of a DLL3.

The method of any one of the preceding embodiments, wherein the antigen binding construct, minibody, or cys-diabody, is conjugated to a detectable marker.

The method of any one of the preceding embodiments, wherein applying the antigen binding construct, minibody, or cys-diabody, comprises administering the antigen binding construct to a subject.

The method of any one of the preceding embodiments, wherein detecting binding or absence of binding of the antigen binding construct, minibody, or cys-diabody, to DLL3 comprises at least one of positron emission tomography, single-photon emission computed tomography, or fluorescent tomography.

The method of any one of the preceding embodiments, the method further comprising applying a secondary antigen binding construct to the sample, wherein the secondary antigen binding construct binds specifically to the antigen binding construct.

The method of any one of the preceding embodiments, wherein the antigen binding construct, minibody, or cys-diabody, is incubated with the sample for no more than 20 hours.

The method of any one of the preceding embodiments, wherein the antigen binding construct, minibody, or cys-diabody, is incubated with the sample for no more than 6 hours.

The method of any one of the preceding embodiments, wherein the antigen binding construct, minibody, or cys-diabody, is administered to a host, and wherein a first quantity of antigen binding construct, minibody, or cys-diabody, thereof is unbound to DLL3, and a second quantity of antigen binding construct, minibody, or cys-diabody, is bound to DLL3, wherein at least about 80% of the first quantity of antigen binding construct, minibody, or cys-diabody, is eliminated in no more than 12 hours.

Provided herein is a method of targeting a therapeutic agent to DLL3, the method comprising administering to a subject an antigen binding construct, minibody, or cys-diabody, of any one of the preceding claims, wherein the antigen binding construct is conjugated to a therapeutic agent.

Provided herein is a therapeutic composition targeting DLL3, wherein the therapeutic composition comprises: an antigen binding construct that comprises: a variable light (V_{L}) domain comprising: a LCDR1 that is SEQ ID NO: 15; a LCDR2 that is any one of SEQ ID NO: 19, 21, or 23; a LCDR3 that is SEQ ID NO: 25; and a variable heavy (V_{H}) domain comprising: a HCDR1 of a HCDR1 that is SEQ ID NO: 27; a HCDR2 of HCDR2 that is SEQ ID NO: 29; a HCDR3 of HCDR3 that is SEQ ID NO: 31; and a therapeutic agent, toxic payload, and/or a detectable marker.

Provided herein is a therapeutic composition targeting DLL3, wherein the therapeutic composition comprises: a minibody that binds to DLL3, the minibody comprising: a single-chain variable fragment (scFv) that binds to DLL3, the scFv comprising a variable light (V_{L}) domain linked to a variable heavy (V_{H}) domain, the V_{L} domain comprising: a LCDR1 that is SEQ ID NO: 15; a LCDR2 that is any one of SEQ ID NO: 19, 21, or 23; a LCDR3 that is SEQ ID NO: 25 and the V_{H} domain comprising: a HCDR1 that is SEQ ID NO: 27; a HCDR2 that is SEQ ID NO: 29; a HCDR3 that is SEQ ID NO: 31; a hinge-extension domain comprising a IgG1 hinge region; a IgG C_{H}3 sequence; and a therapeutic agent, toxic payload, and/or a detectable marker.

Provided herein is a therapeutic composition targeting DLL3, wherein the therapeutic composition comprises: a cys-diabody that binds to DLL3, the cys-diabody comprising a polypeptide that comprises: a single-chain variable fragment (scFv) comprising a variable light (Vy) domain linked to a variable heavy (Vy) domain; the Vy domain comprising: a LCDR1 that is SEQ ID NO: 15; a LCDR2 that is SEQ ID NO: 19, 21, or 23; a LCDR3 that is SEQ ID NO: 25; the Vy domain comprising: a HCDR1 that is SEQ ID NO: 27; a HCDR2 that is SEQ ID NO: 29; a HCDR3 that is SEQ ID NO: 31 and; a therapeutic agent, toxic payload, and/or a detectable marker.

The therapeutic composition of any of the preceding embodiments, wherein the detectable marker is a radiolabel.

The therapeutic composition of any of the preceding embodiments, wherein the detectable marker is an alpha-emitter radiolabel.

The therapeutic composition of any of the preceding embodiments, wherein the detectable marker is a beta-emitter radiolabel.

The therapeutic composition of any of the preceding embodiments, wherein the detectable marker is a positron-emitter radiolabel.

The therapeutic composition of any of the preceding embodiments, wherein the detectable marker comprises Lutetium-177.

### BRIEF DESCRIPTION OF THE DRAWINGS

FIG. 1 shows some embodiments of a polypeptide sequence of antigen binding minibodies. (SEQ ID NO: 1-10).

FIG. 2 shows some embodiments of a polypeptide sequence of antigen binding cys-diabodies. (SEQ ID NO: 11-14).

FIG. 3 shows some embodiments of a polypeptide sequence of a LCDR (SEQ ID NO: 15-26), HCDR (SEQ ID NO: 27-32), LFR (SEQ ID NO: 33-38), linker (SEQ ID NO: 39-44), signal peptide (SEQ ID NO: 45-46), parental V_{H} (SEQ ID NO: 47), and parental V_{L} (SEQ ID NO: 48).

FIG. 4 shows some embodiments of an annotated polypeptide sequence of an antigen binding minibody.

FIG. 5 shows some embodiments of a variable region alignment of the polypeptide sequence of antigen binding minibodies and the Rovalpituzumab polypeptide sequence.

FIG. 6 shows embodiments of a polypeptide sequence of human-DLL3. (SEQ ID NO: 50).

FIG. 7 is a bar graph showing embodiments of minibody expression level in mammalian cells.

FIG. 8 is a collection of graphs and corresponding tables showing some embodiments of EC₅₀ determinations of antigen binding minibodies by ELISA.

FIG. 9 is a collection of graphs and corresponding tables showing some embodiments of equilibrium dissociation constant determinations of antigen binding minibodies.

FIG. 10 is a collection of graphs showing some embodiments of EC₅₀ determinations of antigen binding minibodies by flow cytometry.

FIG. 11 is a bar graph showing some embodiments of the in-vivo biodistribution of an antigen binding minibody in tumor bearing mice models.

FIG. 12 is a bar graph showing some embodiments of the in-vivo biodistribution of antigen binding minibodies and cys-diabodies.

FIG. 13 shows some embodiments of a nucleic acid sequence of an antigen binding construct, minibody, and cys-diabody. (SEQ ID NO: 51-64).

FIG. 14 shows some embodiments of a C_{H}3 polypeptide sequence. (SEQ ID NO: 65-85).

### DETAILED DESCRIPTION

Described herein are antigen binding constructs, including antibodies and fragments thereof, such as minibodies and cys-diabodies. In some embodiments, these bind to a target molecule, for example, DLL3. In some embodiments, these components are novel complementarity-determining region (CDR) sequences and/or sequences associated with and/or part of the CDR sequence. In some embodiments, these components are novel framework region (FR) sequences and/or sequences associated with and/or part of the FR sequence. These CDR and FR sequences can provide various benefits and improvements over the art. Also provided herein are the antigen binding constructs (such as, minibodies, cys-diabodies, etc.) that include one or more of the CDR or FR sequences or subsequences provided herein.

In some embodiments, the antigen binding constructs can be useful for targeting therapeutic agents to cells that express the target molecule. In some embodiments, methods are provided for detecting the presence or absence of a target molecule (or “target”) using antigen binding constructs (including antibodies, minibodies, and cys-diabodies). In some embodiments, methods are provided for using the antigen binding constructs for therapeutic purposes.

In some embodiments, antigen binding constructs such as minibodies and cys-diabodies can have superior pharmacokinetic properties for faster diagnostic imaging while maintaining the binding specificity and affinity of the parental antibody. Current technology utilizes imaging with the full length antibodies which often requires significantly longer times (~7-8 days post-injection) to produce high contrast images due to the slow serum clearance of the intact antibody. Some embodiments of the antigen binding constructs, minibodies, and cys-diabodies provided herein provide the opportunity for same-day or next-day imaging.

In some embodiments, the antigen binding constructs are for diagnostics. When labeled with an appropriate radionuclides (e.g., the positron emitter Iodine-124, Copper-64, Fluorine-18, Gallium-68 and/or Zirconium-89 for PET imaging) or fluorophore (for fluorescent imaging), or infrared dyes for optical imaging, the antibody fragments can be used for preclinical imaging as shown herein and for clinical imaging in patients. These antigen binding constructs can also be used as potential SPECT imaging agents by simply changing the radiolabel to single photon emitting radionuclides such as Indium-111, Iodine-123, Technetium-99M, and Lutetium-177.

In some embodiments, the antigen binding constructs can be clinical imaging agents (PET/SPECT) in humans. Accordingly, in some embodiments, antigen binding constructs can be used for targeted diagnostic detection for these disorders. In some embodiments, the antigen binding construct can be used as a therapeutic.

### Definitions

All terms have their ordinary and customary meaning as understood by one of ordinary skill in the art, in view of the present disclosure.

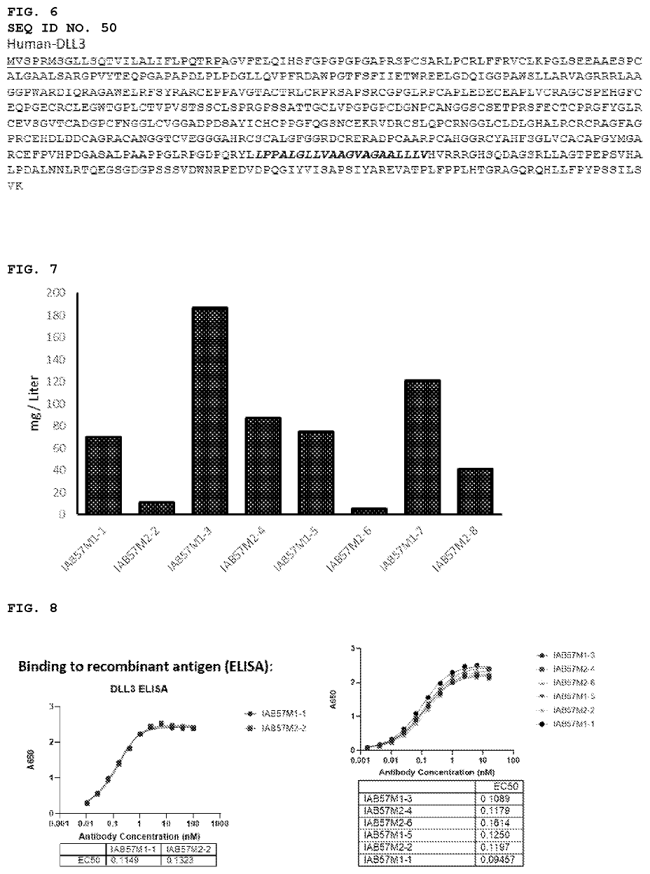

The term “antigen binding construct” includes all varieties of antibodies, including binding fragments thereof. Further included are constructs that include 1, 2, 3, 4, 5, and/or 6 CDRs. In some embodiments, tandem scFvs can be provided, which can provide two arms with bivalent binding. In some embodiments, these CDRs can be distributed between their appropriate framework regions in a traditional antibody. In some embodiments, the CDRs can be contained within a heavy and/or light chain variable region. In some embodiments, the CDRs can be within a heavy chain and/or a light chain. In some embodiments, the CDRs can be within a single peptide chain. Unless otherwise denoted herein, the antigen binding constructs described herein bind to the noted target molecule. The term “target” or “target molecule” denotes the protein to which the antigen binding construct binds. Examples of target proteins are known in the art, and include, for example DLL3 (FIG. 6; SEQ ID NO: 50).

The term “antibody” includes, but is not limited to, genetically engineered or otherwise modified forms of immunoglobulins, such as intrabodies, chimeric antibodies, fully human antibodies, humanized antibodies, antibody fragments, single-chain variable fragment (scFv), and heteroconjugate antibodies (for example, bispecific antibodies, diabodies, triabodies, tetrabodies, and nanobodies, etc.). The term “antibody” includes minibodies and diabodies. The term “antibody” includes a polypeptide of the immunoglobulin family or a polypeptide comprising fragments of an immunoglobulin that is capable of noncovalently, reversibly, and in a specific manner binding a corresponding antigen. An exemplary antibody structural unit comprises a tetramer. In some embodiments, a full length antibody can be composed of two identical pairs of polypeptide chains, each pair having one “light” and one “heavy” chain (connected through a disulfide bond). The recognized immunoglobulin genes include the kappa, lambda, alpha, gamma, delta, epsilon, hinge, and mu constant region genes, as well as the myriad immunoglobulin variable region genes. For full length chains, the light chains are classified as either kappa or lambda. For full length chains, the heavy chains are classified as gamma, mu, alpha, delta, or epsilon, which in turn define the immunoglobulin classes, IgG, IgM, IgA, IgD, and IgE, respectively. The N-terminus of each chain defines a variable region of up to about 149 or more amino acids primarily responsible for antigen recognition. The terms variable light chain (V_{L}) and variable heavy chain (V_{H}) refer to these regions of light and heavy chains respectively. As used in this application, an “antibody” encompasses all variations of antibody and fragments thereof. Also, the term “antibody” includes camelid derived immunoglobulins like single heavy-chain antibodies and Nanobodies®. Thus, within the scope of this concept are full length antibodies, chimeric antibodies, humanized antibodies, single chain antibodies (scFv), Fab, Fab′, and multimeric versions of these fragments (for example, F(ab′)₂) with the same binding specificity, scFv-Fc, single domain fragments (e.g., Nanobodies®), peptibodies, Nanobodies®, Nanobody®-Fc, minibodies, and diabodies. In some embodiments, the antibody binds specifically to a desired target.

The term “complementarity-determining domains” or “complementarity-determining regions (“CDRs”) interchangeably refer to the hypervariable regions of V_{L} and V_{H}. The CDRs are the target molecule-binding site of the antibody chains that harbors specificity for such target molecule. In some embodiments, there are three CDRs (CDR1-3, numbered sequentially from the N-terminus) in each V_{L} and/or V_{H}, constituting about 15-20% of the variable domains. The CDRs are structurally complementary to the epitope of the target molecule and are thus directly responsible for the binding specificity. The remaining stretches of the V_{L} or V_{H}, the so-called FRs, exhibit less variation in amino acid sequence (Kuby, Immunology, 4th ed., Chapter 4. W.H. Freeman & Co., New York, 2000).

The positions of the CDRs and framework regions can be determined using various well known definitions in the art, for example, Kabat (Wu, T. T. et al., “An analysis of the sequences of the variable regions of Bence Jones proteins and myeloma light chains and their implications for antibody complementarity,” J. Exp. Med., Vol. 132, No. 2, pp. 211-250, 1970; Kabat, E. A. et al., “Sequences of Proteins of Immunological Interest,” 5th Ed., NIH Publication No. 91-3242, Bethesda, MD, 1991); Chotia (Chothia C. et al., “Canonical structures for the hypervariable regions of immunoglobulins,” J. Mol. Biol., Vol. 196, No. 4, pp. 901-917, 1987; Chothia C. et al., “Conformations of immunoglobulin hypervariable regions,” Nature, Vol. 342, No. 6252, pp. 877-883, 1989; Chothia C. et al., “Structural repertoire of the human V_{H} segments,” J. Mol. Biol., Vol. 227, No. 3, pp. 799-817, 1992; Al-Lazikani B. et al., “Standard conformations for the canonical structures of immunoglobulins,” J. Mol. Biol., Vol. 273, No. 4, pp. 927-748, 1997), ImMunoGeneTics database (IMGT) (on the worldwide web at imgt.org/) (Giudicelli, V. et al., “IMGT/LIGM-DB, the IMGT® comprehensive database of immunoglobulin and T cell receptor nucleotide sequences,” Nucleic Acids Res., Vol. 34 (Database Issue), pp. D781-D784, 2006; Lefranc, M. P. et al., “IMGT unique numbering for immunoglobulin and T cell receptor variable domains and Ig superfamily V-like domains,” Dev. Comp. Immunol., Vol. 27, No. 1, pp. 55-77, 2003; Brochet, X. et al., “IMGT/V-QUEST: the highly customized and integrated system for IG and TR standardized V-J and V-D-J sequence analysis,” Nucleic Acids Res., Vol. 36 (Web Server Issue), pp. W503-508, 2008); AbM (Martin, A. C. et al., “Modeling antibody hypervariable loops: a combined algorithm,” Proc. Natl. Acad. Sci. U.S.A., Vol. 86, No. 23, pp. 9268-9272, 1989); AHo (Honegger A, Pluckthun A. Yet another numbering scheme for immunoglobulin variable domains: an automatic modeling and analysis tool. J Mol Biol. (2001) 309:657-70); Gelfand (Gelfand I M, Kister a E. Analysis of the relation between the sequence and secondary and three-dimensional structures of immunoglobulin molecules. Proc Natl Acad Sci USA. (1995) 92:10884-8); North (North B, Lehmann A, Dunbrack RLJ. A new clustering of antibody CDR loop conformations. J Mol Biol. (2011) 406:228-56); the contact definition (MacCallum, R. M. et al., “Antibody-antigen interactions: contact analysis and binding site topography,” J. Mol. Biol., Vol. 262, No. 5, pp. 732-745, 1996), and/or the automatic modeling and analysis tool (Honegger, A. et al., Accessible on the world wide web at bioc.uzh.ch/plueckthun/antibody/Numbering/). In some embodiments, the polypeptide is numbered from the beginning of the polypeptide signal sequence. In some embodiments, the polypeptide is numbered according from the beginning of the polypeptide and not including the signal sequence.

An “antibody variable light chain” or an “antibody variable heavy chain” as used herein refers to a polypeptide comprising the V_{L} or V_{H}, respectively. The endogenous V_{L} is encoded by the gene segments V (variable) and J (junctional), and the endogenous V_{H} by V, D (diversity), and J. Each of V_{L} or V_{H} includes the CDRs as well as the framework regions. In this application, antibody variable light chains and/or antibody variable heavy chains may, from time to time, be collectively referred to as “antibody chains.” These terms encompass antibody chains containing mutations that do not disrupt the basic structure of V_{L} or V_{H}, as one skilled in the art will readily recognize. In some embodiments, full length heavy and/or light chains are contemplated. In some embodiments, only the variable region of the heavy and/or light chains are contemplated as being present.

The term “hinge” denotes at least a part of a hinge region for an antigen binding construct, such as an antibody, a minibody, a scFv-Fc, or a Nanobody®-Fc. A hinge region can include a combination of the upper hinge, core (or middle) hinge and lower hinge regions. In some embodiments, the hinge is defined according to any of the antibody hinge definitions. Native IgG1, IgG2, and IgG4 antibodies have hinge regions having of 12-15 amino acids. IgG3 has an extended hinge region, having 62 amino acids, including 21 prolines and 11 cysteines. The functional hinge region of naturally occurring antibodies, deduced from crystallographic studies, extends from amino acid residues 216-237 of the IgG1 H chain (EU numbering; ref. 12) and includes a small segment of the N terminus of the CH2 domain in the lower hinge, with the lower hinge being the N terminus of CH2 domain. The hinge can be divided into three regions; the “upper hinge,” the “core,” and the “lower hinge”.

The term “artificial” or “non-natural” when modifying a hinge (or a subpart thereof) denotes that the sequence in question is not present, in the noted state, in nature. In the present context the hinges have been altered from their native state, so that their sequences are no longer those found in wild-type antibodies. As will be appreciated by those of skill in the art, minibodies do not naturally occur in nature, and thus, any construct which is a minibody construct is also not found in nature. This also applies to at least some of the constructs found in and/or incorporating the sequences of any of the hinge sequence tables provided herein (for example, Table 4). In some embodiments, any of the hinge subparts or full hinge sequences can be artificial hinge sequences, as long as the sequence (or resulting combination for the hinge) does not occur in nature.

The term “full hinge region” or “entire hinge region” denotes the presence of the entire upper, core, and lower hinge regions as a single construct. The upper, core, and lower regions can be positioned immediately adjacent to one another, or additional residues can be added between, or N- or C-terminal to the regions. In some embodiments, the native lower hinge can be replaced with an extension sequence. In some embodiments, one can combine a native lower hinge with the extension sequence. In some embodiments, an extension or other set of sequences can be added after the upper and/or core sequences.

The phrase “effective hinge region” denotes that an adequate amount of part of at least one of the upper, core and lower hinge regions is present to allow the hinge region to be effective for its intended purpose. Thus, the phrase encompasses variants of hinge regions and fragments of the various hinge regions. In some embodiments, the function of the hinge region is one or more of the following: to link the scFv with the C_{H}3 domain, provide flexibility and spacing for the two scFvs to bind to the target properly, to link two half molecules together, to provide overall stability to the molecule, and/or to provide a site for site-specific conjugation due to its solvent exposure. In some embodiments, the hinge should be close to natural as to reduce potential immunogenicity. In some embodiments, the upper hinge provides flexibility to scFv (starts at residue 216 in native IgGs), the middle hinge provides stability, and the lower hinge mediates flexibility to C_{H}3 (starts at residue 231 in native IgGs).

The term “upper hinge” denotes the first part of the hinge that starts at the end of the scFv. The upper hinge includes the amino acids from the end of the scFv up to, but not including, the first cysteine residue in the core hinge. As above, the term “effective upper hinge” denotes that enough of the sequence is present to allow the section to function as an upper hinge; the term encompasses functional variants and fragments of the designated hinge section.

The term “core hinge” denotes the second part of the hinge region that is C-terminal to the upper hinge. The core hinge contains the inter-chain disulfide bridges and a high content of prolines. As above, the term “effective core hinge” denotes that enough of the sequence is present to allow the section to function as a core hinge; the term encompasses functional variants and fragments of the designated hinge section.

The term “lower hinge” denotes the third part of the hinge region that is C terminal to the core hinge. In the context of a minibody or antibody fragment, the lower hinge connects to the C_{H}3 domain. As above, the term “effective lower hinge” denotes that enough of the sequence is present to allow the section to function as a lower hinge; the term encompasses functional variants and fragments of the designated hinge section. The term “lower hinge” as used herein can encompass various amino acid sequences including naturally occurring IgG lower hinge sequences and artificial extension sequences in place of one another or a combination thereof provided herein. In some embodiments, the various extensions can be considered to be a lower hinge region in its entirety or a replacement.

Antibodies can exist as intact immunoglobulins or as a number of fragments produced by digestion with various peptidases. Thus, for example, pepsin digests an antibody below the disulfide linkages in the hinge region to produce F(ab)′₂, a dimer of Fab′ which itself is a light chain (V_{L}-C_{L}) joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is a Fab with part of the hinge region. (Paul, W. E., “Fundamental Immunology,” 3d Ed., New York: Raven Press, 1993). While various antibody fragments are defined in terms of the digestion of an intact antibody, one of skill will appreciate that such fragments may be synthesized de novo either chemically or by using recombinant DNA methodology. Thus, the term “antibody,” as used herein, also includes antibody fragments either produced by the modification of whole antibodies, or those synthesized de novo using recombinant DNA methodologies (for example, single chain Fv) or those identified using phage display libraries (see, for example, McCafferty, J. et al., “Phage antibodies: filamentous phage displaying antibody variable domains,” Nature, Vol. 348, No. 66301, pp. 552-554, 1990). For preparation of monoclonal or polyclonal antibodies, any technique known in the art can be used (see, for example, Kohler, G. et al., “Continuous cultures of fused cells secreting antibody of predefined specificity,” Nature, Vol. 256, No. 5517, pp. 495-497, 1975; Kozbor, D. et al., “The production of monoclonal antibodies from human lymphocytes,” Immunology Today, Vol. 4, No. 3, pp. 72-79, 1983; Cole, et al., “Monoclonal Antibodies and Cancer Therapy,” Alan R. Liss, Inc., pp. 77-96, 1985; Wang, S., “Advances in the production of human monoclonal antibodies,” Antibody Technology Journal, Vol. 1, pp. 1-4, 2011; Sharon, J. et al., “Recombinant polyclonal antibodies for cancer therapy,” J. Cell Biochem., Vol. 96, No. 2, pp. 305-313, 2005; Haurum, J. S., “Recombinant polyclonal antibodies: the next generation of antibody therapeutics?,” Drug Discov. Today, Vol. 11, No. 13-14, pp. 655-660, 2006). Techniques for the production of single chain antibodies (U.S. Pat. No. 4,946,778) can be adapted to produce antibodies to polypeptides of the present disclosure. Also, transgenic mice, or other organisms such as other mammals, may be used to express fully human monoclonal antibodies. Furthermore, E. Coli or yeast may be used to express and manufacture recombinant antibodies and antibody fragments (Simmons L. C., Reilly D., Klimowski L., Shantha Raju T., Meng G., Sims P., Hong K., Shields R. L., Damico L. A., Rancatore P., Yansura D. G.; Expression of full-length immunoglobulins in Escherichia coli: rapid and efficient production of aglycosylated antibodies, J Immunol Methods. 2002 May 1; 263(1-2):133-47; Kulagina N, Besseau S, Godon C, Goldman G. H., Papon N., Courdavault V., Yeasts as biopharmaceutical production platforms; Front. Fungal Biol., 22 Sep. 2021). Alternatively, phage display and yeast display technologies can be used to identify high affinity binders to selected antigens (see, for example, McCafferty et al., supra; Marks, J. D. et al., “By-passing immunization: building high affinity human antibodies by chain shuffling,” Biotechnology (N. Y.), Vol. 10, No. 7, pp. 779-783, 1992; Feldhaus M. J., Siegel R. W., Yeast display of antibody fragments: a discovery and characterization platform, J Immunol Methods, 2004 July; 290(1-2):69-80). Alternatively, antibodies can be produced through B-cell screening technologies from human hosts (Pedrioli A., Oxenius A., Single B cell technologies for monoclonal antibody discovery, Trends in Immunology, (2021) volume 42, issue 12, p 1143-1158). Furthermore, antibodies can be derived from immunization of camelid animals or screening of camelid phage libraries (Harmsen M. M., De Haard H. J., Properties, production, and applications of camelid single-domain antibody fragments, Appl Microbiol Biotechnol. 2007; 77(1): 13-22). Alternatively, antibodies can be derived from in-silico screening simulations through deep learning and artificial intelligence algorithms (Graves J., Byerly J., Priego E., Makkapati N., Vince Parish S., Brenda Medellin and Monica Berrondo, A Review of Deep Learning Methods for Antibodies, Antibodies 2020, 9, 12).

Methods for humanizing or primatizing non-human antibodies are well known in the art. Generally, a humanized antibody has one or more amino acid residues introduced into it from a source which is non-human. These non-human amino acid residues are often referred to as import residues, which are typically taken from an import variable domain. In some embodiments, the terms “donor” and “acceptor” sequences can be employed. Humanization can be essentially performed following the method of Winter and co-workers (see, for example, Jones, P. T. et al., “Replacing the complementarity-determining regions in a human antibody with those from a mouse,” Nature, Vol. 321, No. 6069, pp. 522-525, 1986; Riechmann, L. et al., “Reshaping human antibodies for therapy,” Nature, Vol. 332, No. 6162, pp. 323-327, 1988; Verhoeyen, M. et al., “Reshaping human antibodies: grafting an antilysozyme activity,” Science, Vol. 239, No. 4847, pp. 1534-1536, 1988; Presta, L. G., “Antibody engineering,”, Curr. Op. Struct. Biol., Vol. 2, No. 4, pp. 593-596, 1992), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some complementarity determining region (“CDR”) residues and possibly some framework (“FR”) residues are substituted by residues from analogous sites in rodent antibodies.

The term “Fe region” or “Fc domain” or “Fc” denotes a C-terminal region of an immunoglobulin heavy chain. The “Fe region” may be a native sequence Fe region or a variant Fc region (e.g., a variant having one or more mutations that reduces an effector function, e.g., FcγR binding, and/or binding to the Fc neonatal receptor (FcRn), etc.). The Fc region of an immunoglobulin (e.g., IgG) generally comprises two constant domains, C_{H}2 and C_{H}3. The Fc region can include a hinge region or sequence, as described herein. An Fc region can be present in dimer or monomeric form. In some embodiments, the Fe region is a human Fc region, or a variant thereof.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, for example, an enzyme, toxin, hormone, growth factor, and drug; or (b) the variable region, or a portion thereof, is altered, replaced or exchanged with a variable region having a different or altered antigen specificity.

The term “antibody fragment” includes, but is not limited to one or more antigen binding fragments of antibodies alone or in combination with other molecules, including, but not limited to Fab′, F(ab′)₂, Fab, Fv, rIgG (reduced IgG), scFv fragments, scFv-Fc, single domain fragments (e.g., Nanobodies® or single domain fragments), peptibodies, Nanobodies®, Nanobody®-Fe, minibodies, and diabodies. The term “scFv” refers to a single chain Fv (“fragment variable”) antibody in which the variable domains of the heavy chain and of the light chain of a traditional two chain antibody have been joined to form one chain.

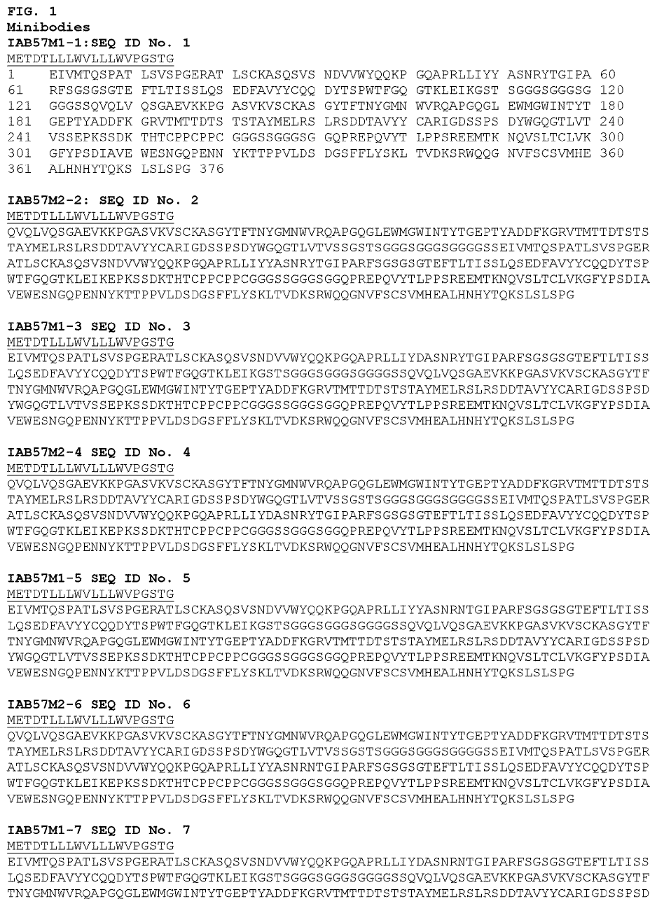

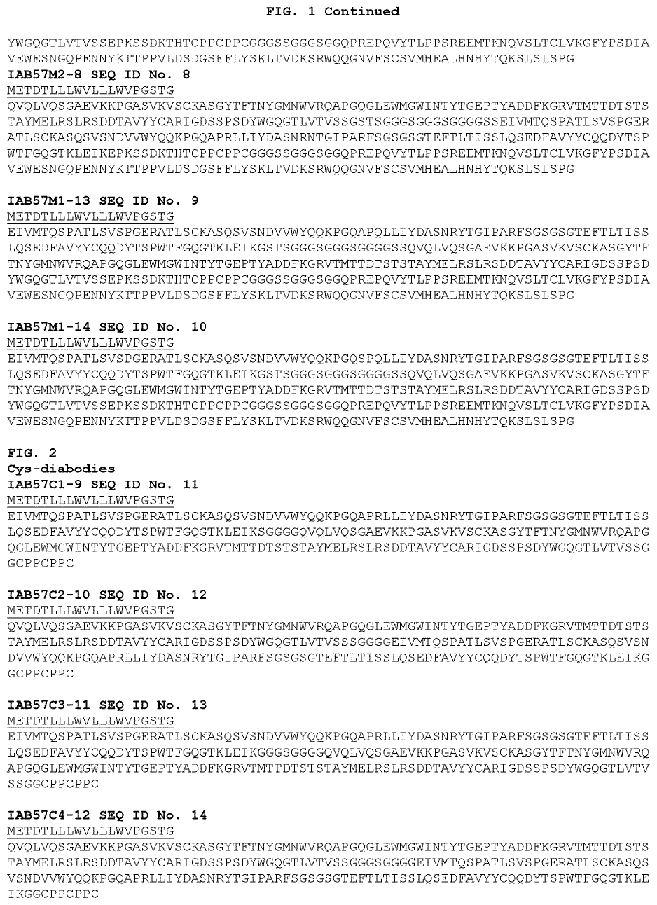

The term “artificial” or “non-natural” when modifying a CDR or FR (or a subpart thereof) denotes that the sequence in question is not present, in the noted state, in nature. In the present context the CDRs or FRs have been altered from their native state, so that their sequences are no longer those found in wild-type antibodies. As will be appreciated by those of skill in the art, minibodies and cys-diabodies do not naturally occur in nature, and thus, any construct which is a minibody or a cys-diabody construct is also not found in nature. This also applies to at least some of the constructs found in and/or incorporating the sequences of any of the CDR or FR sequence tables provided herein. In some embodiments, any of the CDR or FR sequences in the figures or tables, for example in FIG. 1, Table 1, or Table 2, can be artificial CDR or FR sequences, as long as the sequence (or resulting combination for the CDR or FR) does not occur in nature.

A “minibody” is an antibody format that has a smaller molecular weight than the full-length antibody while maintaining the bivalent binding property against an antigen. Because of its smaller size, absence of CH2 domain that binds Fc-gamma and FcRn receptors, absence of glycosylation, the minibody has a faster clearance from the system and potentially enhanced penetration when targeting tumor tissue. With the ability for strong targeting combined with rapid clearance, the minibody is advantageous for diagnostic imaging and delivery of radioactive payloads for which prolonged circulation times may result in adverse patient dosing or dosimetry. In some embodiments, it can also be advantageous for delivery of a cytotoxic payload due to the above-mentioned features such as tumor penetration and faster clearance. A “minibody” as described herein, encompasses a homodimer, wherein each monomer is a single-chain variable fragment (scFv) linked to a human IgG C_{H}3 domain by a hinge sequence. In some embodiments, a minibody is a bivalent or bispecific, covalently bound homodimer of ~80 kDa. In some embodiments, each monomer (half-molecule) is comprised of a variable heavy (V_{H}) domain linked to the corresponding variable light (V_{L}) domain by an approximate 15-18 amino acid Gly-Ser-rich linker sequence.

A “diabody” comprises a first polypeptide chain which comprises a heavy chain variable domain (V_{H}) connected to a light chain variable domain (V_{L}) on the first polypeptide chain (V_{H}-V_{L} or V_{L}-V_{H}) connected by a peptide linker that does not allow pairing between the two domains on the first polypeptide chain (e.g., the peptide linker is too short to allow the pairing), and a second polypeptide chain comprising a heavy chain variable domain (V_{H}) linked to a light chain variable domain (V_{L}) on the second polypeptide chain (V_{H}-V_{L} or V_{L}-V_{H}) connected by a peptide linker that does not allow pairing between the two domains on the second polypeptide chain (e.g., the peptide linker is too short to allow the pairing). Without being limited by theory, the short linkages can force chain pairing between the complementary domains of the first and the second polypeptide chains and promotes the assembly of a dimeric molecule with two functional antigen binding sites. Therefore, a peptide linker may be any suitable length that promotes such assembly, for example, between 5 and 20 amino acids in length. A “cys-diabody” denotes a diabody whose monomer chains are covalently linked by a disulfide bond. In some embodiments, a “cys-diabody” is a diabody with one or more than one C-terminal cysteines.

The term “extension sequence” (e.g., in a diabody context) denotes a region that connects a first V_{H} domain to a second V_{H} domain, or a first V_{L} to a second V_{L} domain, in for example, a diabody. Extension sequences can connect the domains through the C-terminus of each domain. In some embodiments, extension sequences connect the domains through covalent bonds. In some embodiments, the extension sequence will include one or more cysteine, allowing for one or more disulfide bonds to be formed between two such extension sequences. A non-limiting example of an extension sequence includes -(Gly)₂-(Cys). In some embodiments, the extension sequence includes 1, 2, 3, or more cysteines per monomer chain. While the extension sequence will be towards the C-terminus of the constructs, it need not be the absolute last amino acid in the variable domain. That is, the extension sequence can be positioned slightly N-terminal to the C-terminus. For example, the extension sequence can be placed within the 10 amino acids of the C-terminus of the monomer. Similarly, additional sequence can be placed between the native C-terminus and where the extension sequence starts. The extension sequence can connect V_{H} to V_{H} or V_{L} to V_{L} through a disulfide bond. In some embodiments, the extension sequence includes GGCPPCPPC (SEQ ID NO: 93).

As used herein, “pharmaceutically acceptable” has its plain and ordinary meaning as understood in light of the specification and refers to carriers, excipients, and/or stabilizers that are nontoxic to the cell or mammal being exposed thereto at the dosages and concentrations employed or that have an acceptable level of toxicity. A “pharmaceutically acceptable” “diluent,” “excipient,” and/or “carrier” as used herein have their plain and ordinary meaning as understood in light of the specification and are intended to include any and all solvents, dispersion media, coatings, antibacterial or antifungal agents, isotonic or absorption delaying agents, compatible with administration to humans, primates, cats, dogs, or other vertebrate hosts. Typically, a pharmaceutically acceptable diluent, excipient, and/or carrier is a diluent, excipient, and/or carrier approved by a regulatory agency of a Federal, a state government, or other regulatory agency, or listed in the U.S. Pharmacopeia or other generally recognized pharmacopeia for use in animals, including humans as well as non-human mammals, such as cats and dogs. The term diluent, excipient, and/or “carrier” can refer to a diluent, adjuvant, excipient, or vehicle with which the pharmaceutical composition is administered. Such pharmaceutical diluent, excipient, and/or carriers, which can be incorporated in any one or more of the compositions described herein, include sterile liquids, such as water and oils, including those of petroleum, animal, vegetable or synthetic origin. Water, saline solutions or aqueous dextrose and glycerol solutions can be employed as liquid diluents, excipients, and/or carriers. Suitable pharmaceutical diluents and/or excipients, which can be incorporated in any one or more of the compositions described herein, also include starch, glucose, lactose, sucrose, gelatin, malt, rice, flour, chalk, silica gel, sodium stearate, glycerol monostearate, talc, sodium chloride, dried skim milk, glycerol, propylene, glycol, water, or ethanol. The physiologically acceptable carrier may also comprise one or more of the following: antioxidants, such as ascorbic acid, low molecular weight (less than about 10 residues) polypeptides, proteins, such as serum albumin, gelatin, immunoglobulins, hydrophilic polymers such as polyvinylpyrrolidone, amino acids, carbohydrates such as glucose, mannose, or dextrins, chelating agents such as EDTA, sugar alcohols such as mannitol or sorbitol, salt-forming counterions such as sodium, and nonionic surfactants such as TWEEN®, polyethylene glycol (PEG), PLURONICS® or preservatives such as an essential oil, methyl paraben, propyl paraben, or sodium salt of parabens. In some embodiments, the preservative is bronidiol. The composition, if desired, can also contain minor amounts of wetting, bulking, emulsifying agents, or pH buffering agents. These compositions can take the form of solutions, suspensions, emulsion, sustained release formulations and the like. The formulation should suit the mode of administration.

Additional excipients with desirable properties include but are not limited to preservatives, adjuvants, stabilizers, solvents, buffers, diluents, solubilizing agents, detergents, surfactants, chelating agents, antioxidants, alcohols, ketones, aldehydes, ethylenediaminetetraacetic acid (EDTA), citric acid, salts, sodium chloride, sodium bicarbonate, sodium phosphate, sodium borate, sodium citrate, potassium chloride, potassium phosphate, magnesium sulfate sugars, dextrose, fructose, mannose, lactose, galactose, sucrose, sorbitol, cellulose, serum, amino acids, polysorbate 20, polysorbate 80, sodium deoxycholate, sodium taurodeoxycholate, magnesium stearate, octylphenol ethoxylate, benzethonium chloride, thimerosal, gelatin, esters, ethers, 2-phenoxyethanol, urea, or vitamins, or any combination thereof. In some embodiments, the formulation includes an at least one agent that acts to reduce radiolysis (also known as “radioprotectors”) or is a kidney protecting agent. Non-limiting examples of radiolysis reducing agents include Gentisic acid, Acetylcholine, AET, ACE inhibitors, acteoside, alpha-tocopherol acetate, amifostine, ascorbic acid, aspirin, atorvastatin, beta-carotene, Bowman-Birk proteinase inhibitor, Caffeic acid, Captopril, carbaminoylcholine, Carvacrol, Celecoxib, coenzyme Q10, COX2 inhibitors/NSAIDs, curcumin, cysteine, cysteamine, cystamine, dendrodine analog, Dithiolthione, Dopamine, enalapril, epigallocatechin-3-gallate, Epinephrine, 17-β-estradiol, GANRA-5, Genistein, green tea abstract, growth factors, guanine nucleotides, Halofuginone, Hmg-CoA reductase inhibitors (statins), heroin, histamine, ibuprofen, inapoyl-E-glucoside, Isoflavone, isofraxidin, kukoamine A, lactoferrin amifostine, lipoic acid, lovastatin, luteolin-7-O-(2-apiosyl)-glucoside, 2-mercaptoethylguanidine, melatonin, methacholine, morphine, N-acetyl cysteine, Oltipraz, palifermin, phenethyl ester, polyphenols, pravastatin, protease inhibitors, quercetin-3-O-rhamnoside-7-O-glucoside, quercetin-3-O-rhamnoside, ramipril, Resveratrol, rutin, serotonin, Simvastatin, Sodium ascorbate, superoxide dismutase, TGF-signaling inhibitors, tocopherols, vitamin C, vitamin E, watermelon juice, black grape juice, and thiols such as glutathione. Kidney protecting agents include free lysine, arginine, probenecid, gelofusin, and other compositions. Some excipients may be in residual amounts or contaminants from the process of manufacturing, including but not limited to serum, albumin, ovalbumin, antibiotics, inactivating agents, formaldehyde, glutaraldehyde, β-propiolactone, gelatin, cell debris, nucleic acids, peptides, amino acids, or growth medium components or any combination thereof. The amount of the excipient may be found in the composition at a percentage that is, is about, is at least, is at least about, is not more than, or is not more than about, 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 95%, 100% w/w or any percentage by weight in a range defined by any two of the aforementioned numbers.

As used herein, a “carrier” has its plain and ordinary meaning as understood in light of the specification and refers to a compound, particle, solid, semi-solid, liquid, or diluent that facilitates the passage, delivery and/or incorporation of a compound to cells, tissues and/or bodily organs.

As used herein, a “diluent” has its plain and ordinary meaning as understood in light of the specification and refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

The term “target molecule dependent disorder” or “target molecule associated disorder” includes any disorder in which the target molecule plays a role in the disorder itself. In some embodiments, this denotes over-expression of the target molecule. In some embodiments, the disorders can include any of the disorders discussed herein. In some embodiments, the disorder can be any for which there is a target molecule that can be targeted by binding, whose binding will result in the detection and/or treatment of the disorder.

The term “treating” or “treatment” of a condition can refer to preventing the condition, slowing the onset and/or rate of development of the condition, reducing the risk of developing the condition, preventing and/or delaying the development of symptoms associated with the condition, reducing or ending symptoms associated with the condition, generating a complete or partial regression of the condition, or some combination thereof. The term “prevent” does not require the absolute prohibition of the disorder or disease. Examples of disorders or diseases include fibrosis, cancer, tumor and neoplasms, autoimmune disease, cardiovascular, neurodegenerative, metabolic and endocrine disorders, inflammatory, immunity, genetic disorders, infectious disorders, hematological, congenital disorders, musculoskeletal, oral and gastrointestinal, renal and urogenital disorders, reproductive disorders, respiratory disorders, skin and/or epithelial, as well as disorders with disputed or unknown etiology.

“Tumor,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumor” are not mutually exclusive as referred to herein. The term “neoplasia” encompasses the term tumor.

The terms “cancer” and “cancerous” refer to or describe the physiological condition in mammals that is typically characterized by unregulated cell growth. Examples of cancer include, but are not limited to, carcinoma, lymphoma, blastoma, sarcoma, and leukemia or lymphoid malignancies. More particular examples of such cancers include lung cancer including small-cell lung cancer, non-small cell lung cancer and lung adenocarcinomas with neuroendocrine features; neuroendocrine prostate cancer, melanoma, gliomas, low-grade gliomas and glioblastoma, medullary thyroid cancer, carcinoid tumors, neuroendocrine tumors in the pancreas, bladder cancer, testicular cancer squamous cell cancer (e.g. epithelial squamous cell cancer), neuroendocrine neoplasms, such as neuroendocrine tumors of unknown primary, neuroendocrine neoplasms of the small bowel, carotid body, adrenal gland, colorectal gynecological organ, abdomen, esophagus, GI tract, bile duct, nervous system, appendix, liver, anal, thymus, ileocecal junction, head and neck, breast, peritoneum and retroperitoneum, kidney, thyroid, stomach, bone; adenocarcinomas, such as adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, bone cancer, hepatic carcinoma, anal carcinoma, penile carcinoma, melanoma, multiple myeloma and B-cell lymphoma, brain, as well as head and neck cancer, and associated metastases. The term cancer includes adult and pediatric solid cancers. In some embodiments, the cancer can be a solid tumor. In some embodiments, the cancer is a highly fibrotic tumor or cancer. In some embodiments, the cancer is a desmoplasia.

A “therapeutically effective amount” or a “therapeutically effective dose” is an amount that produces a desired therapeutic effect in a subject, such as preventing, treating a target condition, delaying the onset of the disorder and/or symptoms, and/or alleviating symptoms associated with the condition. This amount will vary depending upon a variety of factors, including but not limited to the characteristics of the therapeutic compound (including activity, pharmacokinetics, pharmacodynamics, biodistribution and bioavailability), the physiological condition of the subject (including age, sex, disease type and stage, general physical condition, responsiveness to a given dosage, and type of medication), the nature of the pharmaceutically acceptable carrier or carriers in the formulation, and/or the route of administration. One skilled in the clinical and pharmacological arts will be able to determine a therapeutically effective amount through routine experimentation, for example by monitoring a subject's response to administration of a compound and adjusting the dosage accordingly, given the present disclosure. For additional guidance, see Remington: The Science and Practice of Pharmacy 21^{st} Edition, Univ. of Sciences in Philadelphia (USIP), Lippincott Williams & Wilkins, Philadelphia, PA, 2005.

“Label”, “detectable label” or “detectable marker” are used interchangeably herein and refer to a detectable compound or composition which is conjugated directly or indirectly associated with the antibody so as to generate a “labeled” antibody. The label may be detectable by itself (e.g., radioisotope labels or fluorescent labels) or, in the case of an enzymatic label, may catalyze chemical alteration of a substrate compound or composition which is detectable.

The term “payload” denotes an atom or molecule or other entity that is associated (covalently or otherwise) to an antigen binding construct. It includes labels or markers for aspects for diagnostics for example, as well as toxins, cytotoxic agents, chemotherapeutic agents for various therapies. In some embodiments, the payload involves as chelator so as to attach the antigen binding construct to the molecule or atom to be delivered via or colocalized via the antigen binding construct.

The term “cytotoxic agent” as used herein refers to a substance that inhibits or prevents a cellular function and/or causes cell death or destruction. The term is intended to include non-radioactive isotopes (ADC), radioactive isotopes (e.g., ¹⁷⁷Lu, ²²⁵Ac, ⁶⁷Cu, ²²⁷Th, ²¹¹At, ¹³¹I, ¹²⁵I, ⁹⁰Y, ¹⁸⁶Re, ¹⁸⁸Re, ¹⁵³Sm, ²¹²Bi, ²¹³Bi, ³²P, ¹⁴⁹Tb, ¹⁶¹Tb, ²¹²Pb, and radioactive isotopes of Lu), chemotherapeutic agents (as defined elsewhere herein). Other cytotoxic agents are described below. A tumoricidal agent causes destruction of tumor cells.

A “toxin” is any substance capable of having a detrimental effect on the growth or proliferation of a cell. Non-radioactive payloads include those commonly used for antibody drug conjugates (ADC) and fragment drug conjugates (FDC), such as toxins belonging to the families of auristatins, maytansines, maytansinoids, calicheamicins, duocarymycins, pyrrolobenzodiazepines dimers and amatoxins.

A “therapeutic ion” refers to an electrically charged particle that that is useful in the treatment of a disorder related to a target molecule. Examples of therapeutic ions include ¹⁸F, ¹⁸F-FAC, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁵Sc, ⁷⁷As, ⁸⁶Y, ⁹⁰Y, ⁸⁹Sr, ⁸⁹Zr, ⁹⁴Tc, ⁹⁴Tc, ⁹⁹mTc, ⁹⁹Mo, ¹⁰⁵Pd, ¹⁰⁵P, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁴⁹Tb, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ⁷⁷Lu, ¹⁸⁶Re, ¹⁸⁸Re, ¹⁸⁹Re, ¹⁹⁴I, ¹⁹⁸Au, ¹⁹⁹Au, ²¹¹At, ²¹¹Pb, ²¹²Bi, ²¹²Pb, ²¹³Bi, ²²³Ra, ²²⁷Th, and ²²⁵Ac. These are also options of therapeutic agents.

A “chemotherapeutic agent” is a chemical compound useful in the treatment of cancer. Examples of chemotherapeutic agents include alkylating agents such as thiotepa and CYTOXAN™ cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylolomelamine; acetogenins (especially bullatacin and bullatacinone); delta-9-tetrahydrocannabinol (dronabinol, MARINOL™); beta-lapachone; lapachol; colchicines; betulinic acid; a camptothecin (including the synthetic analogue topotecan (HYCAMTIN™), CPT-11 (irinotecan, CAMPTOSAR™), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calichcamicin, especially calicheamicin gammall and calicheamicin omegall (see, e.g., Agnew, Chem Intl. Ed. Engl., 33: 183-186 (1994)); dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycins, dactinomycin, daunorubicin, detorubicin, 6-diazo-5-oxo-L-norleucine, ADRIAMYCIN™ doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; metabolic inhibitor such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE™, FILDESIN™); dacarbazine; mannomustine; mitobronitol; mitolactol; pipobroman; gacytosine; arabinoside (“Ara-C”); thiotepa; taxoids, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE™ docetaxel (Rhone-Poulenc Rorer, Antony, France); chloranbucil; gemcitabine (GEMZAR™); 6-thioguanine; mercaptopurine; methotrexate; platinum analogs such as cisplatin and carboplatin; vinblastine (VELBAN™); platinum; etoposide (VP-16); ifosfamide; mitoxantrone; vincristine (ONCOVIN™); oxaliplatin; leucovovin; vinorelbine (NAVELBINE™); novantrone; edatrexate; daunomycin; aminopterin; ibandronate; topoisomerase inhibitor RFS 2000; difluoromethylornithine (DMFO); retinoids such as retinoic acid; capecitabine (XELODA™); pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin. These are also options of therapeutic agents.

“Radiotherapy” means treatment using radiation or a radio-isotope with a therapeutic purpose. It includes radiation therapy intended to have abscopal effect as described in Yang Liu, Yinping Dong, Li Kong, Fang Shi, Hui Zhu & Jinming Yu; “Abscopal effect of radiotherapy combined with immune checkpoint inhibitors”; Journal of Hematology & Oncology volume 11, Article number: 104 (2018); and in Melek Tugce Yilmaz, Aysenur Elmali, and Gozde Yazici; “Abscopal Effect, From Myth to Reality: From Radiation Oncologists' Perspective”; Cureus. 2019 January; 11(1).

The terms “subject,” “patient,” and “individual” interchangeably refer to an entity that is being examined and/or treated. The term “mammal” is used in its usual biological sense. Thus, it specifically includes, but is not limited to, primates, including simians (chimpanzees, apes, monkeys), humans, cattle, horses, sheep, goats, swine, rabbits, dogs, cats, rodents, rats, mice, or guinea pigs.

The term “co-administer” refers to the administration of two active agents in the blood of an individual or in a sample to be tested. Active agents that are co-administered in combination or sequentially delivered. “In combination” means that two (or more) different compositions are delivered to the subject during the course of the subject's affliction with the disorder, e.g., the two or more compositions are delivered after the subject has been diagnosed or selected as one having the disorder and before the disorder has been cured or eliminated. In some embodiments the subject is selected to receive any one or more of the compositions described herein by diagnostic analysis or clinical evaluation or both. In some embodiments, the delivery of one therapy is still occurring when the delivery of the second begins, so that there is overlap. This is sometimes referred to herein as “simultaneous” or “concomitant” or “concurrent delivery”. In other embodiments, the delivery of one therapy ends before the delivery of the other therapy begins. This is sometimes referred to herein as “successive” or “sequential delivery.” In embodiments of either case, the therapy is more effective because of combined administration. For example, the second therapy is a more effective, e.g., an equivalent effect is seen with less of the second therapy, or the second therapy reduces symptoms to a greater extent, than would be seen if the second therapy were administered in the absence of the first therapy, or the analogous situation is seen with the first therapy. In some embodiments, delivery is such that the reduction in a symptom, or other parameter related to the disorder is greater than what would be observed with one therapy delivered in the absence of the other. The effect of the two therapies can be partially additive, wholly additive, or greater than additive (e.g., synergistic). The delivery can be such that an effect of the first therapy delivered is still detectable when the second is delivered.

The phrase “specifically (or selectively) bind,” when used in the context of describing the interaction between an antigen, for example, a protein, to an antibody or antibody-derived binding agent, refers to a binding reaction that is determinative of the presence of the antigen in a heterogeneous population of proteins and other biologics, for example, in a biological sample, for example, a blood, serum, plasma or tissue sample. Thus, under designated immunoassay conditions, in some embodiments, the antibodies or binding agents with a particular binding specificity bind to a particular antigen at least two times the background and do not substantially bind in a significant amount to other antigens present in the sample. Specific binding to an antibody or binding agent under such conditions may require the antibody or agent to have been selected for its specificity for a particular protein. A variety of immunoassay formats may be used to select antibodies specifically immunoreactive with a particular protein. For example, solid-phase ELISA immunoassays are routinely used to select antibodies specifically immunoreactive with a protein (see, for example, Harlow, E. & Lane D., “Using Antibodies, A Laboratory Manual,” Cold Spring Harbor Laboratory Press, 1998, for a description of immunoassay formats and conditions that can be used to determine specific immunoreactivity). Typically a specific or selective binding reaction will produce a signal at least twice over the background signal and more typically at least than 10 to 100 times over the background.

The term “equilibrium dissociation constant (K_{D}, M)” refers to the dissociation rate constant (k_{d}, time⁻¹) divided by the association rate constant (kₐ, time⁻¹ M⁻¹). Equilibrium dissociation constants can be measured using any known method in the art. The antibodies of the present disclosure generally will have an equilibrium dissociation constant of less (that is superior binding) than about 10⁻⁷ or 10⁻⁸ M, for example, less than about 10⁻⁹ M or 10⁻¹⁰ M, in some embodiments, less than about 10⁻¹¹ M, 10⁻¹² M, or 10⁻¹³ M.

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (for example, degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer, M. A. et al., “Enhanced evolutionary PCR using oligonucleotides with inosine at the 3′-terminus,” Nucleic Acid Res., Vol. 19, No. 18, pp. 5081, 1991; Ohtsuka, E. et al., “An alternative approach to deoxyoligonucleotides as hybridization probes by insertion of deoxyinosine at ambiguous codon positions,” J. Biol. Chem., Vol. 260, No. 5, pp. 2605-2608, 1985; Rossolini, G. M. et al., “Use of deoxyinosine-containing primers vs degenerate primers for polymerase chain reaction based on ambiguous sequence information,” Mol. Cell. Probes, Vol. 8, No. 2, pp. 91-98, 1994).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, for example, hydroxyproline, gamma-carboxyglutamate, and O-phosphoserine. Amino acid analogs refer to compounds that have the same basic chemical structure as a naturally occurring amino acid, for example, an alpha-carbon that is bound to a hydrogen, a carboxyl group, an amino group, and an R group, for example, homoserine, norleucine, methionine sulfoxide, methionine methyl sulfonium. Such analogs have modified R groups (for example, norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

The term “conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the present disclosure.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, for example, Creighton, T. E., “Proteins—Structures and Molecular Properties,” W. H. Freeman & Co. Ltd., 1984).

The term “percentage of sequence identity” can be determined by comparing two optimally aligned sequences over a comparison window, wherein the portion of the polynucleotide sequence in the comparison window may comprise additions or deletions (i.e., gaps) as compared to the reference sequence (for example, a polypeptide of the present disclosure), which does not comprise additions or deletions, for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same sequences. Two sequences are “substantially identical” if two sequences have a specified percentage of amino acid residues or nucleotides that are the same (for example, 70%, 75%, 80%, 85%, 90%, 95%, 96%, 97%, 98% or 99% sequence identity over a specified region, or, when not specified, over the entire sequence of a reference sequence), when compared and aligned for maximum correspondence over a comparison window, or designated region as measured using one of the following sequence comparison algorithms or by manual alignment and visual inspection. Some embodiments provided herein provide polypeptides or polynucleotides that are substantially identical to the polypeptides or polynucleotides, respectively, exemplified herein. Optionally, the identity exists over a region that is at least about 15, 25 or 50 nucleotides in length, or more preferably over a region that is 100 to 500 or 1000 or more nucleotides in length, or over the full length of the reference sequence. With respect to amino acid sequences, identity or substantial identity can exist over a region that is at least 5, 10, 15 or 20 amino acids in length, optionally at least about 25, 30, 35, 40, 50, 75 or 100 amino acids in length, optionally at least about 150, 200 or 250 amino acids in length, or over the full length of the reference sequence. With respect to shorter amino acid sequences, for example, amino acid sequences of 20 or fewer amino acids, in some embodiments, substantial identity exists when one or two amino acid residues are conservatively substituted, according to the conservative substitutions defined herein.

In some embodiments, the percent identity is over the CDR and/or FR regions noted herein. In such situations, the percent identity of the CDR or FR can be identified separately from the rest of the protein or nucleic acid sequence. Thus, two CDRs or FRs can have a specified percentage of amino acid residues or nucleotides that are the same (for example, 70%, 75%, 80%, 85%, 90%, 95%, 96%, 97%, 98% or 99% sequence identity over a specified region, or, when not specified, over the entire sequence of a reference sequence), while allowing for the remainder of the protein to either stay 100% identical to the comparison protein, our while also allowing the remainder of the protein to also have variation by a specified percent identity.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, for example, by the local homology algorithm of Smith and Waterman (1970) Adv. Appl. Math. 2:482c, by the homology alignment algorithm of Needleman, S. B. et al., “A general method applicable to the search for similarities in the amino acid sequence of two proteins,” J. Mol. Biol., Vol. 48, No. 3, pp. 443-453, 1970, by the search for similarity method of Pearson, W. R. et al., “Improved tools for biological sequence comparison,” Proc. Natl. Acad. Sci. U.S.A., Vol. 85, No. 8, pp. 2444-2448, 1988, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, for example, Ausubel, F. M. et al., Current Protocols in Molecular Biology, Supplement, 1995).

Two examples of algorithms that are suitable for determining percent sequence identity and sequence similarity are the BLAST and BLAST 2.0 algorithms, which are described in Altschul, S. F. et al., “Gapped BLAST and PSI-BLAST: a new generation of protein database search programs,” Nucleic Acids Res., Vol. 25, No. 17, pp. 3389-3402, 1977, and Altschul, S. F. et al., “Basic local alignment search tool,” J. Mol. Biol., Vol. 215, No. 3, pp. 403-410, 1990, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul, S. F. et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues: always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see, Henikoff, S. et al., “Amino acid substitution matrices from protein blocks,” Proc. Natl. Acad. Sci. U.S.A., Vol. 89, No. 22, pp. 10915-10919, 1992) alignments (B) of 50, expectation (E) of 10, M=5, N=−4, and a comparison of both strands.

The BLAST algorithm also performs a statistical analysis of the similarity between two sequences (see, for example, Karlin, S. et al., “Applications and statistics for multiple high-scoring segments in molecular sequences,” Proc. Natl. Acad. Sci. U.S.A., Vol. 90, No. 12, pp. 5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, in some embodiments, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

### Antigen Binding Constructs

It is herein appreciated that the sequences within the CDR and/or FR can be of special relevance for various antigen binding constructs. In some embodiments, the value of the CDR and/or FR can be especially high, such as in a minibody or diabody, e.g., cys-diabody, arrangement.

Some aspects of the present disclosure relate to an antigen binding construct. In some embodiments, the antigen binding construct comprises: a variable light (V_{L}) domain comprising: a LCDR1 that is SEQ ID NO: 15; a LCDR2 that is any one of SEQ ID NO: 19, 21, or 23; a LCDR3 that is SEQ ID NO: 25; and/or a variable heavy (V_{H}) domain comprising: a HCDR1 that is SEQ ID NO: 27; a HCDR2 that is SEQ ID NO: 29; and a HCDR3 that is SEQ ID NO: 31. In some embodiments, the V_{L} comprises: a LCDR1 that is a LCDR1 in any one of SEQ ID NOs: 1-14, 90, and 91; a LCDR2 that is a LCDR2 in any one of SEQ ID NOs: 1-14, 90, and 91; a LCDR3 that is a LCDR 3 in any one of SEQ ID NOs: 1-14, 90, and 91; and the V_{H} comprises: a HCDR1 that is a HCDR1 in any one of SEQ ID NOs: 1-14, 87, and 88; a HCDR2 that is a HCDR 2 in any one of SEQ ID NOs: 1-14, 87, and 88; and a HCDR3 that is a HCDR3 in any one of SEQ ID NOs: 1-14, 87, and 88. In some embodiments, the antigen binding construct comprises: a variable heavy (V_{H}) domain comprising: a HCDR1 that is SEQ ID NO: 27; a HCDR2 that is SEQ ID NO: 29; and a HCDR3 that is SEQ ID NO: 31. In some embodiments, the antigen binding construct comprises: a variable heavy (V_{H}) domain comprising: a HCDR1 that is a HCDR1 in any one of SEQ ID NOs: 1-14, 87, and 88; a HCDR2 that is a HCDR 2 in any one of SEQ ID NOs: 1-14, 87, and 88; and a HCDR3 that is a HCDR3 in any one of SEQ ID NOs: 1-14, 87, and 88.

In some embodiments, the antigen binding construct comprises a minibody. In some embodiments, a minibody that binds to DLL3 is provided, wherein the minibody comprises: a single-chain variable fragment (scFv) that binds to DLL3. In some embodiments, the scFv comprises a variable light (V_{L}) domain linked to a variable heavy (V_{II}) domain. The V_{L} domain comprises: a LCDR1 that is SEQ ID NO: 15; a LCDR2 that is any one of SEQ ID NO: 19, 21, or 23; and a LCDR3 that is SEQ ID NO: 25. In some embodiments, the V_{H} domain comprises: a HCDR1 that is SEQ ID NO: 27; a HCDR2 that is SEQ ID NO: 29; a HCDR3 that is SEQ ID NO: 31. In some embodiments, the V_{L} comprises: a LCDR1 that is a LCDR1 in any one of SEQ ID NOs: 1-14, 90, and 91; a LCDR2 that is a LCDR2 in any one of SEQ ID NOs: 1-14, 90, and 91; a LCDR3 that is a LCDR 3 in any one of SEQ ID NOs: 1-14, 90, and 91; and the V_{H} comprises: a HCDR1 that is a HCDR1 in any one of SEQ ID NOs: 1-14, 87, and 88; a HCDR2 that is a HCDR 2 in any one of SEQ ID NOs: 1-14, 87, and 88; and a HCDR3 that is a HCDR3 in any one of SEQ ID NOs: 1-14, 87, and 88. In some embodiments, the minibody comprises: a variable heavy (V_{H}) domain comprising: a HCDR1 that is SEQ ID NO: 27; a HCDR2 that is SEQ ID NO: 29; and a HCDR3 that is SEQ ID NO: 31. In some embodiments, the minibody comprises: a variable heavy (V_{H}) domain comprising: a HCDR1 that is a HCDR1 in any one of SEQ ID NOs: 1-14, 87, and 88; a HCDR2 that is a HCDR 2 in any one of SEQ ID NOs: 1-14, 87, and 88; and a HCDR3 that is a HCDR3 in any one of SEQ ID NOs: 1-14, 87, and 88. The antigen binding construct further comprises a hinge-extension domain comprising a IgG hinge region, and a IgG C_{H}3 sequence.

In some embodiments, the antigen binding construct comprises a cys-diabody. In some embodiments, a cys-diabody that binds to DLL3 is provided, wherein the cys-diabody comprises a polypeptide that comprises: a single-chain variable fragment (scFv) comprising a variable light (V_{L}) domain linked to a variable heavy (V_{H}) domain. In some embodiments, the V_{L} domain comprises: a LCDR1 that is SEQ ID NO: 15; a LCDR2 that is any one of SEQ ID NO: 19, 21, or 23; and a LCDR3 that is SEQ ID NO: 25. In some embodiments, the V_{H} domain comprises: a HCDR1 that is SEQ ID NO: 27; a HCDR2 that is SEQ ID NO: 29; and a HCDR3 that is SEQ ID NO: 31. In some embodiments, the V_{L} comprises: a LCDR1 that is a LCDR1 in any one of SEQ ID NOs: 1-14, 90, and 91; a LCDR2 that is a LCDR2 in any one of SEQ ID NOs: 1-14, 90, and 91; a LCDR3 that is a LCDR 3 in any one of SEQ ID NOs: 1-14, 90, and 91; and the V_{H} comprises: a HCDR1 that is a HCDR1 in any one of SEQ ID NOs: 1-14, 87, and 88; a HCDR2 that is a HCDR 2 in any one of SEQ ID NOs: 1-14, 87, and 88; and a HCDR3 that is a HCDR3 in any one of SEQ ID NOs: 1-14, 87, and 88. In some embodiments, the V_{L} domain comprises: a LCDR1 that is SEQ ID NO: 15; a LCDR2 that is any one of SEQ ID NO: 19, 21, or 23; and a LCDR3 that is SEQ ID NO: 25, and the V_{H} domain comprises: a HCDR1 that is SEQ ID NO: 27; a HCDR2 that is SEQ ID NO: 29; and a HCDR3 that is SEQ ID NO: 31. In some embodiments, the cys-diabody comprises: a variable heavy (V_{H}) domain comprising: a HCDR1 that is SEQ ID NO: 27; a HCDR2 that is SEQ ID NO: 29; and a HCDR3 that is SEQ ID NO: 31. In some embodiments, the cys-diabody comprises: a variable heavy (V_{H}) domain comprising: a HCDR1 that is a HCDR1 in any one of SEQ ID NOs: 1-14, 87, and 88; a HCDR2 that is a HCDR 2 in any one of SEQ ID NOs: 1-14, 87, and 88; and a HCDR3 that is a HCDR3 in any one of SEQ ID NOs: 1-14, 87, and 88.

FIG. 1 shows some embodiments of a polypeptide sequence of antigen binding minibodies. (SEQ ID NO: 1-10).

In some embodiments, the antigen binding construct, minibody, or cys-diabody, comprise a CDR that is a CDR in Table 1.

TABLE 1

| | SEQUENCE |
|---|---|
| HCDR1 | KASGYTFTNYGMN |
| | (SEQ ID NO: 27) |
| HCDR2 | WINTYTGEPT |
| | (SEQ ID NO: 29) |
| HCDR3 | ARIGDSSPSDY |
| | (SEQ ID NO: 31) |
| LCDR1 | KASQSVSNDVVW |
| | (SEQ ID NO: 15) |
| LCDR2 | YASNRYTG |
| | (SEQ ID NO: 17) |
| LCDR2 | DASNRYTG |
| | (SEQ ID NO: 19) |
| LCDR2 | YASNRNTG |
| | (SEQ ID NO: 21) |
| LCDR2 | DASNRNTG |
| | (SEQ ID NO: 23) |
| LCDR3 | QQDYTSPWT |
| | (SEQ ID NO: 25) |

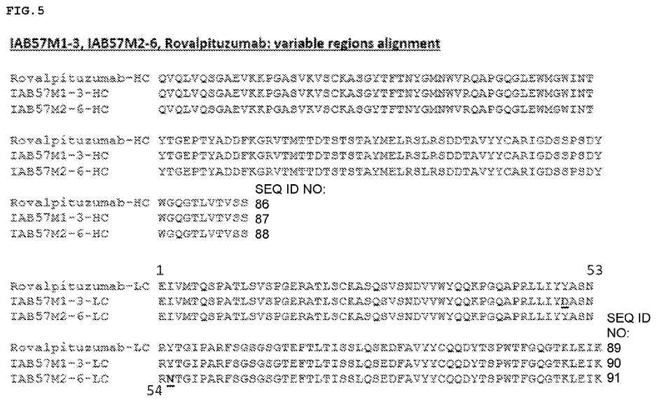

FIG. 4 shows some embodiments of an annotated polypeptide sequence of an antigen binding minibody.

FIG. 5 shows some embodiments of a variable region alignment of the polypeptide sequence of antigen binding minibodies and the Rovalpituzumab polypeptide sequence. In some embodiments, an antigen binding construct, minibody, or cys-diabody is provided that includes a sequence that is 60, 70, 80, 90, 95, 96, 97, 98, 99, or 100% identical to SEQ ID NO: 3, with residue number 50 of the V_{L} being a Y. In some embodiments, an antigen binding construct, minibody, or cys-diabody is provided that includes a sequence that is 60, 70, 80, 90, 95, 96, 97, 98, 99, or 100% identical to SEQ ID NO: 3, with residue number 55 of the V_{L} being a N.

FIG. 6 shows embodiments of a polypeptide sequence of human-DLL3. (SEQ ID NO: 50). The signal peptide is underlined, and a transmembrane domain is shown in bolded italics, in FIG. 6. In some embodiments, the antigen binding construct, minibody, and/or cys-diabody, binds to a protein having an amino acid sequence that is at least about 70%, 75%, 80%, 85%, 90%, 95%, 96%, 97%, 98% or 99% sequence identity to a DLL3 that is SEQ ID NO: 50 (with or without the signal peptide). In some embodiments, the antigen binding construct, minibody, and/or cys-diabody, binds to a protein having an amino acid sequence of SEQ ID NO:50, without the signal peptide.

In some embodiments, the V_{L} domain further comprises a LFR2 of SEQ ID NO: 35 or 37. In some embodiments, the V_{L} domain comprises an amino acid sequence having at least 90% identity to a V_{L} domain in SEQ ID NO: 48; and wherein the antigen binding construct, minibody, or cys-diabody comprises a Y50D and/or Y55N mutation, as numbered according to the numbering in SEQ ID NO: 1 (Kabat).

In some embodiments, the V_{L} domain further comprises a LFR2 of SEQ ID NO: 35 or 37. In some embodiments, the V_{L} domain comprises an amino acid sequence having at least 90% identity to a V_{L} domain in SEQ ID NO: 49; and wherein the antigen binding construct, minibody, or cys-diabody comprises a Y50D and/or Y55N mutation, as numbered according to the numbering in SEQ ID NO: 1 (Kabat).

In some embodiments, the V_{L} domain comprises an amino acid sequence having at least about 70%, 75%, 80%, 85%, 90%, 95%, 96%, 97%, 98% or 99% identity to a V_{L} domain in SEQ ID NO: 48, with residue 43 being an S, as numbered according to the numbering in SEQ ID NO: 1 (Kabat). In some embodiments, the V_{L} domain comprises an amino acid sequence having at least about 70%, 75%, 80%, 85%, 90%, 95%, 96%, 97%, 98% or 99% identity to a V_{L} domain in SEQ ID NO: 48 with residue 45 being an R, as numbered according to the numbering in SEQ ID NO: 1 (Kabat).

In some embodiments, the V_{L} domain comprises an amino acid sequence having at least about 70%, 75%, 80%, 85%, 90%, 95%, 96%, 97%, 98% or 99% identity to a V_{L} domain in SEQ ID NO: 49, with residue 43 being an S, as numbered according to the numbering in SEQ ID NO: 1 (Kabat). In some embodiments, the V_{L} domain comprises an amino acid sequence having at least about 70%, 75%, 80%, 85%, 90%, 95%, 96%, 97%, 98% or 99% identity to a V_{L} domain in SEQ ID NO: 49 with residue 45 being an R, as numbered according to the numbering in SEQ ID NO: 1 (Kabat).

FIG. 2 shows some embodiments of a polypeptide sequence of antigen binding cys-diabodies. (SEQ ID NO: 11-14). In some embodiments, the cys-diabody has at least about 70%, 75%, 80%, 85%, 90%, 95%, 96%, 97%, 98% or 99% sequence identity to a that is any of SEQ ID NO: 11-14. In some embodiments, the cys-diabody has at least about 70%, 75%, 80%, 85%, 90%, 95%, 96%, 97%, 98% or 99% sequence identity to a that is SEQ ID NO: 11. In some embodiments, the cys-diabody has at least about 70%, 75%, 80%, 85%, 90%, 95%, 96%, 97%, 98% or 99% sequence identity to a that is SEQ ID NO: 12. In some embodiments, the cys-diabody has at least about 70%, 75%, 80%, 85%, 90%, 95%, 96%, 97%, 98% or 99% sequence identity to a that is SEQ ID NO: 13. In some embodiments, the cys-diabody has at least about 70%, 75%, 80%, 85%, 90%, 95%, 96%, 97%, 98% or 99% sequence identity to a that is SEQ ID NO: 14.

In some embodiments, the antigen binding construct, minibody, or cys-diabody, comprises a FR that is a FR in Table 2. In some embodiments, the FR has at least about 70%, 75%, 80%, 85%, 90%, 95%, 96%, 97%, 98% or 99% sequence identity to a FR that is any of SEQ ID NO: 33-38. In some embodiments, the FR has at least about 70%, 75%, 80%, 85%, 90%, 95%, 96%, 97%, 98% or 99% sequence identity to a FR that is SEQ ID NO: 33. In some embodiments, the FR has at least about 70%, 75%, 80%, 85%, 90%, 95%, 96%, 97%, 98% or 99% sequence identity to a FR that is SEQ ID NO: 34. In some embodiments, the FR has at least about 70%, 75%, 80%, 85%, 90%, 95%, 96%, 97%, 98% or 99% sequence identity to a FR that is SEQ ID NO: 35. In some embodiments, the FR has at least about 70%, 75%, 80%, 85%, 90%, 95%, 96%, 97%, 98% or 99% sequence identity to a FR that is SEQ ID NO: 36. In some embodiments, the FR has at least about 70%, 75%, 80%, 85%, 90%, 95%, 96%, 97%, 98% or 99% sequence identity to a FR that is SEQ ID NO: 37. In some embodiments, the FR has at least about 70%, 75%, 80%, 85%, 90%, 95%, 96%, 97%, 98% or 99% sequence identity to a FR that is SEQ ID NO: 38.

TABLE 2

| | SEQUENCE |
|---|---|
| LFR2 | YQQKPGQAPRLLIY |
| | (SEQ ID NO: 33) |
| LFR2 | YQQKPGQAPOLLIY |
| | (SEQ ID NO: 35) |
| LFR2 | YQQKPGQSPOLLIY |
| | (SEQ ID NO: 37) |

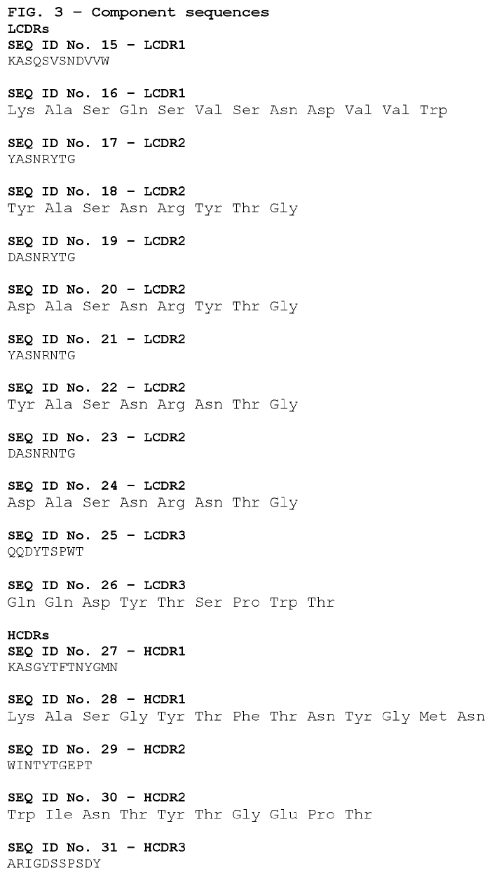

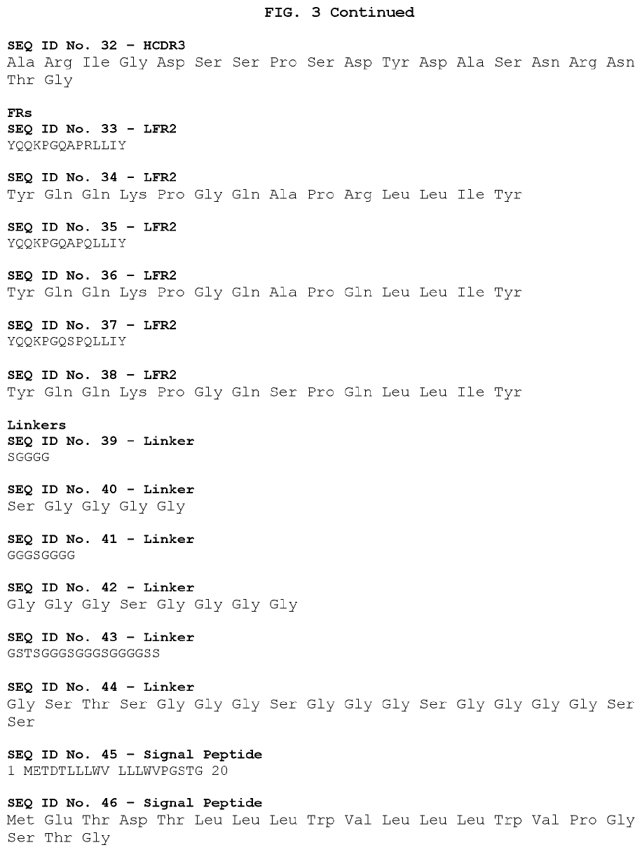

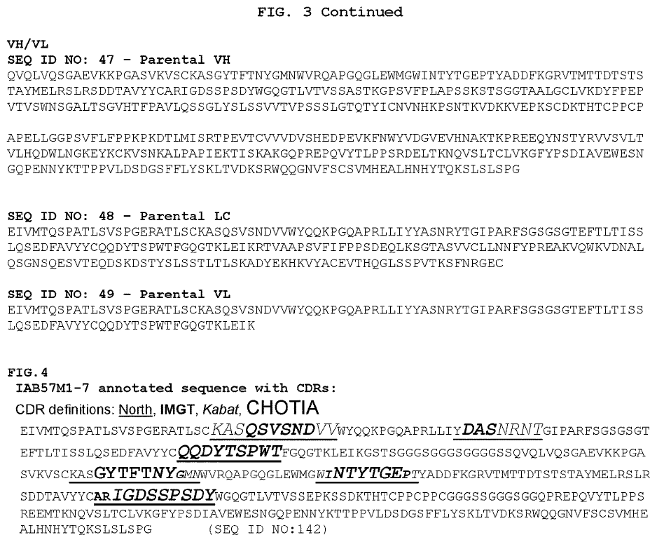

FIG. 3 shows some non-limiting embodiments of a polypeptide sequence of a LCDR (SEQ ID NO: 15-26), HCDR (SEQ ID NO: 27-32), LFR (SEQ ID NO: 33-38), linker (SEQ ID NO: 39-44), parental V_{H} (SEQ ID NO: 47), and parental V_{L} (SEQ ID NO: 48).

In some embodiments, the antigen binding construct, minibody, or cys-diabody has a disrupted cluster of surface-exposed, positively charged amino acids, and has enhanced biodistribution and/or pharmacokinetics compared to an original construct having an original cluster of surface-exposed, positively charged amino acids and from which the construct having the disrupted cluster is derived (e.g., solely by disrupting the original cluster in the original antibody, while other sequence-based features can otherwise remain the same). In some embodiments, the antigen binding construct, minibody, or cys-diabody has reduced kidney uptake when administered to a subject, compared to the original construct having the intact cluster of positively charged amino acids. In some embodiments, the antigen binding construct has a molecular weight of 15-160 kDa. In some embodiments, the antigen binding construct has a molecular weight of about 50-80 kDa. In some embodiments, the change in biodistribution due to disruption of the cluster is readily observed for an antigen binding construct having a molecular weight (as a dimer) of ~50-80 kDa, such as a minibody or a cys-diabody. In general, modification of the original antigen binding construct by disrupting the cluster of positively charged amino acids at least maintains the same binding specificity and binding affinity of the original antigen binding construct, while enhancing the biodistribution and/or pharmacokinetics.

In some embodiments, the antigen binding construct, minibody, or cys-diabody is a variant that includes at least one disrupted cluster of surface-exposed positively charged amino acids, the original cluster having at least two (e.g., 2, 3, 4, 5, 6 or more) surface-exposed, positively charged amino acids within about 30 angstroms of each other, where the variant varies from an original antigen binding construct that includes the original cluster by having a substitution of at least one of the surface-exposed, positively charged amino acids of the original cluster with one or more negatively charged or non-charged amino acid, whereby the original positive cluster is disrupted (compared to the original). In some embodiments, the original cluster has at least two (e.g., 2, 3, 4, 5, 6 or more) surface-exposed, positively charged amino acids within, or within about 30, 25, 20, 15, 10, 8, or 5 angstroms, or a distance in a range defined by any two of the preceding values (e.g., 5-30 angstroms, 5-25 angstroms, 5-15 angstroms, 10-20 angstroms, etc.) of each other. In some embodiments, the variant antigen binding construct exhibits reduced kidney uptake (e.g., when the variant antigen binding construct is radiolabeled and administered to a subject) when compared to the original antigen binding construct.

In some embodiments, the antigen binding construct, minibody, or cys-diabody includes at least one disrupted cluster of surface-exposed positively charged amino acids, the original cluster having at least two (e.g., 2, 3, 4, 5, 6 or more) surface-exposed, positively charged amino acids within about 30 angstroms of each other, where the variant varies from the original binding construct that includes the original cluster by having a substitution of at least one of the surface-exposed, positively charged amino acids of the original cluster with one or more negatively charged or non-charged amino acid, whereby the positive cluster is disrupted (compared to the binding construct). In some embodiments, the antigen binding construct, minibody, or cys-diabody includes a V_{L} having a variant framework region 2 (FR2) that has been modified from an original construct that includes the original sequence X₁X₂X₃X₄X₅X₆X₇ (SEQ ID NO: 92), where X₁ is a positively charged amino acid (e.g., a lysine or arginine), where X₂, X₃, X₅, and X₆ are each independently any negatively charged or a non-charged amino acid, and where X₄ and X₇ are each independently any amino acid with the proviso that at least one is a positively charged amino acid, where the sequence is outside of any CDR of the antigen binding construct, and where the at least one of X₄ and X₇ that is a positively charged amino acid is surface exposed and is part of the original cluster. In some embodiments, X₁ is part of the original cluster. In some embodiments, X₁ and the at least one of X₄ and X₇ that is a positively charged amino acid are part of the original cluster. In some embodiments, all of the positively charged amino acids of the original sequence are part of the original cluster. In some embodiments, X₂, X₃, X₅, and X₆ are each independently any non-charged amino acid. The antigen binding construct, e.g., variant antigen binding construct, can include the original sequence of X₁X₂X₃X₄X₅X₆X₇ (SEQ ID NO: 92) with the substitution of the at least one of the surface-exposed positively charged amino acids of the cluster with a negatively charged or non-charged amino acid that disrupts the original cluster. In some embodiments, the negatively charged or non-charged amino acid with which the at least one of the surface-exposed, positively charged amino acids of the original cluster is substituted is a glutamine (e.g., a K to Q, or R to Q substitution). In some embodiments, the framework region 2 (FR2) of the original antigen binding construct includes at least one of the following original sequences: KX₂X₃KX₅X₆K (SEQ ID NO: 143), where X₂, X₃, X₅, and X₆ are each independently any negatively charged or a non-charged amino acid; KX₂X₃X₄X₅X₆R (SEQ ID NO: 144), where X₂, X₃, X₄, X₅, and X₆ are each independently any negatively charged or a non-charged amino acid; or KX₂X₃X₄X₅X₆K (SEQ ID NO: 145), where X₂, X₃, X₄, X₅, and X₆ are each independently any negatively charged or a non-charged amino acid. In some embodiments, the framework region 2 (FR2) of the original antigen binding construct includes at least one of the following sequences: KPGKAPK (SEQ ID NO: 94), KPGQAPR (SEQ ID NO: 95), KPEKAPK (SEQ ID NO: 96), KPGKVPK (SEQ ID NO: 97), KPGQPPR (SEQ ID NO: 98), KPGQSPR (SEQ ID NO: 99), KPGLAPR (SEQ ID NO: 100), or KPGQPPK (SEQ ID NO: 101), corresponding to X₁X₂X₃X₄X₅X₆X₇ (SEQ ID NO: 92). In some embodiments, the original sequence is KPGKAPK (SEQ ID NO: 94) or KPGQAPR (SEQ ID NO: 95), corresponding to X₁X₂X₃X₄X₅X₆X₇ (SEQ ID NO: 92). In some embodiments, the antigen binding construct, minibody, or cys-diabody includes at least one of KPGQAPR (SEQ ID NO: 95), KPGQAPK (SEQ ID NO:146), KPGQAPQ (SEQ ID NO:111), KPGQSPQ (SEQ ID NO:110), and QQKPGQSPQ (SEQ ID NO: 147), e.g., in the V_{L} FR2.

In some embodiments, the antigen binding construct, minibody, or cys-diabody, further comprises a signal peptide that is a signal peptide in SEQ ID NO: 45.

In some embodiments, the antigen binding construct, minibody, or cys-diabody, comprise a linker (e.g., a peptide linker that links a V_{L} domain and a V_{H} domain of the antigen binding construct) that is any of SEQ ID NO: 39-44. In some embodiments, the antigen binding construct, minibody, or cys-diabody, comprise a linker that is a linker in Table 3.

TABLE 3

| | SEQUENCE |
|---|---|
| Linker | SGGGG |
| | (SEQ ID NO: 39) |
| Linker | GGGSGGGG |
| | (SEQ ID NO: 41) |
| Linker | GSTSGGGSGGGSGGGGSS |
| | (SEQ ID NO: 43) |

Table A shows some non-limiting embodiments of a hinge sequence of the antigen binding construct (e.g., minibody, scFv-Fc, Nanobody®-Fc) of the present disclosure. In some embodiments, the antigen binding construct (e.g., minibody, scFv-Fc, Nanobody®-Fc) includes a hinge region having any one of the sequences as set forth in Table A. In some embodiments, the antigen binding construct (e.g., minibody, scFv-Fc, Nanobody®-Fc) includes a hinge region having an upper hinge, core hinge, and a lower hinge including the sequence of any one of the upper hinge, core hinge, and a lower hinge, respectively, as set forth in Table A. In some embodiments, the antigen binding construct (e.g., minibody, scFv-Fc, Nanobody®-Fc) includes a hinge region having a sequence of any one of the full hinge sequences set forth in Table A. In some embodiments, the antigen binding construct includes an upper hinge having the sequence EPKSSDKTHT (SEQ ID NO: 102). In some embodiments, the antigen binding construct includes an upper hinge having the sequence EPGSSDGTHT (SEQ ID NO: 103). In some embodiments, the antigen binding construct includes a core hinge having the sequence CPPCPPC (SEQ ID NO: 104). In some embodiments, the antigen binding construct includes a core hinge having the sequence CPPCP (SEQ ID NO: 105). In some embodiments, the antigen binding construct includes a lower hinge having at least one of the following sequences: APELLGGP (SEQ ID NO: 106), GGGSSGGGSG (SEQ ID NO: 107), and APPVAGP (SEQ ID NO: 109).

TABLE A

| Full hinge | Upper hinge | Core hinge | Lower hinge |
|---|---|---|---|
| Human IgG1 NH1 | EPKSCDKTHT | CPPCP | APELLGGP |
| (SEQ ID NO: 123) | (SEQ ID NO: 112) | (SEQ ID NO: 105) | (SEQ ID NO: 106) |
| Human IgG1 EH1 | EPKSCDKTHT | CPPC | GGGSSGGGSG |
| (SEQ ID NO: 124) | (SEQ ID NO: 112) | (SEQ ID NO: 116) | (SEQ ID NO: 107) |
| Human IgG1 NH2 | EPKSSDKTHT | CPPCP | APELLGGP |
| (SEQ ID NO: 125) | (SEQ ID NO: 102) | (SEQ ID NO: 105) | (SEQ ID NO: 106) |
| Human IgG1 EH2 | EPKSSDKTHT | CPPC | GGGSSGGGSG |
| (SEQ ID NO: 126) | (SEQ ID NO: 102) | (SEQ ID NO: 116) | (SEQ ID NO: 107) |
| Human IgG1 NH3 | EPKSSDKTHT | CPPCPPC | APELLGGP |
| (SEQ ID NO: 127) | (SEQ ID NO: 102) | (SEQ ID NO: 104) | (SEQ ID NO: 106) |
| Human IgG1 EH3 | EPKSSDKTHT | CPPCPPC | GGGSSGGGSG |
| (SEQ ID NO: 128) | (SEQ ID NO: 102) | (SEQ ID NO: 104) | (SEQ ID NO: 107) |
| Human IgG1 NH4 | EPKSSDKTHT | CPPCVECPPC | APELLGGP |
| (SEQ ID NO: 129) | (SEQ ID NO: 102) | (SEQ ID NO: 117) | (SEQ ID NO: 106) |
| Human IgG1 EH4 | EPKSSDKTHT | CPPCVECPPC | GGGSSGGGSG |
| (SEQ ID NO: 130) | (SEQ ID NO: 102) | (SEQ ID NO: 117) | (SEQ ID NO: 107) |
| Human IgG1 NH5 | EPKSSDKTHT | CPPCPPCPPC | APELLGGP |
| (SEQ ID NO: 131) | (SEQ ID NO: 102) | (SEQ ID NO: 118) | (SEQ ID NO: 106) |
| Human IgG1 EH5 | EPKSSDKTHT | CPPCPPCPPC | GGGSSGGGSG |
| (SEQ ID NO: 132) | (SEQ ID NO: 102) | (SEQ ID NO: 118) | (SEQ ID NO: 107) |
| Human IgG2 NH1 | ERK | CCVECPPCP | APPVAGP |
| (SEQ ID NO: 133) | (SEQ ID NO: 113) | (SEQ ID NO: 119) | (SEQ ID NO: 108) |
| Human IgG2 EH1 | ERK | CCVECPPCP | GGGSSGGGSG |
| (SEQ ID NO: 134) | (SEQ ID NO: 113) | (SEQ ID NO: 119) | (SEQ ID NO: 107) |
| Human IgG2 NH2 | ERK | SCVECPPCP | APPVAGP |
| (SEQ ID NO: 135) | (SEQ ID NO: 113) | (SEQ ID NO: 120) | (SEQ ID NO: 108) |
| Human IgG2 EH2 | ERK | SCVECPPCP | GGGSSGGGSG |
| (SEQ ID NO: 136) | (SEQ ID NO: 113) | (SEQ ID NO: 120) | (SEQ ID NO: 107) |
| IgG3/IgG1 EH6 (SEQ | ELKTPLGDTTHT | CVECPPC | GGGSSGGGSG |
| ID NO: 137) | (SEQ ID NO: 114) | (SEQ ID NO: 121) | (SEQ ID NO: 107) |
| IgG3/IgG1 EH7 (SEQ | ELKTPLGDTTHT | CPPCPPC | GGGSSGGGSG |
| ID NO: 138) | (SEQ ID NO: 114) | (SEQ ID NO: 104) | (SEQ ID NO: 107) |
| IgG3/IgG1 EH8 (SEQ | ELKTPLGDTTHT | CPPCPPCPPC | GGGSSGGGSG |
| ID NO: 139) | (SEQ ID NO: 114) | (SEQ ID NO: 122) | (SEQ ID NO: 107) |
| IgG4 NH (SEQ ID | ESKYGPP | CPPCP | APELLGGP |
| NO: 140) | (SEQ ID NO: 115) | (SEQ ID NO: 105) | (SEQ ID NO: 106) |
| IgG4 EH (SEQ ID | ESKYGPP | CPPCP | GGGSSGGGSG |
| NO: 141) | (SEQ ID NO: 115) | (SEQ ID NO: 105) | (SEQ ID NO: 107) |

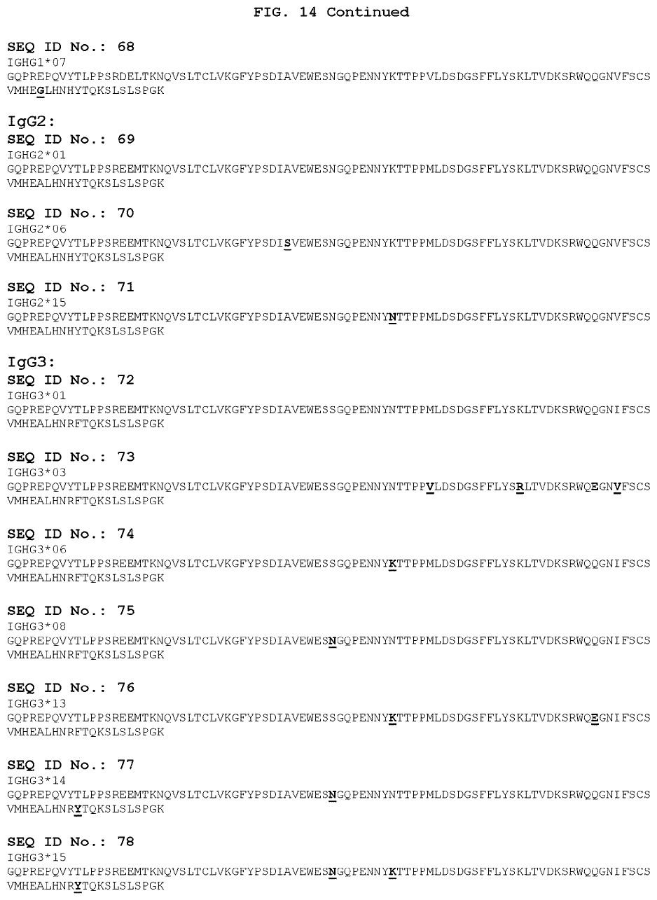

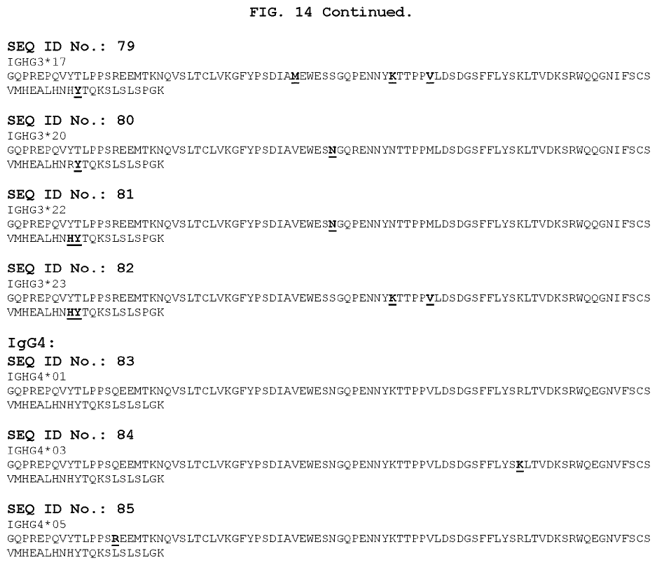

FIG. 14 shows some non-limiting embodiments of a C_{H}3 polypeptide sequence (SEQ ID NO: 65-85).

In some embodiments, the antigen binding construct, minibody, or cys-diabody, comprises a C_{H}3 domain that is a C_{H}3 domain in Table 4. In some embodiments, the C_{H}3 domain is an IgG C_{H}3 domain. In some embodiments, the IgG C_{H}3 domain is an IgG1, IgG2, IgG3, and/or IgG 4 C_{H}3 domain. In some embodiments, the C_{H}3 domain comprises a germline C_{H}3 domain. In some embodiments, the C_{H}3 domain comprises one or more allotypes. In some embodiments, the IgG1 C_{H}3 domain is any of SEQ ID Nos. 65-68. In some embodiments, the C_{H}3 domain has at least about 70%, 75%, 80%, 85%, 90%, 95%, 96%, 97%, 98% or 99% sequence identity to a C_{H}3 domain that is any of SEQ ID NO: 65-68. In some embodiments, the C_{H}3 domain has at least about 70%, 75%, 80%, 85%, 90%, 95%, 96%, 97%, 98% or 99% sequence identity to a C_{H}3 domain that is SEQ ID NO: 65. In some embodiments, the hinge region has at least about 70%, 75%, 80%, 85%, 90%, 95%, 96%, 97%, 98% or 99% sequence identity to a C_{H}3 domain that is SEQ ID NO: 66. In some embodiments, the C_{H}3 domain has at least about 70%, 75%, 80%, 85%, 90%, 95%, 96%, 97%, 98% or 99% sequence identity to a C_{H}3 domain that is SEQ ID NO: 67. In some embodiments, the C_{H}3 domain has at least about 70%, 75%, 80%, 85%, 90%, 95%, 96%, 97%, 98% or 99% sequence identity to a C_{H}3 domain that is SEQ ID NO: 68. In some embodiments, the C_{H}3 domain is an IgG2 C_{H}3 domain. In some embodiments, the IgG2 C_{H}3 domain is any of SEQ ID Nos. 69-71. In some embodiments, the C_{H}3 domain has at least about 70%, 75%, 80%, 85%, 90%, 95%, 96%, 97%, 98% or 99% sequence identity to a C_{H}3 domain that is any of SEQ ID NO: 69-71. In some embodiments, the C_{H}3 domain has at least about 70%, 75%, 80%, 85%, 90%, 95%, 96%, 97%, 98% or 99% sequence identity to a C_{H}3 domain that is SEQ ID NO: 69. In some embodiments, the C_{H}3 domain has at least about 70%, 75%, 80%, 85%, 90%, 95%, 96%, 97%, 98% or 99% sequence identity to a C_{H}3 domain that is SEQ ID NO: 70. In some embodiments, the C_{H}3 domain has at least about 70%, 75%, 80%, 85%, 90%, 95%, 96%, 97%, 98% or 99% sequence identity to a C_{H}3 domain that is SEQ ID NO: 71. In some embodiments, the hinge region is an IgG3 C_{H}3 domain. In some embodiments, the IgG3 C_{H}3 domain is any of SEQ ID Nos. 72-82. In some embodiments, the C_{H}3 domain has at least about 70%, 75%, 80%, 85%, 90%, 95%, 96%, 97%, 98% or 99% sequence identity to a C_{H}3 domain that is any of SEQ ID NO: 72-82. In some embodiments, the C_{H}3 domain has at least about 70%, 75%, 80%, 85%, 90%, 95%, 96%, 97%, 98% or 99% sequence identity to a C_{H}3 domain that is SEQ ID NO: 72. In some embodiments, the C_{H}3 domain has at least about 70%, 75%, 80%, 85%, 90%, 95%, 96%, 97%, 98% or 99% sequence identity to a C_{H}3 domain that is SEQ ID NO: 73. In some embodiments, the C_{H}3 domain has at least about 70%, 75%, 80%, 85%, 90%, 95%, 96%, 97%, 98% or 99% sequence identity to a C_{H}3 domain that is SEQ ID NO: 74. In some embodiments, the C_{H}3 domain has at least about 70%, 75%, 80%, 85%, 90%, 95%, 96%, 97%, 98% or 99% sequence identity to a C_{H}3 domain that is SEQ ID NO: 75. In some embodiments, the C_{H}3 domain has at least about 70%, 75%, 80%, 85%, 90%, 95%, 96%, 97%, 98% or 99% sequence identity to a C_{H}3 domain that is SEQ ID NO: 76. In some embodiments, the C_{H}3 domain has at least about 70%, 75%, 80%, 85%, 90%, 95%, 96%, 97%, 98% or 99% sequence identity to a C_{H}3 domain that is SEQ ID NO: 77. In some embodiments, the C_{H}3 domain has at least about 70%, 75%, 80%, 85%, 90%, 95%, 96%, 97%, 98% or 99% sequence identity to a C_{H}3 domain that is SEQ ID NO: 78. In some embodiments, the C_{H}3 domain has at least about 70%, 75%, 80%, 85%, 90%, 95%, 96%, 97%, 98% or 99% sequence identity to a C_{H}3 domain that is SEQ ID NO: 79. In some embodiments, the C_{H}3 domain has at least about 70%, 75%, 80%, 85%, 90%, 95%, 96%, 97%, 98% or 99% sequence identity to a C_{H}3 domain that is SEQ ID NO: 80. In some embodiments, the C_{H}3 domain has at least about 70%, 75%, 80%, 85%, 90%, 95%, 96%, 97%, 98% or 99% sequence identity to a C_{H}3 domain that is SEQ ID NO: 81. In some embodiments, the C_{H}3 domain has at least about 70%, 75%, 80%, 85%, 90%, 95%, 96%, 97%, 98% or 99% sequence identity to a C_{H}3 domain that is SEQ ID NO: 82. In some embodiments, the C_{H}3 domain is an IgG2 C_{H}3 domain. In some embodiments, the IgG2 C_{H}3 domain is any of SEQ ID Nos. 83-85. In some embodiments, the C_{H}3 domain has at least about 70%, 75%, 80%, 85%, 90%, 95%, 96%, 97%, 98% or 99% sequence identity to a C_{H}3 domain that is any of SEQ ID NO: 83-85. In some embodiments, the C_{H}3 domain has at least about 70%, 75%, 80%, 85%, 90%, 95%, 96%, 97%, 98% or 99% sequence identity to a C_{H}3 domain that is SEQ ID NO: 83. In some embodiments, the C_{H}3 domain has at least about 70%, 75%, 80%, 85%, 90%, 95%, 96%, 97%, 98% or 99% sequence identity to a C_{H}3 domain that is SEQ ID NO: 84. In some embodiments, the C_{H}3 domain has at least about 70%, 75%, 80%, 85%, 90%, 95%, 96%, 97%, 98% or 99% sequence identity to a C_{H}3 domain that is SEQ ID NO: 85.

TABLE 4

| IgG CH3 Domain | SEQ ID No. |
|---|---|
| IgG1 | |
| GQPREPQVYTLPPSRDELTKNQVSLTCLVKGFYPSDIAVEWESNGQPENNYKTTPPV | SEQ ID NO: 65 |
| LDSDGSFFLYSKLTVDKSRWQQGNVFSCSVMHEALHNHYTQKSLSLSPGK | |
| GQPREPQVYTLPPSREEMTKNQVSLTCLVKGFYPSDIAVEWESNGQPENNYKTTPPV | SEQ ID NO: 66 |
| LDSDGSFFLYSKLTVDKSRWQQGNVFSCSVMHEALHNHYTQKSLSLSPGK | |
| GQPREPQVYTLPPSRDELTKNQVSLTCLVKGFYPSDIAVEWESNGQPENNYKTTPPV | SEQ ID NO: 67 |
| LDSDGSFFLYSKLTVDKSRWQQGNIFSCSVMHEALHNHYTQKSLSLSPGK | |
| GQPREPQVYTLPPSRDELTKNQVSLTCLVKGFYPSDIAVEWESNGQPENNYKTTPPV | SEQ ID NO: 68 |
| LDSDGSFFLYSKLTVDKSRWQQGNVFSCSVMHEGLHNHYTQKSLSLSPGK | |
| IgG2 | |
| GQPREPQVYTLPPSREEMTKNQVSLTCLVKGFYPSDIAVEWESNGQPENNYKTTPPM | SEQ ID NO: 69 |
| LDSDGSFFLYSKLTVDKSRWQQGNVFSCSVMHEALHNHYTQKSLSLSPGK | |
| GQPREPQVYTLPPSREEMTKNQVSLTCLVKGFYPSDISVEWESNGQPENNYKTTPPM | SEQ ID NO: 70 |
| LDSDGSFFLYSKLTVDKSRWQQGNVFSCSVMHEALHNHYTQKSLSLSPGK | |
| GQPREPQVYTLPPSREEMTKNQVSLTCLVKGFYPSDIAVEWESNGQPENNYNTTPPM | SEQ ID NO: 71 |
| LDSDGSFFLYSKLTVDKSRWQQGNVFSCSVMHEALHNHYTQKSLSLSPGK | |
| IgG3 | |
| GQPREPQVYTLPPSREEMTKNQVSLTCLVKGFYPSDIAVEWESSGQPENNYNTTPPM | SEQ ID NO: 72 |
| LDSDGSFFLYSKLTVDKSRWQQGNIFSCSVMHEALHNRFTQKSLSLSPGK | |
| CQPREPQVYTLPPSREEMTKNQVSLTCLVKGFYPSDIAVEWESSCQPENNYNTTPPV | SEQ ID NO: 73 |
| LDSDGSFFLYSRLTVDKSRWQEGNVFSCSVMHEALHNRFTQKSLSLSPGK | |
| GQPREPQVYTLPPSREEMTKNQVSLTCLVKGFYPSDIAVEWESSGQPENNYKTTPPM | SEQ ID NO: 74 |
| LDSDGSFFLYSKLTVDKSRWQQGNIFSCSVMHEALHNRFTQKSLSLSPGK | |
| GQPREPQVYTLPPSREEMTKNQVSLTCLVKGFYPSDIAVEWESNGQPENNYNTTPPM | SEQ ID NO: 75 |
| LDSDGSFFLYSKLTVDKSRWQQGNIFSCSVMHEALHNRFTQKSLSLSPGK | |
| GQPREPQVYTLPPSREEMTKNQVSLTCLVKGFYPSDIAVEWESSGQPENNYKTTPPM | SEQ ID NO: 76 |
| LDSDGSFPLYSKLTVDKSRWQEGNIESCSVMHEALHNRETQKSLSLSPGK | |
| GQPREPQVYTLPPSREEMTKNQVSLTCLVKGFYPSDIAVEWESNGQPENNYNTTPPM | SEQ ID NO: 77 |
| LDSDGSFFLYSKLTVDKSRWQQGNIFSCSVMHEALHNRYTQKSLSLSPGK | |
| GQPREPQVYTLPPSREEMTKNQVSLTCLVKGFYPSDIAVEWESNGQPENNYKTTPPM | SEQ ID NO: 78 |
| LDSDGSFFLYSKLTVDKSRWQQGNIFSCSVMHEALHNRYTQKSLSLSPGK | |
| GQPREPQVYTLPPSREEMTKNQVSLTCLVKGFYPSDIAMEWESSGQPENNYKTTPPV | SEQ ID NO: 79 |
| LDSDGSFFLYSKLTVDKSRWQQGNIFSCSVMHEALHNHYTQKSLSLSPGK | |
| GQPREPQVYTLPPSREEMTKNQVSLTCLVKGFYPSDIAVEWESNGQRENNYNTTPPM | SEQ ID NO: 80 |
| LDSDGSFFLYSKLTVDKSRWQQGNIFSCSVMHEALHNRYTQKSLSLSPGK | |
| GQPREPQVYTLPPSREEMTKNQVSLTCLVKGFYPSDIAVEWESNGQPENNYNTTPPM | SEQ ID NO: 81 |
| LDSDGSFFLYSKLTVDKSRWQQGNIFSCSVMHEALHNHYTQKSLSLSPGK | |
| GQPREPQVYTLPPSREEMTKNQVSLTCLVKGFYPSDIAVEWESSGQPENNYKTTPPV | SEQ ID NO: 82 |
| LDSDGSFFLYSKLTVDKSRWQQGNIFSCSVMHEALHNHYTQKSLSLSPGK | |
| IgG4 | |
| GQPREPQVYTLPPSQEEMTKNQVSLTCLVKGFYPSDIAVEWESNGQPENNYKTTPPV | SEQ ID NO: 83 |
| LDSDGSFFLYSRLTVDKSRWQEGNVFSCSVMHEALHNHYTQKSLSLSLGK | |
| GQPREPQVYTLPPSQEEMTKNQVSLTCLVKGFYPSDIAVEWESNGQPENNYKTTPPV | SEQ ID NO: 84 |
| LDSDGSFFLYSKLTVDKSRWQEGNVFSCSVMHEALHNHYTQKSLSLSLGK | |
| GQPREPQVYTLPPSREEMTKNQVSLTCLVKGFYPSDIAVEWESNGQPENNYKTTPPV | SEQ ID NO: 85 |
| LDSDGSFFLYSRLTVDKSRWQEGNVFSCSVMHEALHNHYTQKSLSLSLGK | |

In some embodiments, the antigen binding construct includes a Fe region (e.g., a scFv-Fc, a Nanobody®-Fc). In some embodiments, the antigen binding construct includes an amino acid sequence identical to the amino acid sequence of a native or naturally-occurring Fc region (e.g., a human IgG1 Fc region). In some embodiments, the Fc region includes an amino acid sequence of SEQ DID NO: 109, as set forth below.

```
(SEQ ID NO: 109)
              230 240 250 260 270 280 290
              | | | | | | |
THTCPPCPAPELLGGPSVFLFPPKPKDTLMISRTPEVTCVVVDVSHEDPEVKFNWYVDGVEVHNAKTK
                  300 310 320 330 340 350 360
                  | | | | | | |
PREEQYNSTYRVVSVLTVLHQDWLNGKEYKCKVSNKALPAPIEKTISKAKGQPREPQVYTLPPSREEMTK
                    370 380 390 400 410 420 430
                  | | | | | | |
NQVSLTCLVKGFYPSDIAVEWESNGQPENNYKTTPPVLDSDGSFFLYSKLTVDKSRWQQGNVFSCSVMHE
                  440
                  |
ALHNHYTQKSLSLSPGK
```

In some embodiments, the Fc region includes an amino acid sequence at least 70, 80, 90, 95, 96, 97, 98, 99, or about 100% identical or identical by a percentage in a range defined by any two of the preceding values (e.g., 80-100%, 85-97%, 85-95%, 90-100%, etc.), to SEQ ID NO: 109. In some embodiments, the Fc region includes one or more (e.g., 1, 2, 3, 4, 5, 6, 7, 8, 9, 10 or more) mutations (e.g., substitutions) that changes (e.g., increase or reduce) an effector function (e.g., FcγR binding) and/or binding to the Fe neonatal receptor (FcRn). In some embodiments, the Fc region includes an amino acid sequence of SEQ ID NO: 109, with one or more (e.g., 1, 2, 3, 4, 5, 6, 7, 8, 9, 10 or more) mutations (e.g., substitutions) that changes (e.g., increase or reduce) an effector function (e.g., FcγR binding) and/or binding to the Fc neonatal receptor (FcRn). In some embodiments, the Fc region includes one or more (e.g., 1, 2, 3, 4, 5, 6, 7, 8, 9, 10 or more) mutations (e.g., substitutions) that reduces an effector function (e.g., FcγR binding) and/or binding to the Fc neonatal receptor (FcRn). In some embodiments, the Fc region includes an amino acid sequence of SEQ ID NO: 109, with one or more (e.g., 1, 2, 3, 4, 5, 6, 7, 8, 9, 10 or more) mutations (e.g., substitutions) that reduces an effector function (e.g., FcγR binding) and/or binding to the Fc neonatal receptor (FcRn). In some embodiments, the one or more mutations that reduces FcRn binding is a mutation (e.g., substitution) of any one or more (e.g., one, two or all three) of I253, H310, and H435 (EU numbering). In some embodiments, the one or more mutations that reduces binding to the FcRn is any one or more (e.g., one, two or all three) of I253A, H310A, and H435A (EU numbering). In some embodiments, the Fc region includes one or more (e.g., one, two or all three) of I253A, H310A, and H435A (EU numbering). In some embodiments, the one or more mutations that reduces binding to the FcγR is a mutation (e.g., substitution) of N297 (EU numbering). In some embodiments, the one or more mutations that reduces binding to the Fc neonatal receptor is N297Q, N297A or N297G (EU numbering). In some embodiments, the Fc region includes N297Q (EU numbering). In some embodiments, the one or more mutations that reduces Fc effector function is a mutation (e.g., substitution) of any one or more of: L234, L235, G236, G237, P238, H268, K322, L328, P329, A330, P331. In some embodiments, the one or more mutations that reduces Fe effector function is any one or more of: L234A, L235A, G236R, G237A, P238S, H268A, K322A, L328R, P329G, A330S, P331S. Further non-limiting examples of known mutations that change Fc effector function may be found in Wilkinson and Hale (2022) Systematic analysis of the varied designs of 819 therapeutic antibodies and Fc fusion proteins assigned international nonproprietary names. MAbs. 2022 January-December; 14(1):2123299. doi: 10.1080/19420862.2022.2123299. In some embodiments, the Fc region does not include a C-terminal lysine.

In some embodiments, the antigen binding construct is an scFv-Fc. In some embodiments, the scFv-Fc includes any one of the scFv that binds to DLL3, e.g., having a variable light (V_{L}) domain linked to the variable heavy (V_{H}) domain as described herein, a hinge domain, and an Fc region. In some embodiments, the scFv-Fc includes any one of the scFv that binds to DLL3, e.g., having a variable light (V_{L}) domain linked to the variable heavy (V_{H}) domain as described herein, linked to an Fc region via a hinge domain, as described herein. In some embodiments, the hinge domain is any suitable hinge domain as described herein (e.g., Table A). In some embodiments, the antigen binding construct is a Nanobody®-Fc (or a single domain fragment fused to an Fc region). In some embodiments, the Nanobody®-Fc includes a variable domain (VHH) having any one or more of the HCDR1, HCDR2 and HCDR3 sequences, as described herein. In some embodiments, the Nanobody®-Fc includes a variable domain (VHH) having a HCDR1 that is SEQ ID NO: 27; a HCDR2 that is SEQ ID NO: 29; and a HCDR3 that is SEQ ID NO: 31. In some embodiments, the Nanobody®-Fc includes a variable domain (VHH) having a HCDR1 that is a HCDR1 in any one of SEQ ID NOs: 1-14, 87, and 88; a HCDR2 that is a HCDR 2 in any one of SEQ ID NOs: 1-14, 87, and 88; and a HCDR3 that is a HCDR3 in any one of SEQ ID NOs: 1-14, 87, and 88. In some embodiments, the Nanobody®-Fc includes a V_{H} domain having any one or more (e.g., any 1, 2 or all 3) of the HCDR1, HCDR2 and HCDR3 sequences as described herein linked to a Fc region via a hinge domain, as described herein.

In some embodiments, the antigen binding construct includes at least one modification. Exemplary modifications include, but are not limited to, antigen binding constructs that have been modified by glycosylation, acetylation, pegylation, phosphorylation, amidation, derivatization by known protecting/blocking groups, conjugation to metal chelator, fluorescence or infrared dye, or conjugation to a toxin, proteolytic cleavage, and linkage to a cellular ligand or other protein. Any of numerous chemical modifications may be carried out by known techniques, including, but not limited to, amine coupling to amine containing amino acids, cystine coupling, specific chemical cleavage, acetylation, and metabolic synthesis of tunicamycin. In some embodiments, the derivative can contain one or more non-natural amino acids.

In some embodiments, the antigen binding construct is conjugated to another substance to form an anti-target conjugate. The conjugates described herein can be prepared by known methods of linking antigen binding constructs with lipids, carbohydrates, protein or other atoms and molecules. In some embodiments, the conjugate is formed by site-specific conjugation using a suitable linkage or bond. Site-specific conjugation is more likely to preserve the binding activity of an antigen binding construct. The substance may be conjugate or attached at the hinge region of a reduced antigen binding construct via thioether bond formation. In some embodiments, tyrosine conjugation can be employed. Other linkages or bonds used to form the conjugate can include, but are not limited to, a covalent bond, a non-covalent bond, a disulfide linkage, a hydrazone linkage, an ester linkage, an amido linkage, and amino linkage, an imino linkage, a thiosemicarbazone linkage, a semicarbazone linkage, an oxime linkage and a carbon-carbon linkage. In some embodiments, no cysteine or other linking aspect, need be included in the antigen binding construct.

In some embodiments, the antigen binding construct, minibody, and/or cys-diabody are conjugated to a chemotherapeutic agent. Chemotherapeutic agents are often cytotoxic or cytostatic in nature and may include alkylating agents, antimetabolites, anti-tumor antibiotics, topoisomerase inhibitors, mitotic inhibitors hormone therapy, targeted therapeutics and immunotherapeutics. In some embodiments the chemotherapeutic agents that may be used as detectable markers in accordance with the embodiments of the disclosure are selected from among those chemotherapeutic agents defined elsewhere herein.

In some embodiments, the antigen binding construct, minibody, and/or cys-diabody are conjugated to a toxin. Toxins that may be used in accordance with the embodiments of the disclosure include, but are not limited to, Auristatin E, Auristatin F, Dolastatin 10, Dolastatin 15, combretastatin and their analogs, maytansinoid, calicheamicin, alpha-amanitin, pyrrolobenzodiazepine dimers, epothilones, duocarmycin and their analogs, tubulysin D, basillistatins, ricin, abrin, ribonuclease (RNase), DNase I, Staphylococcal enterotoxin-A, pokeweed antiviral protein, gelonin, diphtheria toxin, Pseudomonas exotoxin, and Pseudomonas endotoxin.

In some embodiments, the antigen binding construct, minibody, and/or cys-diabody are conjugated to a radiolabel. In some embodiments, the radiolabel comprises Iodine-131, a beta-emitter or alpha-emitter, such as, Yttrium-90, Copper-67, Terbium-149, Terbium-161, Lutetium-177, Astatine-211, Lead-212, Bismuth-212, Actinium-225, Bismuth-213, or 227-Thorium, a positron emitter such as Zirconium-89, Copper-64, Gallium-68, Fluorine-18, Indium-111, Iodine-124. In some embodiments, the radiolabel comprises Terbium-149, Terbium-161, or Lead-212. In some embodiments, the antigen binding construct, minibody, and/or cys-diabody, are conjugated to a gamma emitter. In some embodiments the radio label comprises a gamma emitter.

In some embodiments, the antigen binding construct, minibody, and/or cys-diabody are conjugated to a radioisotope, a fluorescent compound, a bioluminescent compound, chemiluminescent compound, a metal chelator or an enzyme. In some embodiments, the at least one payload is comprises ¹⁸F, ⁸F-FAC, ³²P, ³³P, ⁴⁵Ti, ⁴⁷Sc, ⁵²Fe, S⁹Fe, ⁶²Cu, ⁶⁴Cu, ⁶⁷Cu, ⁶⁷Ga, ⁶⁸Ga, ⁷⁵Sc, ⁷⁷As, ⁸⁶Y, ⁹⁰Y, ⁸⁹Sr, ⁸⁹Zr, ⁹⁴Tc, ⁹⁴Tc, ⁹⁹mTc, ⁹⁹Mo, ¹⁰⁵Pd, ¹⁰⁵R, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁴⁹Tb, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁷Th and ²²⁵Ac, or any combination thereof. In some embodiments, the at least one payload is comprises ¹⁴⁹Tb, ¹⁶¹Tb, or ²¹²Pb.

In some embodiments, the antigen binding construct is an antibody (~140-170 kDa), a multi-specific antibody (~100 kDa and greater), a single-arm or one-arm antibody (~110 kDa and less), a modified antibody, or the like. In some embodiments, the antigen binding construct, minibody, or cys-diabody, binds specifically to DLL3. In some embodiments, the antigen binding construct, minibody, or cys-diabody, is bispecific. In some embodiments, the antigen binding construct, minibody, or cys-diabody, is bivalent. In some embodiments, the antigen binding construct, minibody, or cys-diabody comprises a monovalent scFv. In some embodiments, the antigen binding construct is a scFv-Fc (e.g., a scFv fused to a Fe). In some embodiments, the antigen binding construct is a Nanobody®-Fc (e.g., a Nanobody® (or single domain fragment) fused to a Fe).

In some embodiments, the order of the variable domains, from N terminus to C terminus of the antigen binding construct, minibody, or cys-diabody, from N terminus to C terminus of the polypeptide is V_{L}, V_{H}. In some embodiments, the order of the variable domains, from N terminus to C terminus of the polypeptide is V_{H}, V_{L}.

In some embodiments, the antigen binding construct, minibody, or cys-diabody is a humanized antigen binding construct, minibody, or cys-diabody. In some embodiments, the antigen binding constructs, minibodies, and/or cys-diabodies of any of FIG. 1-6 can be reformatted from an antigen binding construct, minibody, or cys-diabody, to an antigen binding construct, minibody, and/or cys-diabody. For example, the minibodies presented in FIG. 1 may be reformatted to antigen binding constructs or cys-diabodies. The cys-diabodies presented in FIG. 2 may be reformatted to antigen binding constructs or minibodies. For example, the minibodies or cys-diabodies presented in FIGS. 1 and 2 can be reformatted to scFv-Fc.

In some embodiments, a minibody that binds to DLL3 includes: a single-chain variable fragment (scFv) that binds to DLL3, the scFv comprising a variable light (V_{L}) domain linked to a variable heavy (V_{H}) domain, the V_{L} domain comprising: a LCDR1 that is SEQ ID NO: 15; a LCDR2 that is any one of SEQ ID NO: 19, 21, or 23; a LCDR3 that is SEQ ID NO: 25 and the V_{H} domain comprising: a HCDR1 that is SEQ ID NO: 27; a HCDR2 that is SEQ ID NO: 29; a HCDR3 that is SEQ ID NO: 31; a hinge region; a IgG C_{H}3 sequence; and a therapeutic agent, toxic payload, and/or a detectable marker.

In some embodiments, a cys-diabody that binds to DLL3 includes: a polypeptide that comprises: a single-chain variable fragment (scFv) comprising a variable light (V_{L}) domain linked to a variable heavy (V_{H}) domain; the V_{L} domain comprising: a LCDR1 of LCDR1 of SEQ ID NO: 15; a LCDR2 of a LCDR2 of SEQ ID NO: 19, 21, or 23; a LCDR3 of a LCDR3 of SEQ ID NO: 25; a V_{H} domain comprising: a HCDR1 of a HCDR1 of SEQ ID NO: 27; a HCDR2 of a HCDR2 of SEQ ID NO: 29; a HCDR3 of a HCDR3 of SEQ ID NO: 31; an extension sequence; and a therapeutic agent, toxic payload, and/or a detectable marker.

In some embodiments, a scFv-Fc that binds to DLL3 includes: a single-chain variable fragment (scFv) that binds to DLL3, the scFv comprising a variable light (V_{L}) domain linked to a variable heavy (V_{H}) domain, the V_{L} domain comprising: a LCDR1 that is SEQ ID NO: 15; a LCDR2 that is any one of SEQ ID NO: 19, 21, or 23; a LCDR3 that is SEQ ID NO: 25 and the V_{H} domain comprising: a HCDR1 that is SEQ ID NO: 27; a HCDR2 that is SEQ ID NO: 29; a HCDR3 that is SEQ ID NO: 31; a hinge region; a Fc region; and a therapeutic agent, toxic payload, and/or a detectable marker.

In some embodiments, the humanized antigen binding construct, minibody, or cys-diabody comprises: a LCDR1 of LCDR1 of SEQ ID NO: 15; a LCDR2 of LCDR2 of any one of SEQ ID NO: 19, 21, or 23; a LCDR3 of LCDR3 of SEQ ID NO: 25; a HCDR1 of a HCDR1 of SEQ ID NO: 27; a HCDR2 of HCDR2 of SEQ ID NO: 29; and a HCDR3 of HCDR3 of SEQ ID NO: 31.

In some embodiments, the V_{L} domain comprises, or further comprises a LFR2 of a LFR2 of SEQ ID NO: 35 or 37.

### Properties

FIG. 7 is a bar graph showing embodiments of minibody expression yield in Expi293 mammalian cells.

In some embodiments, expression yield of the antigen binding construct, minibody, and/or cys-diabody is determined using ultraviolet-visible spectroscopy (UV-Vis or UV/Vis). In some embodiments, expression yield of the antigen binding construct, minibody, and/or cys-diabody is determined using chromatographic techniques. In some embodiments, expression of the antigen binding construct, minibody, and/or cys-diabody is determined using biolayer interferometry. In some embodiments, the expression yield of the antigen binding construct, minibody, or cys-diabody is, is about, or is at least 1, 5, 10, 15, 20, 25, 30, 40, 50, 60, 70, 75, 80, 90, 100, 110, 120, 125, 150, 175, 200, 250, 300, 350, 400, 450, or 500 mg/L, or a yield in a range that is defined by any two of the preceding values. For example, in some embodiments, the expression yield of the antigen binding construct, minibody, or cys-diabody, is between about 1 and 500 mg/L, 1 and 350 mg/L, 1 and 200 mg/L, 1 and 100 mg/L, 1 and 50 mg/L, 1 and 25 mg/L, 5 and 500 mg/L, 5 and 350 mg/L, 5 and 200 mg/L, 5 and 100 mg/L, 5 and 50 mg/L, 5 and 25 mg/L, 25 and 500 mg/L, 25 and 350 mg/L, 25 and 20 mg/L, 25 and 100 mg/L, or 25 and 50 mg/L. In some embodiments, the expression yield of the antigen binding construct, minibody, and/or cys-diabody is determined with respect to the expression yield of the parental antigen binding construct, minibody, and/or cys-diabody. For example, in some embodiments, the expression yield of the antigen binding construct, minibody, or cys-diabody is about 1, 2, 3, 4, 5, 6, 7, 8, 9 or 10-fold higher than the expression yield of the parental antigen binding construct, minibody, and/or cys-diabody, or is increased by a range that is defined by any two of the preceding values. For example, in some embodiments, the expression yield of the antigen binding construct, minibody, and/or cys-diabody, is between 1 and 10-fold, 1 and 8-fold, 1 and 5-fold, 1 and 3-fold, 3 and 10-fold, 3 and 8-fold, 3 and 5-fold, 5 and 10-fold, or 5 and 8-fold higher than the expression yield of the parental antigen binding construct, minibody, and/or cys-diabody.

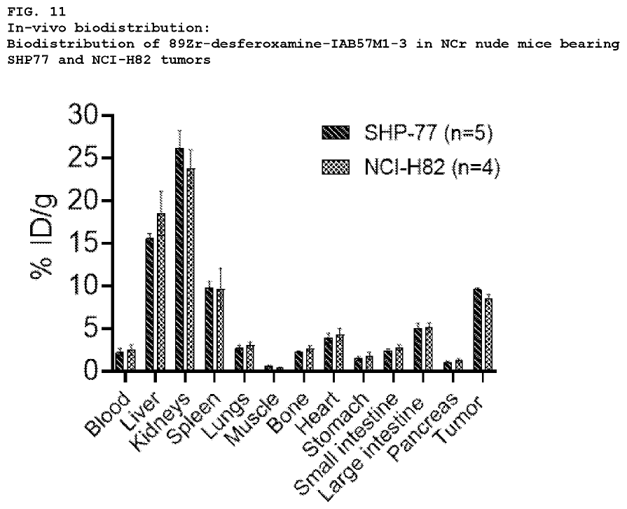

FIG. 11 is a bar graph showing some embodiments of the in-vivo biodistribution of an antigen binding minibody in tumor bearing mice models.

In some embodiments, the antigen binding construct, minibody, or cys-diabody, accumulates to a detectable level in a subject's blood, liver, kidney, spleen, lungs, muscle, bone, heart, stomach, small intestine, large intestine, pancreas, tumor, or any combination therein. In some embodiments, the antigen binding construct is biased for distribution to a subject's blood, liver, kidney, spleen, lungs, muscle, bone, heart, stomach, small intestine, large intestine, pancreas, tumor, or any combination therein.

In some embodiments, the antigen binding construct, minibody, or cys-diabody is detectable at, at about, up to, or up to about 0.1%, 0.5%, 1%, 2%, 3%, 4%, 5%, 6%, 7%, 8%, 9%, 10%, 15%, 20%, 25%, 30%, or 35% ID/g, or a percentage in a range that is defined by any two of the preceding values. For example, in some embodiments, the antigen binding construct, minibody, or cys-diabody, is detectable at between about 0.1% and 35%, 0.1% and 30%, 1% and 35%, 1% and 30%, 5% and 35%, or 5% and 30% ID/g.

In some embodiments, the antigen binding construct, minibody, and/or cys-diabody are distributed to the blood, liver, kidney, spleen, lungs, muscles, bones, heart, stomach, small intestine, large intestine, pancreas, tumor, or any combination therein. In some embodiments, the antigen binding construct, minibody, and/or cys-diabody are distributed to the blood, liver, kidney, spleen, lungs, muscles, bones, heart, stomach, small intestine, large intestine, pancreas, tumor, or any combination therein, at or at about 1%, 2%, 3%, 4%, 5%, 6%, 7%, 8%, 9%, 10%, 15%, 20%, 25%, 30%, 40%, 50%, 60%, 70%, 75%, 80%, 90%, 95%, or 100%, ID/g, or a percentage in a range that is defined by any two of the preceding values. For example, in some embodiments, the antigen binding construct, minibody, and/or cys-diabody are distributed to the blood, liver, kidney, spleen, lungs, muscles, bones, heart, stomach, small intestine, large intestine, pancreas, tumor, or any combination therein at an ID/g of between about 1% and 100%, 1% and 75%, 1% and 50%, 1% and 30%, 1% and 25%, 5% and 100%, 5% and 75%, 5% and 50%, 5% and 30%, 5% and 25%, 10% and 100%, 10% and 75%, 10% and 50%, 10% and 30%, or 10% and 25%.

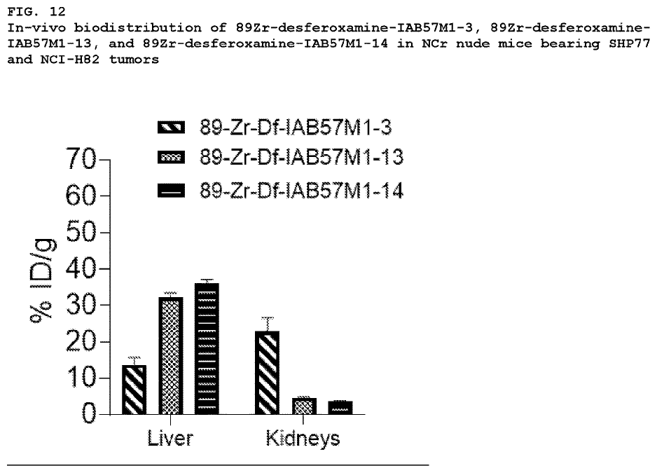

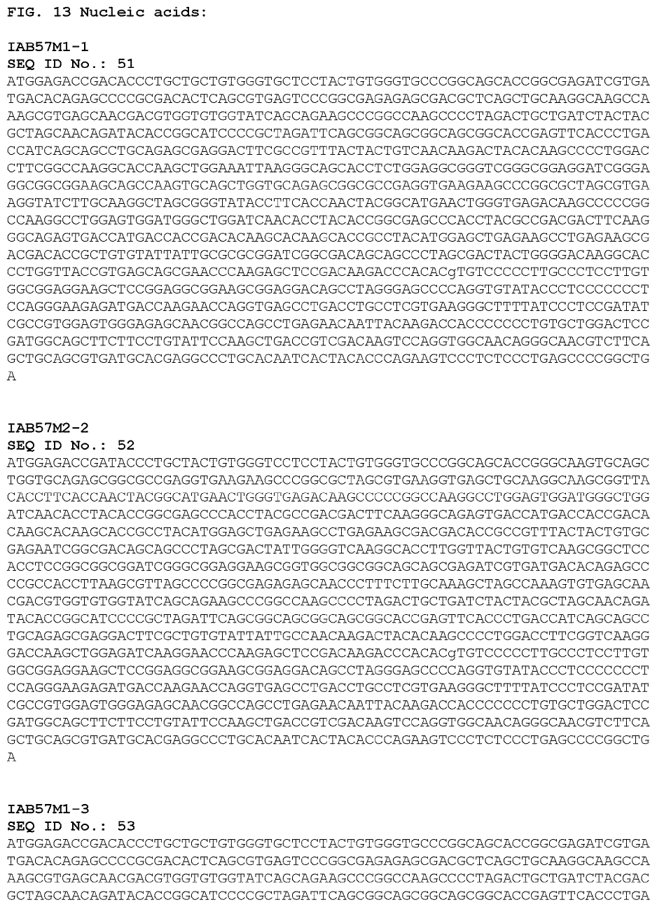

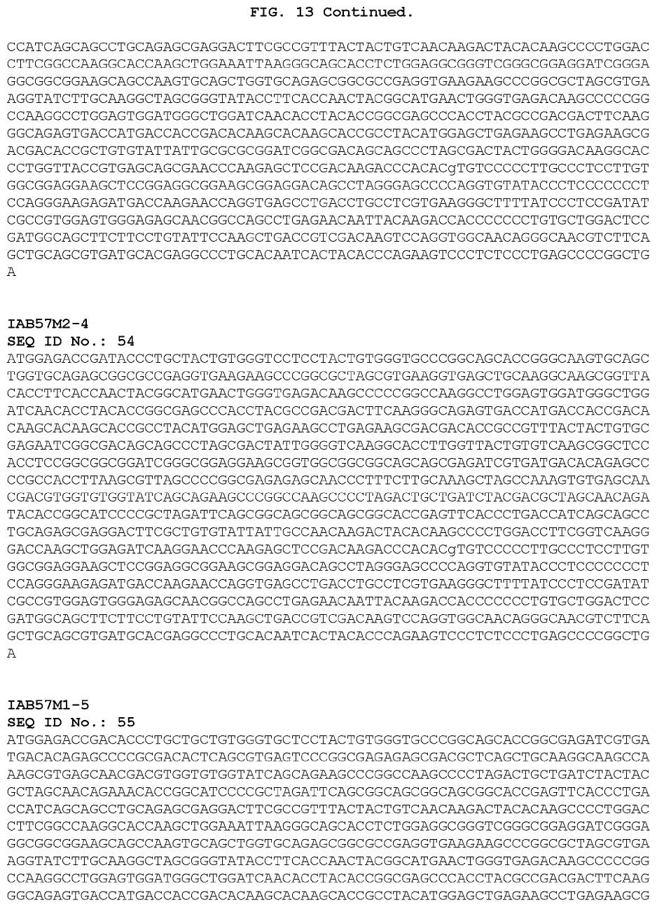

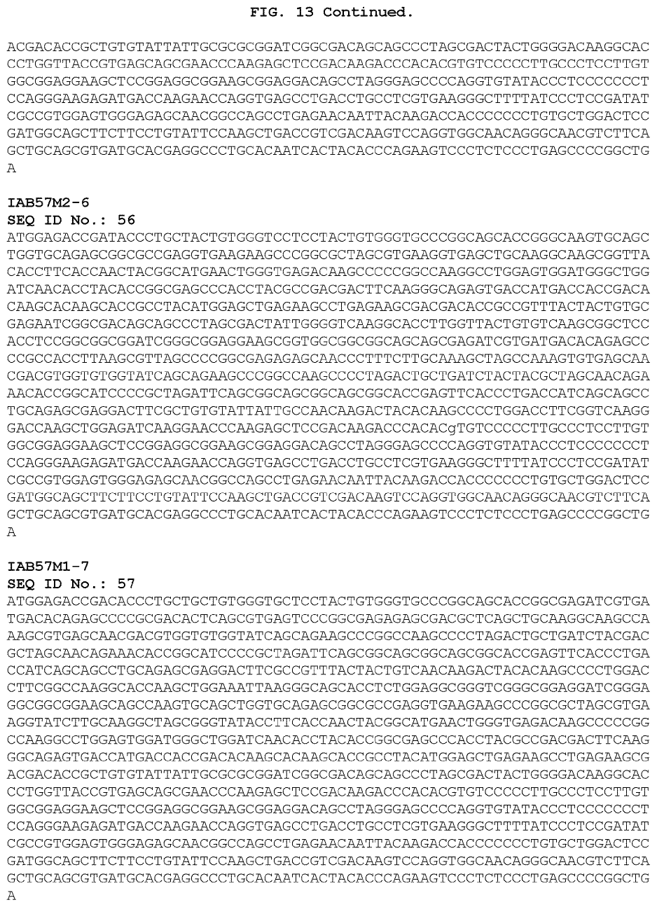

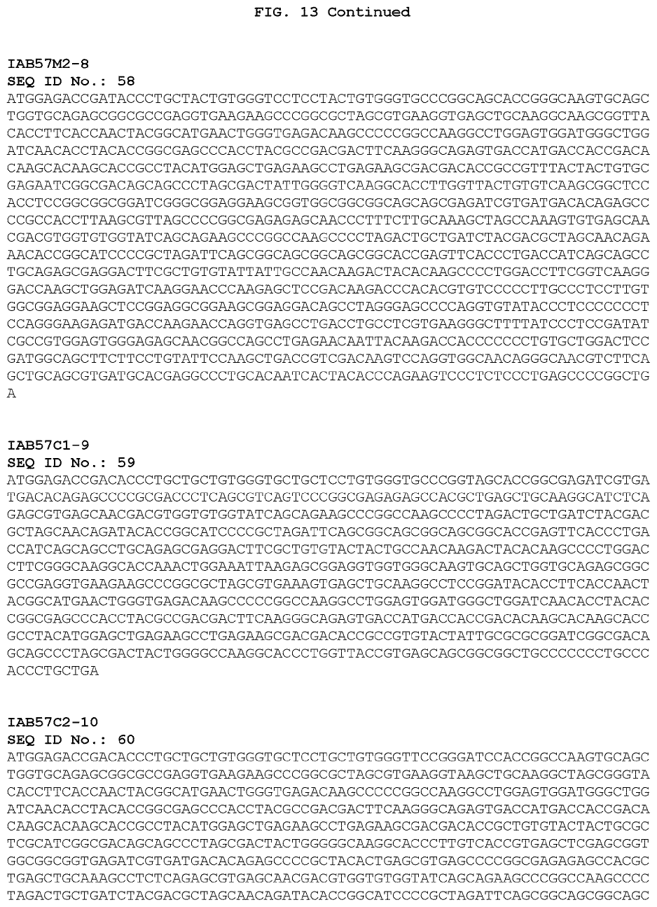

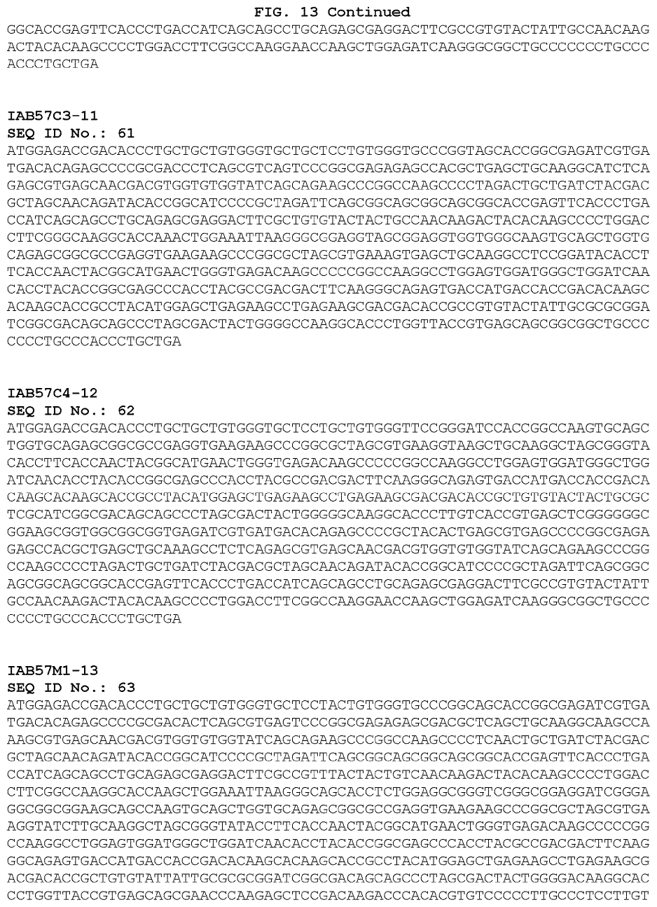

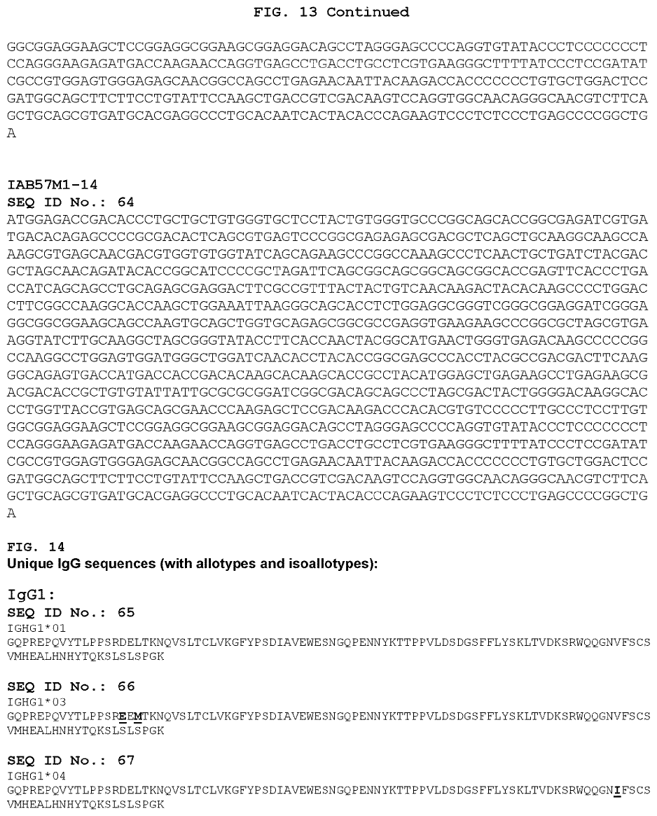

FIG. 12 is a bar graph showing some embodiments of the in-vivo biodistribution of antigen binding minibodies.

In some embodiments, the antigen binding construct, minibody, and/or cys-diabody are distributed to the blood, liver, kidney, spleen, lungs, muscles, bones, heart, stomach, small intestine, large intestine, pancreas, tumor, or any combination therein. In some embodiments, the antigen binding construct, minibody, and/or cys-diabody are distributed to the blood, liver, kidney, spleen, lungs, muscles, bones, heart, stomach, small intestine, large intestine, pancreas, tumor, or any combination therein, at or at about 1%, 2%, 3%, 4%, 5%, 6%, 7%, 8%, 9%, 10%, 15%, 20%, 25%, 30%, 40%, 50%, 60%, 70%, 75%, 80%, 90%, 95%, or 100%, ID/g, or a percentage in a range that is defined by any two of the preceding values. For example, in some embodiments, the antigen binding construct, minibody, and/or cys-diabody are distributed to the blood, liver, kidney, spleen, lungs, muscles, bones, heart, stomach, small intestine, large intestine, pancreas, tumor, or any combination therein at an ID/g of between about 1% and 100%, 1% and 75%, 1% and 50%, 1% and 30%, 1% and 25%, 5% and 100%, 5% and 75%, 5% and 50%, 5% and 30%, 5% and 25%, 10% and 100%, 10% and 75%, 10% and 50%, 10% and 30%, or 10% and 25%.

In some embodiments, the antigen binding construct, minibody, or cys-diabody, comprises a LFR2 that is SEQ ID NO: 35 or 37; and the antigen binding construct, minibody, or cys-diabody has improved biodistribution as compared to an antigen binding construct, minibody, or cys-diabody comprising an LFR2 that is SEQ ID NO: 33.

FIG. 9 is a graph showing some embodiments of equilibrium dissociation constant determinations of antigen binding minibodies.

In some embodiments, the antigen binding construct, minibody, or cys-diabody, has a KD (e.g., KD of binding to a DLL3 protein) of less than about 10⁻⁵ M, 10⁻⁶ M, 10⁻⁷ M, 10⁻¹ M, 10⁻⁹ M, 10⁻¹⁰ M, 10⁻¹¹ M, 10⁻¹² M, 10⁻¹ M, 10⁻¹⁴ M, or 10⁻¹⁵ M, or a KD in a range defined by any two of the preceding values. For example, in some embodiments, the antigen binding construct, minibody, or cys-diabody, has a KD (e.g., KD of binding to an antigen) of between about 10⁻⁵ M and 10⁻¹⁵ M, 10⁻⁵ M and 10⁻¹² M, 10⁻⁵ M and 10⁻¹¹ M, 10⁻⁷ M and 10⁻¹⁵ M, 10⁻⁷ M and 10⁻¹² M, 10⁻⁷ M and 10⁻¹⁰ M, 10⁻¹⁰ M and 10⁻¹⁵ M, or 10⁻¹⁰ M and 10⁻¹² M. In some embodiments, the KD is determined for an antibody dissolved in buffer. In some embodiments, the buffer is a phosphate buffer. If desired, the KD can be determined using any suitable option, e.g., biolayer interferometry.

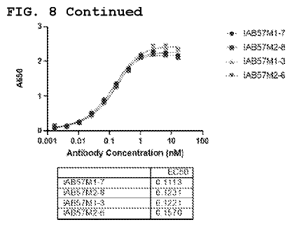

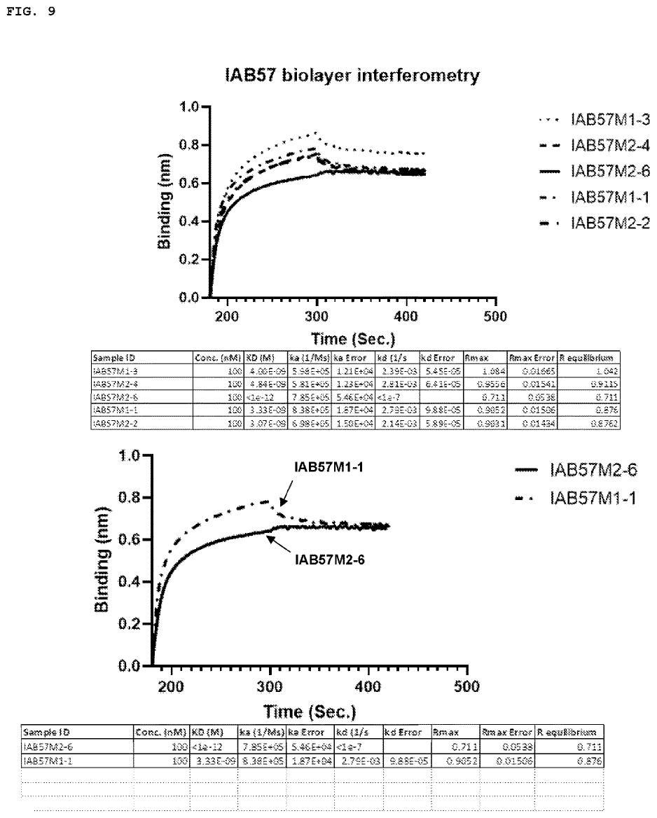

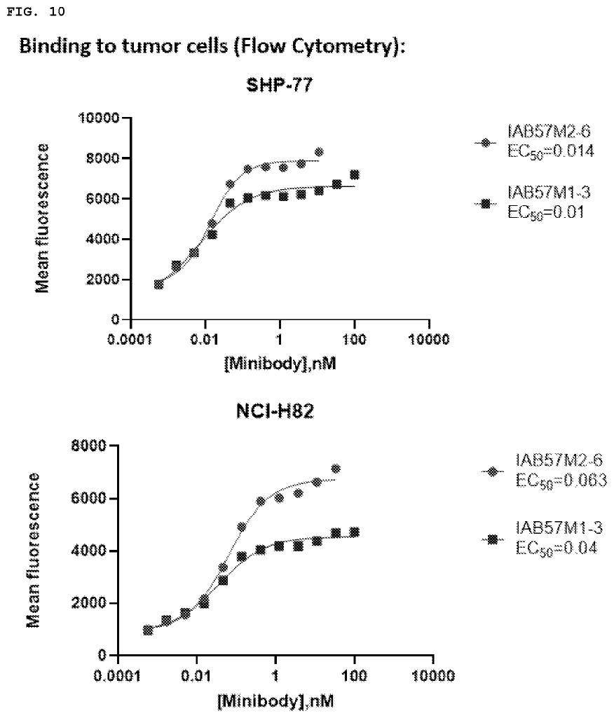

FIG. 8 is a graph showing some embodiments of EC₅₀ determinations of antigen binding minibodies by ELISA.

FIG. 10 is a graph showing some embodiments of EC₅₀ determinations of antigen binding minibodies by flow cytometry.

In some embodiments, the antigen binding construct, minibody, or cys-diabody, has an EC₅₀ (e.g., EC₅₀ of binding to a DLL3 protein, or a cell expressing a DLL3 protein) of, of about, of at most, or less than 0.001, 0.005, 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, or 0.20 nM, or an EC₅₀ in a range that is defined by any two of the preceding values. For example, in some embodiments, the antigen binding construct, minibody, or cys-diabody, has an EC₅₀ (e.g., EC₅₀ of binding to an antigen, or a cell expressing an antigen) of between about 0.001 and 0.20 nM, 0.001 and 0.17 nM, 0.001 and 0.12 nM, 0.01 and 0.20 nM, 0.01 and 0.17 nM, 0.01 and 0.12 nM, 0.05 and 0.20 nM, 0.05 and 0.17 nM. 0.05 and 0.15 nM, 0.05 and 0.12 nM, 0.08 and 0.20 nM, 0.08 and 0.17 nM, or 0.08 and 0.12 nM. In some embodiments, the antigen binding construct, minibody, or cys-diabody, has an EC₅₀ (e.g., EC₅₀ of binding to a DLL3 protein, or a cell expressing a DLL3 protein) of up to about 10 nM. If desired, the EC₅₀ can be determined using any suitable option, e.g., using ELISA or flow cytometry.

### Therapeutic Agents and Compositions

In some embodiments, the pharmaceutical composition can also include a pharmaceutically acceptable carrier. A pharmaceutically acceptable carrier can be a pharmaceutically acceptable material, composition, or vehicle that is involved in carrying or transporting a compound of interest from one tissue, organ, or portion of the body to another tissue, organ, or portion of the body. For example, the carrier can be a liquid or solid filler, diluent, excipient, solvent, or encapsulating material, or some combination thereof. Each component of the carrier is “pharmaceutically acceptable” in that it is compatible with the other ingredients of the formulation. It is also suitable for contact with any tissue, organ, or portion of the body that it can encounter, meaning that, ideally it will not carry a significant risk of toxicity, irritation, allergic response, immunogenicity, or any other complication that excessively outweighs its therapeutic benefits.

In some embodiments, the therapeutic composition comprises: an antigen binding construct that comprises: a variable light (V_{L}) domain comprising: a LCDR1 that is SEQ ID NO: 15; a LCDR2 that is any one of SEQ ID NO: 19, 21, or 23; a LCDR3 that is SEQ ID NO: 25; and a variable heavy (V_{H}) domain comprising: a HCDR1 that is SEQ ID NO: 27; a HCDR2 that is SEQ ID NO: 29; a HCDR3 that is SEQ ID NO: 31; and a therapeutic agent, toxic payload, and/or a detectable marker. In some embodiments, antigen binding construct is a minibody that further includes a hinge region (e.g., any one of the hinge regions described herein) and a IgG C_{H}3 sequence (e.g., any one of the C_{H}3 sequences described herein). In some embodiments, antigen binding construct is a cys-diabody that further includes an extension sequence (e.g., GGC or GGCPPCPPC (SEQ ID NO: 93)) at the C-terminus of a monomer.

In some embodiments, the therapeutic composition comprises: a minibody that binds to DLL3, the minibody comprising: a single-chain variable fragment (scFv) that binds to DLL3, the scFv comprising a variable light (V_{L}) domain linked to a variable heavy (V_{H}) domain, the V_{L} domain comprising: a LCDR1 that is SEQ ID NO: 15; a LCDR2 that is any one of SEQ ID NO: 19, 21, or 23; a LCDR3 that is SEQ ID NO: 25 and the V_{H} domain comprising: a HCDR1 that is SEQ ID NO: 27; a HCDR2 that is SEQ ID NO: 29; a HCDR3 that is SEQ ID NO: 31; a hinge-extension domain comprising a IgG1 hinge region; a IgG C_{H}3 sequence; and a therapeutic agent, toxic payload, and/or a detectable marker. In some embodiments, the therapeutic composition comprises: a minibody that binds to DLL3, the minibody comprising: a single-chain variable fragment (scFv) that binds to DLL3, the scFv comprising a variable light (V_{L}) domain linked to a variable heavy (V_{H}) domain, the V_{L} domain comprising: a LCDR1 that is SEQ ID NO: 15; a LCDR2 that is any one of SEQ ID NO: 19, 21, or 23; a LCDR3 that is SEQ ID NO: 25 and the V_{H} domain comprising: a HCDR1 that is SEQ ID NO: 27; a HCDR2 that is SEQ ID NO: 29; a HCDR3 that is SEQ ID NO: 31; a hinge region; a IgG C_{H}3 sequence; and a therapeutic agent, toxic payload, and/or a detectable marker.

In some embodiments, the therapeutic composition comprises: a cys-diabody that binds to DLL3, the cys-diabody comprising a polypeptide that comprises: a single-chain variable fragment (scFv) comprising a variable light (V_{L}) domain linked to a variable heavy (V_{H}) domain; the V_{L} domain comprising: a LCDR1 of LCDR1 of SEQ ID NO: 15; a LCDR2 of a LCDR2 of SEQ ID NO: 19, 21, or 23; a LCDR3 of a LCDR3 of SEQ ID NO: 25; the V_{H} domain comprising: a HCDR1 of a HCDR1 of SEQ ID NO: 27; a HCDR2 of a HCDR2 of SEQ ID NO: 29; a HCDR3 of a HCDR3 of SEQ ID NO: 31; and a therapeutic agent, toxic payload, and/or a detectable marker. In some embodiments, the therapeutic composition comprises: a cys-diabody that binds to DLL3, the cys-diabody comprising a polypeptide that comprises: a single-chain variable fragment (scFv) comprising a variable light (V_{L}) domain linked to a variable heavy (V_{H}) domain; the V_{L} domain comprising: a LCDR1 of LCDR1 of SEQ ID NO: 15; a LCDR2 of a LCDR2 of SEQ ID NO: 19, 21, or 23; a LCDR3 of a LCDR3 of SEQ ID NO: 25; a V_{H} domain comprising: a HCDR1 of a HCDR1 of SEQ ID NO: 27; a HCDR2 of a HCDR2 of SEQ ID NO: 29; a HCDR3 of a HCDR3 of SEQ ID NO: 31; an extension sequence; and a therapeutic agent, toxic payload, and/or a detectable marker.

In some embodiments, the therapeutic composition comprises one or more of the minibody arrangements provided in any one of the figures and/or in the Tables, such as Table 5.

In some embodiments, the therapeutic composition comprises one or more of the cys-diabodies arrangements provided in any one of the figures and/or in the Tables, such as Table 6.

In some embodiments, the therapeutic composition comprises an antigen binding constructs, minibodies, cys-diabodies, V_{L}, V_{H}, CDR, FR, linker, signal peptide, or any combination therein, having at least about 70%, 75%, 80%, 85%, 90%, 95%, 96%, 97%, 98% or 99% sequence identity to an antigen binding construct, minibody, cys-diabody, V_{L}, V_{H}, CDR, FR, linker, signal peptide, of any of one or more of SEQ ID NOs: 1-48. In some embodiments, the therapeutic composition comprises a minibody that is any of SEQ ID NOs: 1-10. In some embodiments, the therapeutic composition comprises a minibody having at least about 70%, 75%, 80%, 85%, 90%, 95%, 96%, 97%, 98% or 99% sequence identity to a minibody that is any of SEQ ID NOs: 1-10. In some embodiments, the therapeutic composition comprises a cys-diabody that is any of SEQ ID NOs: 11-14. In some embodiments, the therapeutic composition comprises a cys-diabody having at least about 70%, 75%, 80%, 85%, 90%, 95%, 96%, 97%, 98% or 99% sequence identity to a cys-diabody that is any of SEQ ID NOs: 11-14.

In some embodiments, the therapeutic composition comprises a LCDR that is any of SEQ ID NOs: 15-26. In some embodiments, the therapeutic composition comprises a LCDR having at least about 70%, 75%, 80%, 85%, 90%, 95%, 96%, 97%, 98% or 99% sequence identity to a LCDR that is any of SEQ ID NO: 15-26. In some embodiments, the therapeutic composition comprises a HCDR that is any of SEQ ID NOs: 27-32. In some embodiments, the therapeutic composition comprises a HCDR having at least about 70%, 75%, 80%, 85%, 90%, 95%, 96%, 97%, 98% or 99% sequence identity to a HCDR that is any of SEQ ID NOs: 27-32. In some embodiments, the therapeutic composition comprises a FR that is any of SEQ ID NOs: 33-38. In some embodiments, the therapeutic composition comprises a FR having at least about 70%, 75%, 80%, 85%, 90%, 95%, 96%, 97%, 98% or 99% sequence identity to a FR that is any of SEQ ID NOs: 33-38. In some embodiments, the therapeutic composition comprises a linker that is any of SEQ ID NO: 39-44. In some embodiments, the therapeutic composition comprises a linker having at least about 70%, 75%, 80%, 85%, 90%, 95%, 96%, 97%, 98% or 99% sequence identity to a linker that is any of SEQ ID NO: 39-44. In some embodiments, the therapeutic composition comprises a signal peptide that is any of SEQ ID NOs: 45-46. In some embodiments, the therapeutic composition comprises a signal peptide having at least about 70%, 75%, 80%, 85%, 90%, 95%, 96%, 97%, 98% or 99% sequence identity to a signal peptide that is any of SEQ ID NOs: 45-46. In some embodiments, the therapeutic composition comprises a V_{H} that is SEQ ID NO: 47. In some embodiments, the therapeutic composition comprises a minibody having at least about 70%, 75%, 80%, 85%, 90%, 95%, 96%, 97%, 98% or 99% sequence identity to a V_{H} that is SEQ ID NO: 47. In some embodiments, the therapeutic composition comprises a V_{L} that is SEQ ID NO: 48. In some embodiments, the therapeutic composition comprises a V_{L} having at least about 70%, 75%, 80%, 85%, 90%, 95%, 96%, 97%, 98% or 99% sequence identity to a V_{L} that is any of SEQ ID NO: 48.

The pharmaceutical, or therapeutic, compositions described herein can be administered by any suitable route of administration. A route of administration can refer to any administration pathway known in the art, including but not limited to aerosol, enteral, nasal, ophthalmic, oral, parenteral, rectal, transdermal (e.g., topical cream or ointment, patch), or vaginal. “Transdermal” administration can be accomplished using a topical cream or ointment or by means of a transdermal patch. “Parenteral” refers to a route of administration that is generally associated with injection, including infraorbital, infusion, intraarterial, intracapsular, intracardiac, intradermal, intramuscular, intraperitoneal, intrapulmonary, intraspinal, intrasternal, intrathecal, intracranial, intraventricular, intrauterine, intravenous, subarachnoid, subcapsular, subcutaneous, sublingual, transmucosal, or transtracheal. In some embodiments, the antigen binding construct can be delivered intraoperatively as a local administration during an intervention or resection.

In some embodiments, an antigen binding construct, minibody, or cys-diabody, is conjugated to a therapeutic agent. A “therapeutic agent” as used herein is an atom, molecule, or compound that is useful in the treatment of a disorder related to a target molecule. Examples of therapeutic agents include, but are not limited to, drugs, chemotherapeutic agents, therapeutic antibodies and antibody fragments, toxins, radioisotopes, enzymes (for example, enzymes to cleave prodrugs to a cytotoxic agent at the site of the antigen binding construct binding), nucleases, hormones, immunomodulators, antisense oligonucleotides, chelators, boron compounds, photoactive agents and dyes, elastin-like polypeptides such as PLGA, and nanoparticles. Examples of disorders include those related to one or more target molecules.

Chemotherapeutic agents are often cytotoxic or cytostatic in nature and may include alkylating agents, antimetabolites, anti-tumor antibiotics, topoisomerase inhibitors, mitotic inhibitors hormone therapy, targeted therapeutics and immunotherapeutics. In some embodiments the chemotherapeutic agents that may be used as detectable markers in accordance with the embodiments of the disclosure include, but are not limited to, 13-cis-Retinoic Acid, 2-Chlorodeoxyadenosine, 5-Azacitidine, 5-Fluorouracil, 6-Mercaptopurine, 6-Thioguanine, actinomycin-D, adriamycin, aldesleukin, alemtuzumab, alitretinoin, all-transretinoic acid, alpha interferon, altretamine, amethopterin, amifostine, anagrelide, anastrozole, arabinosylcytosine, arsenic trioxide, amsacrine, aminocamptothecin, aminoglutethimide, asparaginase, azacytidine, bacillus calmette-guerin (BCG), bendamustine, bevacizumab, bexarotene, bicalutamide, bortezomib, bleomycin, busulfan, calcium leucovorin, citrovorum factor, capecitabine, canertinib, carboplatin, carmustine, cetuximab, chlorambucil, cisplatin, cladribine, cortisone, cyclophosphamide, cytarabine, darbepoetin alfa, dasatinib, daunomycin, decitabine, denileukin diftitox, dexamethasone, dexasone, dexrazoxane, dactinomycin, daunorubicin, decarbazine, docetaxel, doxorubicin, doxifluridine, eniluracil, epirubicin, epoetin alfa, erlotinib, everolimus, exemestane, estramustine, etoposide, filgrastim, fluoxymesterone, fulvestrant, flavopiridol, floxuridine, fludarabine, fluorouracil, flutamide, gefitinib, gemcitabine, gemtuzumab ozogamicin, goserelin, granulocyte-colony stimulating factor, granulocyte macrophage-colony stimulating factor, hexamethylmelamine, hydrocortisone hydroxyurea, ibritumomab, interferon alpha, interleukin-2, interleukin-11, isotretinoin, ixabepilone, idarubicin, imatinib mesylate, ifosfamide, irinotecan, lapatinib, lenalidomide, letrozole, leucovorin, leuprolide, liposomal Ara-C, lomustine, mechlorethamine, megestrol, melphalan, mercaptopurine, mesna, methotrexate, methylprednisolone, mitomycin C, mitotane, mitoxantrone, nelarabine, nilutamide, octreotide, oprelvekin, oxaliplatin, paclitaxel, pamidronate, pemetrexed, panitumumab, PEG Interferon, pegaspargase, pegfilgrastim, PEG-L-asparaginase, pentostatin, plicamycin, prednisolone, prednisone, procarbazine, raloxifene, rituximab, romiplostim, ralitrexed, sapacitabine, sargramostim, satraplatin, sorafenib, sunitinib, semustine, streptozocin, tamoxifen, tegafur, tegafur-uracil, temsirolimus, temozolamide, teniposide, thalidomide, thioguanine, thiotepa, topotecan, toremifene, tositumomab, trastuzumab, tretinoin, trimitrexate, alrubicin, vincristine, vinblastine, vindestine, vinorelbine, vorinostat, or zoledronic acid.

Toxins that may be used in accordance with the embodiments of the disclosure include, but are not limited to, Auristatin E, Auristatin F, Dolastatin 10, Dolastatin 15, combretastatin and their analogs, maytansinoid, calicheamicin, alpha-amanitin, pyrrolobenzodiazepine dimers, epothilones, duocarmycin and their analogs, tubulysin D, basillistatins, ricin, abrin, ribonuclease (RNase), DNase I, Staphylococcal enterotoxin-A, pokeweed antiviral protein, gelonin, diphtheria toxin, Pseudomonas exotoxin, and Pseudomonas endotoxin.

In some embodiments nanoparticles are used in therapeutic applications as drug carriers that, when conjugated to an antigen binding construct, minibody, or cys-diabody, deliver chemotherapeutic agents, hormonal therapeutic agents, radiotherapeutic agents, toxins, or any other cytotoxic or anti-cancer agents known in the art to cancerous cells that overexpress the target on the cell surface.

In some embodiments, the antigen binding constructs, minibodies, or cys-diabodies described herein may be further conjugated with one or more immunotherapeutic agents. In some embodiments, the antigen binding constructs, minibodies, or cys-diabodies described herein may be co-administered with one or more immunotherapeutic agents. For example, in some embodiments, the antigen binding constructs, minibodies, or cys-diabodies described herein may be further conjugated, or co-administered, with anti-PD1 and anti-PD-Li binding agents, anti-CTLA4 agents, and multi-specific agents including, but not limited to, anti-CTLA-4/B7-1/B7-2. Additional immunotherapies include checkpoint inhibitors such as ipilimumab (Yervoy), pembrolizumab (Keytruda), nivolumab (Opdivo), atezolizumab (Tecentriq), avelumab (Bavencio), and durvalumab (Imfinzi). Immunotherapies also include tremelimumab and pidilizumab. Small molecule immunotherapies are also in development including BMS-1001, BMS-1116, CA-170, CA-327, Imiquimod, Resiquimod, 852A, VTX-2337, ADU-S100, MK-1454, Ibrutinib, 3AC, Idelalisib, IPI-549, Epacadostat, AT-38, CPI-444, Vipadenant, Preladenant, PBF, AZD4635, Galuniseritib, OTX015/MK-8628, CPI-0610 (c.f. Kerr and Chisolm (2019) The Journal of Immunology, 2019, 202: 11-19.)

In some embodiments, the antigen binding constructs, minibodies, or cys-diabodies described herein may be further conjugated with one or more kidney- and/or radioprotecting agents. In some embodiments, the antigen binding constructs, minibodies, or cys-diabodies described herein may be co-administered with one or more kidney- and/or radioprotecting agents. In some embodiments, the radioprotecting agent comprises free lysine, free arginine, probenecid, gelofusin, 2,4-dinitrophenol, caffeine, Ibuprofen, ascorbic acid, caffeic acid, aspirin, carnosine, minocycline, catechin, 4′-O-methylcatechin, 4-phenylbutyric acid, 14937-32-7, lithium chloride, cyclosporine, epoprostenol, fullerene, fumarate, gallic acid, metformin, indirubin, kaempferol, sodium butyrate, spermidine, cholecalciferol, avobenzone, octinoxate, titanium dioxide, tempol, gusperimus, irsogladine, tetrachlorodecaoxide, calcium alginate, gusperimus hydrochloride, D-galacturonic acid sodium salt, cystamine hydrobromide, cystaphos, N-Acetyl-Ser-Asp-Lys-Pro, D08Eai, prolycopene, velaresol, U-74389G maleate, trans-2-hexenyl salicylate, 3-benzylidene camphor, irsogladine maleate, sodium alginate, Gsh-Mee, triethylenediamine diacetate, cystamine hydrochloride, amifostine, glutathione reduced ethyl ester, homocysteine thiolactone, 132316-35-9, hydroquinone, 13258-59-8, adeturon, unii-S8Kc806H2T, nitrogen oxygen, 2-ethyl-3-hydroxy-6-methylpyridine, S-ethylglutathione, (8S,10S,13S,14S,17S)-17-[2-[4-(2,6-dipyrrolidin-1-ylpyrimidin-4-Yl)piperazin-1-Yl]acetyl]-10,13-dimethyl-6,7,8,12,14,15,16,17-actahydrocyclopenta[A]phenanthren-3-One, trilazad mesylate, Wr 1065, posphonol, amifostine, 1,4-diazabicyclo[2.2.2]octane, gusperimus trihydrochloride, tributyl phosphate, sulfolane, deuterium oxide, benzobarbital, 6-methyluracil, homosalate, cystamine dihydrochloride, 2-(2-aminoethyl)isothiourea dihydrobromide, tempol-H, fenofibrate, cystamine, beta-aminoethyl isothiourea, zingerone, aeol 10150, artemisinin, 16,16-dimethylprostaglandin E2, theaflavin, succinic acid, andrographis, balsalazide, N-adenylyl-L-phenylalanine, chlorophyllin, nacu, dieckol, eckol, diltiazem, edaravone, rhepo, ferulic acid, glutathione, glycyrrhizic acid, hesperidin, indomethacin, misoprostol, morin, myricetin, tiopronin, naringin, recilisib sodium, probucol, hydroxyethylrutoside, cystaphos, sulfasalazine, sucralfate, chugai, silymarin, floxacillin, cyproterone, cysteamine, thymol, mercaptoethanol, desogestrel, cellobiose, kanamycin, sodium alginate, acetylcysteine, zinc oxide, cystaphos, 10-hydroxy-2-decenoic acid, teduglutide, curcuma, troxerutin, trichostatin A, selegiline, lycopene, nitrendipine, glycerol, 21245-02-3, rosmarinic acid, simvastatin, fisetin, sirolimus, oxybenzone, ascophyllum, ecamsule, Fsfjezphmoqyqb-Oasotcbpsa-N, Sczygarxjgtndn-Ikfjuqjosa-N, Snzlftjkilzfkf-Uhfffaoysa-M, alginate, alginic acid, potassium salt, potassium alginate, U-74389G, calcium alginate, Hoechst 33342 (Trihydrochloride), polydatin, thioctic acid, enalapril, amifostine, ursolic acid, Unii-Sm5Yj88Ltu, carbonyl cyanide m-chlorophenyl hydrazone, genistein, resveratrol, (−)-epigallocatechin gallate, ellagic acid, baicalein, valproic acid, pentoxifylline, acacia, melatonin, trehalose, acteoside, kukoamine, atorvastatin, carvacrol, isofraxidin, palifermin, cysteine, vitamin E, heparin, chondroethin sulfate, AET, M40403, EUK-189, EUK-207, vitamin C, vitamin A, lipoic acid, β-carotene, L-selenomethionine, and/or Q10.

Any of the antigen binding constructs, minibodies, or cys-diabodies described herein may be further conjugated with one or more additional therapeutic agents, detectable markers, nanoparticles, carriers or a combination thereof. For example, an antigen binding construct may be radiolabeled with Iodine-131 and conjugated to a lipid carrier, such that the anti-target molecule-lipid conjugate forms a micelle. The micelle can incorporate one or more therapeutic agents, isotopes, ions, and/or detectable markers.

In some embodiments, antigen binding constructs, minibodies, or cys-diabodies are conjugated to a therapeutic agent. While these antigen binding constructs can have a shorter circulation half-life compared to a full-length antibody, in some embodiments, these formats can exhibit improved tumor penetration based on their smaller size and be therapeutically effective when appropriately armed with a cytotoxic drug or radioisotope. In some embodiments, an antigen binding construct, minibody, or cys-diabody, drug-conjugate approach can be employed. In some embodiments, a therapeutic approach includes radioimmunotherapy by attaching an appropriate radiolabel such as, Iodine-131, a beta-emitter or alpha-emitter, such as, Yttrium-90, Lutetium-177, Copper-67, Terbium-149, Terbium-161, Astatine-211, Lead-212, Bismuth-212, Actinium-225, Bismuth-213, and 227-Thorium, which can deliver cell damage and death to a target tissue. In some embodiments, treatment with these fragments armed with a cytotoxic drug or radionuclide result in less nonspecific toxicity as they will be cleared from the body more rapidly. In some embodiments, the radiolabel includes Terbium-149, Terbium-161, or Lead-212.

In some embodiments, the label and/or therapeutic agent comprises ¹⁸F, ¹⁸F-FAC, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁵Sc, ⁷⁷As, ⁸⁶Y, ⁹⁰Y, ⁸⁹Sr, ⁸⁹Zr, ⁹⁴Tc, ⁹⁴Tc, ⁹⁹mTc, ⁹⁹Mo, ¹⁰⁵Pd, ¹⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁴⁹Tb, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁷Th and ²²⁵Ac, or any combination thereof.

In some embodiments, the antigen binding construct, minibody, or cys-diabody, can be connected to a therapeutic agent to a disorder associated with the expression of the target molecule.

In some embodiments, target molecule antigen binding constructs are used as a stand-alone medicament (e.g. antigen binding construct in the absence of therapeutic agent) in the treatment of a disorder associated with the expression of the target molecule.

### Methods of Detecting the Presence or Absence of the Target Molecule

Antigen binding constructs, minibodies, or cys-diabodies can be used to detect the presence or absence of the target molecule in vivo and/or in vitro. Accordingly, some embodiments include methods of detecting the presence or absence of the target.

In some embodiments, the antigen binding construct, minibody, or cys-diabody, is labeled with a detectable marker. The marker can be for instance, a radioisotope, a fluorescent compound, a bioluminescent compound, chemiluminescent compound, a metal chelator or an enzyme. In some embodiments, the detectable marker comprises a phototherapy dye. In some embodiments, the detectable marker comprises boron. In some embodiments, the detectable marker comprises a marker that is compatible with Boron Neutron Capture Therapy (BNCT). In some embodiments, the detectable marker is suitable for use with Auger electron spectroscopy. In some embodiments, the detectable marker comprises a payload. In some embodiments, the at least one payload is selected from a group consisting of ¹⁸F, ¹⁸F-FAC, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁵Sc, ⁷⁷As, ⁸⁶Y, ⁹⁰Y, ⁸⁹Sr, ⁸⁹Zr, ⁹⁴Tc, ⁹⁴Tc, ⁹⁹mTc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm ¹⁴⁹Tb, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴I, ¹⁹⁸Au, ¹⁹⁹Au, ²¹¹At, ²¹¹Pb, ²¹²Bi, ²¹²Pb, ²¹³Bi, ²²³Ra, ²²⁷Th and ²²⁵Ac, or any combination thereof. In some embodiments, the marker is a radiolabel. Many radionucleotides may be used as imaging labels, including without limitation, ²¹¹At, ¹³¹I, ¹²⁵I, ⁹⁰Y, ¹⁸⁶Re, ¹⁸⁸Re, ¹⁵³Sm, ²¹²Bi, ³²P, ²¹²Pb, ⁸⁹Zr, ⁶⁴Cu, ¹⁸F, ⁶⁸G, ¹²⁴I, and radioactive isotopes of Lu and the like. One of skill in the art will know of other radionuclides particularly well suited for use in the present disclosure.

The method can kill the cancer cell. In some embodiments, the construct recognizes and binds the DLL3 protein. In additional embodiments, the method further comprises administering to a chemotherapeutic drug, radiation therapy. In some embodiments, the subject is also treated with hormone ablation therapy or hormone antagonist therapy.

The method can include applying an antigen binding construct, minibody, or cys-diabody, to a subject. In some embodiments, the antigen binding construct, minibody, or cys-diabody, is applied to a sample. The method can include detecting a binding or an absence of binding of the antigen binding construct, minibody, or cys-diabody, to the target molecule. In some embodiments, any target molecule could be detected through the options provided herein. In some embodiments, the target molecule to be detected is DLL3. In some embodiments, the target molecule is detected using one or more of the minibody arrangements provided in the figures and/or in the Tables, such as Table 5.

Table 5 provides embodiments which are useful as compositions and in the methods described herein:

TABLE 5

| Name | Sequence |
|---|---|
| IAB57M1-1 | METDTLLLWVLLLWVPGSTGEIVMTQSPATLSVSPGERATLSCKASQSV |
| | SNDVVWYQQKPGQAPRLLIYYASNRYTGIPARFSGSGSGTEFTLTISSL |
| | QSEDFAVYYCQQDYTSPWTFGQGTKLEIKGSTSGGGSGGGSGGGGSSQV |
| | QLVQSGAEVKKPGASVKVSCKASGYTFTNYGMNWVRQAPGQGLEWMGWI |
| | NTYTGEPTYADDFKGRVTMTTDISTSTAYMELRSLRSDDTAVYYCARIG |
| | DSSPSDYWGQGTLVTVSSEPKSSDKTHTCPPCPPCGGGSSGGGSGGQPR |
| | EPQVYTLPPSREEMTKNQVSLTCLVKGFYPSDIAVEWESNGQPENNYKT |
| | TPPVLDSDGSFFLYSKLTVDKSRWQQGNVFSCSVMHEALHNHYTQKSLS |
| | LSPG |
| | (SEQ ID NO: 1) |
| IAB57M2-2 | METDTLLLWVLLLWVPGSTG |
| | QVQLVQSGAEVKKPGASVKVSCKASGYTFTNYGMNWVRQAPGQGLEWMG |
| | WINTYTGEPTYADDFKGRVTMITDTSTSTAYMELRSLRSDDTAVYYCAR |
| | IGDSSPSDYWGQGILVTVSSGSTSGGGSGGGSGGGGSSEIVMTQSPATL |
| | SVSPGERATLSCKASQSVSNDVVWYQQKPGQAPRLLIYYASNRYTGIPA |
| | RFSGSGSGTEFTLTISSLQSEDFAVYYCQQDYTSPWTFGQGTKLEIKEP |
| | KSSDKTHTCPPCPPCGGGSSGGGSGGQPREPQVYTLPPSREEMTKNQVS |
| | LTCLVKGFYPSDIAVEWESNGQPENNYKTTPPVLDSDGSFFLYSKLTVD |
| | KSRWQQGNVFSCSVMHEALHNHYTQKSLSLSPG |
| | (SEQ ID NO: 2) |
| IAB57M1-3 | METDTLLLWVLLLWVPGSTG |
| | EIVMTQSPATLSVSPGERATLSCKASQSVSNDVVWYQQKPGQAPRLLIY |
| | DASNRYTGIPARFSGSGSGTEFTLTISSLQSEDFAVYYCQQDYTSPWTF |
| | GQGTKLEIKGSTSGGGSGGGSGGGGSSQVQLVQSGAEVKKPGASVKVSC |
| | KASGYTFTNYGMNWVRQAPGQGLEWMGWINTYTGEPTYADDFKGRVTMT |
| | TDTSTSTAYMELRSLRSDDTAVYYCARIGDSSPSDYWGQGTLVTVSSEP |
| | KSSDKTHTCPPCPPCGGGSSGGGSGGQPREPQVYTLPPSREEMTKNQVS |
| | LTCLVKGFYPSDIAVEWESNGQPENNYKTTPPVLDSDGSFFLYSKLTVD |
| | KSRWQQGNVFSCSVMHEALHNHYTQKSLSLSPG |
| | (SEQ ID NO: 3) |
| IAB57M2-4 | METDTLLLWVLLLWVPGSTG |
| | QVQLVQSGAEVKKPGASVKVSCKASGYTFTNYGMNWVRQAPGQGLEWMG |
| | WINTYTGEPTYADDFKGRVTMTTDTSTSTAYMELRSLRSDDTAVYYCAR |
| | IGDSSPSDYWGQGTLVTVSSGSTSGGGSGGGSGGGGSSEIVMTQSPATL |
| | SVSPGERATLSCKASQSVSNDVVWYQQKPGQAPRLLIYDASNRYTGIPA |
| | RFSGSGSGTEFTLTISSLQSEDFAVYYCQQDYTSPWTFGQGTKLEIKEP |
| | KSSDKTHTCPPCPPCGGGSSGGGSGGQPREPQVYTLPPSREEMTKNQVS |
| | LTCLVKGFYPSDIAVEWESNGQPENNYKTTPPVLDSDGSFFLYSKLTVD |
| | KSRWQQGNVF SCSVMHEALHNHYTQKSLSLSPG |
| | (SEQ ID NO: 4) |
| IAB57M1-5 | METDTLLLWVLLLWVPGSTG |
| | EIVMTQSPATLSVSPGERATLSCKASQSVSNDVVWYQQKPGQAPRLLIY |
| | YASNRNTGIPARFSGSGSGTEFTLTISSLQSEDFAVYYCQQDYTSPWTF |
| | GQGTKLEIKGSTSGGGSGGGSGGGGSSQVQLVQSGAEVKKPGASVKVSC |
| | KASGYTFTNYGMNWVRQAPGQGLEWMGWINTYTGEPTYADDFKGRVTMT |
| | TDTSTSTAYMELRSLRSDDTAVYYCARIGDSSPSDYWGQGTLVTVSSEP |
| | KSSDKTHTCPPCPPCGGGSSGGGSGGQPREPQVYTLPPSREEMTKNQVS |
| | LTCLVKGFYPSDIAVEWESNGQPENNYKTTPPVLDSDGSFFLYSKLTVD |
| | KSRWQQGNVFSCSVMHEALHNHYTQKSLSLSPG |
| | (SEQ ID NO: 5) |
| IAB57M2-6 | METDILLLWVLLLWVPGSTG |
| | QVQLVQSGAEVKKPGASVKVSCKASGYTFTNYGMNWVRQAPGQGLEWMG |
| | WINTYTGEPTYADDFKGRVTMTTDTSTSTAYMELRSLRSDDTAVYYCAR |
| | IGDSSPSDYWGQGILVTVSSGSTSGGGSGGGSGGGGSSEIVMTQSPATL |
| | SVSPGERATLSCKASQSVSNDVVWYQQKPGQAPRLLIYYASNRNIGIPA |
| | RFSGSGSGTEFTLTISSLQSEDFAVYYCQQDYTSPWTFGQGTKLEIKEP |
| | KSSDKTHTCPPCPPCGGGSSGGGSGGQPREPQVYTLPPSREEMTKNQVS |
| | LTCLVKGFYPSDIAVEWESNGQPENNYKTTPPVLDSDGSFFLYSKLTVD |
| | KSRWQQGNVFSCSVMHEALHNHYTQKSLSLSPG |
| | (SEQ ID NO: 6) |
| IAB57M1-7 | METDTLLLWVLLLWVPGSTG |
| | EIVMTQSPATLSVSPGERATLSCKASQSVSNDVVWYQQKPGQAPRLLIY |
| | DASNRNTGIPARFSGSGSGTEFTLTISSLQSEDFAVYYCQQDYTSPWTF |
| | GQGTKLEIKGSTSGGGSGGGSGGGGSSQVQLVQSGAEVKKPGASVKVSC |
| | KASGYTFTNYGMNWVRQAPGQGLEWMGWINTYTGEPTYADDFKGRVTMT |
| | TDISTSTAYMELRSLRSDDTAVYYCARIGDSSPSDYWGQGTLVTVSSEP |
| | KSSDKTHTCPPCPPCGGGSSGGGSGGQPREPQVYTLPPSREEMTKNQVS |
| | LTCLVKGFYPSDIAVEWESNGQPENNYKTTPPVLDSDGSFFLYSKLTVD |
| | KSRWQQGNVFSCSVMHEALHNHYTQKSLSLSPG |
| | (SEQ ID NO: 7) |
| IAB57M2-8 | METDTLLLWVLLLWVPGSTG |
| | QVQLVQSGAEVKKPGASVKVSCKASGYTFTNYGMNWVRQAPGQGLEWMG |
| | WINTYTGEPTYADDFKGRVTMTTDTSTSTAYMELRSLRSDDTAVYYCAR |
| | IGDSSPSDYWGQGILVTVSSGSTSGGGSGGGSGGGGSSEIVMTQSPAIL |
| | SVSPGERATLSCKASQSVSNDVVWYQQKPGQAPRLLIYDASNRNTGIPA |
| | RFSGSGSGTEFTLTISSLQSEDFAVYYCQQDYTSPWTFGQGTKLEIKEP |
| | KSSDKTHTCPPCPPCGGGSSGGGSGGQPREPQVYTLPPSREEMTKNQVS |
| | LTCLVKGFYPSDIAVEWESNGQPENNYKTTPPVLDSDGSFFLYSKLIVD |
| | KSRWQQGNVFSCSVMHEALHNHYTQKSLSLSPG |
| | (SEQ ID NO: 8) |
| IAB57M1-13 | METDTLLLWVLLLWVPGSTG |
| | EIVMTQSPATLSVSPGERATLSCKASQSVSNDVVWYQQKPGQAPQLLIY |
| | DASNRYTGIPARFSGSGSGTEFTLTISSLQSEDFAVYYCQQDYTSPWTF |
| | GQGTKLEIKGSTSGGGSGGGSGGGGSSQVQLVQSGAEVKKPGASVKVSC |
| | KASGYTFTNYGMNWVRQAPGQGLEWMGWINTYTGEPTYADDFKGRVTMT |
| | TDTSTSTAYMELRSLRSDDTAVYYCARIGDSSPSDYWGQGTLVTVSSEP |
| | KSSDKTHTCPPCPPCGGGSSGGGSGGQPREPQVYTLPPSREEMTKNQVS |
| | LTCLVKGFYPSDIAVEWESNGQPENNYKITPPVLDSDGSFFLYSKLIVD |
| | KSRWQQGNVFSCSVMHEALHNHYTQKSLSLSPG |
| | (SEQ ID NO: 9) |
| IAB57M1-14 | METDILLLWVLLLWVPGSTG |
| | EIVMTQSPATLSVSPGERATLSCKASQSVSNDVVWYQQKPGQSPQLLIY |
| | DASNRYTGIPARFSGSGSGTEFTLTISSLQSEDFAVYYCQQDYTSPWTF |
| | GQGTKLEIKGSTSGGGSGGGSGGGGSSQVQLVQSGAEVKKPGASVKVSC |
| | KASGYTFTNYGMNWVRQAPGQGLEWMGWINTYTGEPTYADDFKGRVTMT |
| | TDTSTSTAYMELRSLRSDDTAVYYCARIGDSSPSDYWGQGTLVTVSSEP |
| | KSSDKTHTCPPCPPCGGGSSGGGSGGQPREPQVYTLPPSREEMTKNQVS |
| | LTCLVKGFYPSDIAVEWESNGQPENNYKITPPVLDSDGSFFLYSKLTVD |
| | KSRWQQGNVFSCSVMHEALHNHYTQKSLSLSPG |
| | (SEQ ID NO: 10) |

In some embodiments, any of the constructs provided herein can be employed without or can exclude the signal peptide (e.g., the initial sequence underlined in Table 5 or 6). In some embodiments, the signal sequence is SEQ ID NO: 45. In some embodiments, the signal sequence is located immediately upstream of the antigen binding construct, minibody, or cys-diabody. In some embodiments, residue 20 of the signal peptide as numbered in FIG. 3 (Kabat) is immediately adjacent to residue number 1 of the antigen binding construct, minibody, and/or cys-diahody, as numbered in FIG. 1 (Kabat). In any and every embodiment provided herein, the signal sequence is optional, and can be included or excluded from any of the herein disclosed antigen binding construct, minibody, and/or cys-diabody. In some embodiments, the target molecule is detected using one or more of the cys-diabodies arrangements provided in the figures and/or in the Tables, such as Table 6.

TABLE 6

| Name | Sequence |
|---|---|
| IAB57C1-9 | METDTLLLWVLLLWVPGSTG |
| | EIVMTQSPATLSVSPGERATLSCKASQSVSNDVVWYQQKPGQAPRLLIY |
| | DASNRYTGIPARFSGSGSGTEFTLTISSLQSEDFAVYYCQQDYTSPWTF |
| | GQGTKLEIKSGGGGQVQLVQSGAEVKKPGASVKVSCKASGYTFTNYGMN |
| | WVRQAPGQGLEWMGWINTYTGEPTYADDFKGRVTMTTDTSTSTAYMELR |
| | SLRSDDTAVYYCARIGDSSPSDYWGQGTLVTVSSGGCPPCPPC |
| | (SEQ ID NO: 11) |
| IAB57C2-10 | METDILLLWVLLLWVPGSTG |
| | QVQLVQSGAEVKKPGASVKVSCKASGYTFTNYGMNWVRQAPGQGLEWMG |
| | WINTYTGEPTYADDFKGRVTMTTDTSTSTAYMELRSLRSDDTAVYYCAR |
| | IGDSSPSDYWGQGTLVTVSSSGGGGEIVMTQSPATLSVSPGERATLSCK |
| | ASQSVSNDVVWYQQKPGQAPRLLIYDASNRYTGIPARFSGSGSGTEFTL |
| | TISSLQSEDFAVYYCQQDYTSPWTFGQGTKLEIKGGCPPCPPC |
| | (SEQ ID NO: 12) |
| IAB57C3-11 | METDTLLLWVLLLWVPGSTG |
| | EIVMTQSPATLSVSPGERATLSCKASQSVSNDVVWYQQKPGQAPRLLIY |
| | DASNRYTGIPARFSGSGSGTEFTLTISSLQSEDFAVYYCQQDYTSPWTF |
| | GQGTKLEIKGGGSGGGGQVQLVQSGAEVKKPGASVKVSCKASGYTFTNY |
| | GMNWVRQAPGQGLEWMGWINTYTGEPTYADDFKGRVTMTTDISTSTAYM |
| | ELRSLRSDDTAVYYCARIGDSSPSDYWGQGTLVTVSSGGCPPCPPC |
| | (SEQ ID NO: 13) |
| IAB57C4-12 | METDTLLLWVLLLWVPGSTG |
| | QVQLVQSGAEVKKPGASVKVSCKASGYTFTNYGMNWVRQAPGQGLEWMG |
| | WINTYTGEPTYADDFKGRVTMTTDISTSTAYMELRSLRSDDTAVYYCAR |
| | IGDSSPSDYWGQGTLVTVSSGGGSGGGGEIVMTQSPATLSVSPGERATL |
| | SCKASQSVSNDVVWYQQKPGQAPRLLIYDASNRYTGIPARFSGSGSGTE |
| | FTLTISSLQSEDFAVYYCQQDYTSPWTFGQGTKLEIKGGCPPCPPC |
| | (SEQ ID NO: 14) |

Methods of detecting the presence or absence of the target molecule are provided herein. It will be appreciated that the processes below can be performed in any sequence, and/or can be optionally repeated and/or eliminated, and that additional steps can optionally be added to the method. In some embodiments, an antigen binding construct, minibody, or cys-diabody, as described herein can be applied to a sample. In some embodiments, an optional wash can be performed. Optionally, a secondary antigen binding construct can be applied to the sample. An optional wash can be performed. In some embodiments, a binding or absence of binding of the antigen binding construct, minibody, or cys-diabody, to the target molecule can be detected.

In some embodiments, an antigen binding construct, minibody, or cys-diabody, as described herein is applied to a sample in vivo. The antigen binding construct can be administered to a subject. In some embodiments, the subject is a human. In some embodiments, the subject is a non-human mammal, for example a rat, mouse, guinea pig, hamster, rabbit, dog, cat, cow, horse, goat, sheep, donkey, pig, monkey, or ape. In some embodiments, the antigen binding construct is infused into the subject. In some embodiments, the infusion is intravenous. In some embodiments, the infusion is intraperitoneal. In some embodiments, the antigen binding construct, minibody, or cys-diabody, is applied topically or locally (as in the case of an interventional or intraoperative application) to the subject. In some embodiments, a capsule containing the antigen binding construct, minibody, or cys-diabody, is applied to the subject, for example orally or intraperitoneally. In some embodiments, the antigen binding construct, minibody, or cys-diabody, is selected to reduce the risk of an immunogenic response by subject. For example, for a human subject, the antigen binding construct, minibody, or cys-diabody, can be humanized as described herein. In some embodiments, following in vivo application of the antigen binding construct, minibody, or cys-diabody, the sample, or a portion of the sample is removed from the host. In some embodiments, the antigen binding construct, minibody, or cys-diabody, is applied in vivo, is incubated in vivo for a period of time as described herein, and a sample is removed for analysis in vitro, for example in vitro detection of antigen binding construct, minibody, or cys-diabody, bound to the target molecule or the absence thereof as described herein.

In some embodiments, the antigen binding construct, minibody, or cys-diabody, is applied to a sample in vitro. In some embodiments, the sample is freshly harvested from a subject, for example a biopsy. In some embodiments, the sample is incubated following harvesting from a subject. In some embodiments, the sample is fixed. In some embodiments the sample includes a whole organ and/or tissue. In some embodiments, the sample includes one or more whole cells. In some embodiments the sample is from cell extracts, for example lysates. In some embodiments, antigen binding construct in solution is added to a solution in the sample. In some embodiments, antigen binding construct, minibody, or cys-diabody, in solution is added to a sample that does not contain a solution, for example a lyophilized sample, thus reconstituting the sample. In some embodiments, lyophilized antigen binding construct, minibody, or cys-diabody, is added to a sample that contains solution, thus reconstituting the antigen binding construct, minibody, or cys-diabody.

In some embodiments, the antigen binding construct, minibody, or cys-diabody, is optionally incubated with the sample. The antigen binding construct, minibody, or cys-diabody, can be incubated for a period of no more than about 14 days, for example no more than about 14 days, 13, 12, 11, 10, 9, 8, 7, 6, 5, 4, 3, 2, or 1 day, or no more than about 23 hours, for example no more than about 23 hours, 22, 21, 20, 19, 18, 17, 16, 15, 14, 13, 12, 11, 10, 9, 8, 7, 6, 5, 4, 3, 2, 1, 0.75, 0.5, 0.25, or 0.1 hour, or is a range that is defined by any two of the preceding values. In some embodiments, the incubation is within a subject to which the antigen binding construct, minibody, or cys-diabody, was administered. In some embodiments, the incubation is within an incubator. In some embodiments, the incubator is maintained at a fixed temperature, for example about 21° C., room temperature, 25° C., 29° C., 34° C., 37° C., or 40° C.

In some embodiments, the antigen binding construct, minibody, or cys-diabody, that is not bound to the target is optionally removed from the sample. In some embodiments, the sample is washed. Washing a sample can include removing the solution that contains unbound antigen binding construct, minibody, or cys-diabody, and adding solution that does not contain antigen binding construct, minibody, or cys-diabody, for example buffer solution. In some embodiments, an in vitro sample is washed, for example by aspirating, pipetting, pumping, or draining solution that contains unbound antigen binding construct, minibody, or cys-diabody, and adding solution that does not contain antigen binding construct, minibody, or cys-diabody. In some embodiments, an in vivo sample is washed, for example by administering to the subject solution that does not contain antigen binding construct, minibody, or cys-diabody, or by washing a site of topical antigen binding construct administration. In some embodiments, the wash is performed at least two times, for example at least 2, 3, 4, 5, 6, 7, 8, 9, 10, 15, or 20 times. In some embodiments, following the wash or washes, at least about 50% of unbound antibody is removed from the sample, for example at least about 50%, 60%, 70%, 75%, 80%, 85%, 90%, 95%, 97%, 98%, 99% or greater.

In some embodiments, unbound antigen binding construct, minibody, or cys-diabody is eliminated from the sample. Following application of the antigen binding construct, minibody, or cys-diabody, to the sample, antigen binding construct bound to the target reaches an equilibrium with antigen binding construct unbound to the target, so that at some time after application of the antigen binding construct, the amount of antigen binding construct bound to the target does not substantially increase. After this time, at least part of the quantity of the antigen binding construct, minibody, or cys-diabody, that is unbound to the target can be eliminated. In some embodiments, unbound antigen binding construct, minibody, or cys-diabody, is eliminated by metabolic or other bodily processes of the subject to whom the antigen binding construct, minibody, or cys-diabody, was delivered. In some embodiments, unbound antigen binding construct, minibody, or cys-diabody, is eliminated by the addition of an agent that destroys or destabilized the unbound antigen binding construct, minibody, or cys-diabody, for example a protease or a neutralizing antibody. In some embodiments, 1 day after application of the antigen binding construct, minibody, or cys-diabody, at least about 30% of the antigen binding construct that was applied has been eliminated, for example at least about 30%, 40%, 60%, 70%, 75%, 80%, 85%, 90%, 95%, 97%, 98%, 99%, or 99.9%. In some embodiments, 2 days after application of the antigen binding construct, minibody, or cys-diabody, at least about 40% of the antigen binding construct, minibody, or cys-diabody, that was applied has been eliminated, for example at least about 40%, 50%, 60%, 70%, 75%, 80%, 85%, 90%, 95%, 97%, 98%, 99%, or 99.9%.

In some embodiments, the presence or absence of the target molecule is detected. The presence or absence of the target can be detected based on the presence or absence of the antigen binding construct in the sample. After removal and/or elimination of the antigen binding construct from the sample, for example by washing and/or metabolic elimination, remaining antigen binding construct in the sample can indicate the presence of the target, while an absence of the antigen binding construct in the sample can indicate the absence of the target.

In some embodiments, the antigen binding construct includes a detectable marker as described herein. Thus, the presence of the antigen binding construct can be inferred by detecting the detectable marker.

In some embodiments, a secondary antigen binding construct, minibody, or cys-diabody, is used to detect the antigen binding construct, minibody, or cys-diabody. The secondary antigen binding construct, minibody, or cys-diabody, can bind specifically to the antigen binding construct. For example, the secondary antigen binding construct, minibody, or cys-diabody, can include a polyclonal or monoclonal antibody, diabody, minibody, etc. against the host type of the antibody, or against the antigen binding construct, minibody, or cys-diabody, itself. The secondary antigen binding construct, minibody, or cys-diabody, can be conjugated to a detectable marker as described herein. The secondary antigen binding construct, minibody, or cys-diabody, can be applied to the sample. In some embodiments, the secondary antigen binding construct, minibody, or cys-diabody, is applied to the sample in substantially the same manner as the antigen binding construct, minibody, or cys-diabody. For example, if the antigen binding construct, minibody, or cys-diabody, was infused into a subject, the secondary antigen binding construct, minibody, or cys-diabody, can also be infused into the subject.

In some embodiments, binding or the absence of binding of the antigen binding construct, minibody, or cys-diabody, is detected via at least one of: positron emission tomography (PET), single-photon emission computed tomography (SPECT), magnetic resonance imaging (MRI), Computed Tomography (CT), or detection of fluorescence emissions. PET can include, but is not limited to small animal PET imaging. In some embodiments, binding of the absence of binding of the antigen binding construct, minibody, or cys-diabody is detected via two or more forms of imaging. In some embodiments, detection can be via near-infrared (NIR) and/or Cerenkov.

In some embodiments, any combination of imaging modalities is possible, including, by way of example, PET+CR, SPECT+CT, PET+MRI, PET+NIR, PET+SPECT etc. “PET” is a diagnostic technique that can be used to observe functions and metabolism of human organs and tissues at the molecular level. For PET, a positron radioactive drug (e.g., 18F-FDG) can be injected into a human body. If FDG is used, because the metabolism of fludeoxyglucose (FDG) is similar to glucose, the FDG will gather in cells that digest the glucose. A positron emitted by the decay of 18F and an electron in tissues will undergo an annihilation reaction to generate two gamma-photons with the same energy in opposite directions. A detector array surrounding the human body can detect the two photons using a coincidence measurement technique, and determine position information of the positron. A tomography image of positrons in the human body can then be constructed by processing the position information using an image reconstruction software. In some situations, Immuno-PET can be employed, where the label (e.g., 18F) is attached or associated with an antigen binding construct. In such embodiments, the distribution of the antigen binding construct can be monitored, which will depend upon the binding properties and distribution properties of the antigen binding construct. For example, if a CD8-directed minibody is used, then PET can be used to monitor the distribution of the CD8 molecules through the hosts' system. PET systems are known in the art and include, for example U.S. Pat. Pub. No. 20170357015, 20170153337, 20150196266, 20150087974, 20120318988, and 20090159804, the entireties of each of which are incorporated by reference herein for their description regarding PET and the use thereof.

In some embodiments, imaging is conducted through PET scan, a PET/CT scan, or SPECT scan. In some embodiments, imaging is conducted through photoacoustics, optical probes, MR imaging, magnetic nanoparticles for imaging, spectroscopy, and/or any other standard method of imaging. In some embodiments, at least one optical probe is coupled together with an at least one metal chelator. In some embodiments, optical imaging is used for assisted surgery. In some embodiments, photodynamic therapy is used. In some embodiments, photodynamic therapy is used for assisted surgery. In some embodiments, infrared fluorescence is used for assisted surgery. In some embodiments, photodynamic therapy is used for theranostics.

### Nucleic Acid

In some embodiments, the polypeptides of the antigen binding constructs, minibodies, or cys-diabodies, can be encoded by nucleic acids and expressed in vivo or in vitro, or these peptide can be synthesized chemically. Thus, in some embodiments, a nucleic acid encoding an antigen binding construct, minibody, or cys-diabody, is provided. In some embodiments, the nucleic acid encodes one part or monomer of an antigen binding construct, minibody, or cys-diabody. In some embodiments, the nucleic acid encodes two or more monomers, for example, at least 2 monomers. Nucleic acids encoding multiple monomers can include nucleic acid cleavage sites between at least two monomers, can encode transcription or translation start site between two or more monomers, and/or can encode proteolytic target sites between two or more monomers. In some embodiments, the nucleic acid encodes an antigen binding construct, minibody, cys-diabody, VL, VH, CDR, FR, linker, signal peptide, or any combination therein. In some embodiments, the nucleic acid encodes an antigen binding constructs, minibodies, cys-diabodies, VL, VH, CDR, FR, linker, signal peptide, or any combination therein, having at least about 70%, 75%, 80%, 85%, 90%, 95%, 96%, 97%, 98% or 99% sequence identity to an antigen binding construct, minibody, cys-diabody, VL, VH, CDR, FR, linker, signal peptide, of any of SEQ ID NO: 1-48. In some embodiments, the nucleic acid encodes a minibody that is any of SEQ ID NO: 1-10. In some embodiments, the nucleic acid encodes a minibody having at least about 70%, 75%, 80%, 85%, 90%, 95%, 96%, 97%, 98% or 99% sequence identity to a minibody that is any of SEQ ID NO: 1-10. In some embodiments, the minibody encoding nucleic acid is a nucleic acid having at least about 70%, 75%, 80%, 85%, 90%, 95%, 96%, 97%, 98% or 99% sequence identity to a nucleic acid that is any of SEQ ID NO: 51-58, or 64-65. In some embodiments, the nucleic acid encodes a cys-diabody that is any of SEQ ID NO: 11-14. In some embodiments, the nucleic acid encodes a cys-diabody having at least about 70%, 75%, 80%, 85%, 90%, 95%, 96%, 97%, 98% or 99% sequence identity to a cys-diabody that is any of SEQ ID NO: 11-14. In some embodiments, the cys-diabody encoding nucleic acid is a nucleic acid having at least about 70%, 75%, 80%, 85%, 90%, 95%, 96%, 97%, 98% or 99% sequence identity to a nucleic acid that is any of SEQ ID NO: 59-63. In some embodiments, the nucleic acid encodes a LCDR that is any of SEQ ID NO: 15-26. In some embodiments, the nucleic acid encodes a LCDR having at least about 70%, 75%, 80%, 85%, 90%, 95%, 96%, 97%, 98% or 99% sequence identity to a LCDR that is any of SEQ ID NO: 15-26. In some embodiments, the nucleic acid encodes a HCDR that is any of SEQ ID NO: 27-32. In some embodiments, the nucleic acid encodes a HCDR having at least about 70%, 75%, 80%, 85%, 90%, 95%, 96%, 97%, 98% or 99% sequence identity to a HCDR that is any of SEQ ID NO: 27-32. In some embodiments, the nucleic acid encodes a FR that is any of SEQ ID NO: 33-38. In some embodiments, the nucleic acid encodes a FR having at least about 70%, 75%, 80%, 85%, 90%, 95%, 96%, 97%, 98% or 99% sequence identity to a FR that is any of SEQ ID NO: 33-38. In some embodiments, the nucleic acid encodes a linker that is any of SEQ ID NO: 39-44. In some embodiments, the nucleic acid encodes a linker having at least about 70%, 75%, 80%, 85%, 90%, 95%, 96%, 97%, 98% or 99% sequence identity to a linker that is any of SEQ ID NO: 39-44. In some embodiments, the nucleic acid encodes a signal peptide that is any of SEQ ID NO: 45-46. In some embodiments, the nucleic acid encodes a signal peptide having at least about 70%, 75%, 80%, 85%, 90%, 95%, 96%, 97%, 98% or 99% sequence identity to a signal peptide that is any of SEQ ID NO: 45-46. In some embodiments, the nucleic acid encodes a VH that is SEQ ID NO: 47. In some embodiments, the nucleic acid encodes a minibody having at least about 70%, 75%, 80%, 85%, 90%, 95%, 96%, 97%, 98% or 99% sequence identity to a VH that is SEQ ID NO: 47. In some embodiments, the nucleic acid encodes a VL that is SEQ ID NO: 48. In some embodiments, the nucleic acid encodes a VL having at least about 70%, 75%, 80%, 85%, 90%, 95%, 96%, 97%, 98% or 99% sequence identity to a VL that is any of SEQ ID NO: 48.

In some embodiments, an expression vector contains a nucleic acid encoding an antigen binding construct, minibody, or cys-diabody, as disclosed herein. In some embodiments, the expression vector includes pcDNA3.1™/myc-His (−) Version A vector for mammalian expression (Invitrogen, Inc.) or a variant thereof. The pcDNA3.1 expression vector features a CMV promoter for mammalian expression and both mammalian (Neomycin) and bacterial (Ampicillin) selection markers. In some embodiments, the expression vector includes a plasmid. In some embodiments, the vector includes a viral vector, for example a retroviral or adenoviral vector. In embodiments, the vector includes a cosmid, YAC, or BAC. In some embodiments, the expression vector encodes an antigen binding construct, minibody, cys-diabody, VL, VH, CDR, FR, linker, signal peptide, or any combination therein. In some embodiments, the expression vector encodes an antigen binding construct, minibody, cys-diabody, VL, VH, CDR, FR, linker, signal peptide, or any combination therein, having at least about 70%, 75%, 80%, 85%, 90%, 95%, 96%, 97%, 98% or 99% sequence identity to an antigen binding construct, minibody, cys-diabody, VL, VH, CDR, FR, linker, signal peptide, of any of SEQ ID NO: 1-48. In some embodiments, the expression vector encodes a minibody that is any of SEQ ID NO: 1-10. In some embodiments, the expression vector encodes a minibody having at least about 70%, 75%, 80%, 85%, 90%, 95%, 96%, 97%, 98% or 99% sequence identity to a minibody that is any of SEQ ID NO: 1-10. In some embodiments, the expression vector encodes a cys-diabody that is any of SEQ ID NO: 11-14. In some embodiments, the expression vector encodes a cys-diabody having at least about 70%, 75%, 80%, 85%, 90%, 95%, 96%, 97%, 98% or 99% sequence identity to a cys-diabody that is any of SEQ ID NO: 11-14. In some embodiments, the expression vector encodes a LCDR that is any of SEQ ID NO: 15-26. In some embodiments, the expression vector encodes a LCDR having at least about 70%, 75%, 80%, 85%, 90%, 95%, 96%, 97%, 98% or 99% sequence identity to a LCDR that is any of SEQ ID NO: 15-26. In some embodiments, the expression vector encodes a HCDR that is any of SEQ ID NO: 27-32. In some embodiments, the expression vector encodes a HCDR having at least about 70%, 75%, 80%, 85%, 90%, 95%, 96%, 97%, 98% or 99% sequence identity to a HCDR that is any of SEQ ID NO: 27-32. In some embodiments, the expression vector encodes a FR that is any of SEQ ID NO: 33-38. In some embodiments, the expression vector encodes a FR having at least about 70%, 75%, 80%, 85%, 90%, 95%, 96%, 97%, 98% or 99% sequence identity to a FR that is any of SEQ ID NO: 33-38. In some embodiments, the expression vector encodes a linker that is any of SEQ ID NO: 39-44. In some embodiments, the expression vector encodes a linker having at least about 70%, 75%, 80%, 85%, 90%, 95%, 96%, 97%, 98% or 99% sequence identity to a linker that is any of SEQ ID NO: 39-44. In some embodiments, the expression vector encodes a signal peptide that is any of SEQ ID NO: 45-46. In some embodiments, the expression vector encodes a signal peptide having at least about 70%, 75%, 80%, 85%, 90%, 95%, 96%, 97%, 98% or 99% sequence identity to a signal peptide that is any of SEQ ID NO: 45-46. In some embodiments, the expression vector encodes a VH that is SEQ ID NO: 47. In some embodiments, the nucleic acid encodes a minibody having at least about 70%, 75%, 80%, 85%, 90%, 95%, 96%, 97%, 98% or 99% sequence identity to a VH that is SEQ ID NO: 47. In some embodiments, the expression vector encodes a VL that is SEQ ID NO: 48. In some embodiments, the expression vector encodes a VL having at least about 70%, 75%, 80%, 85%, 90%, 95%, 96%, 97%, 98% or 99% sequence identity to a VL that is any of SEQ ID NO: 48. In any of the embodiments provided herein, the signal peptide can be excluded from the amino acid sequence, for some embodiments.

FIG. 14 shows some embodiments of a nucleic acid sequence of antigen binding constructs, minibodies, and/or cys-diabodies. (SEQ ID NO: 51-64). In some embodiments the nucleic acid binding construct and/or minibody is an antigen binding construct and/or minibody in Table 6. In some embodiments, the expression vector encodes an antigen binding construct and/or minibody, that is an antigen binding construct and/or minibody in Table 7.

TABLE 7

| Name | Sequence |
|---|---|
| IAB57M1-1 | ATGGAGACCGACACCCTGCTGCTGTGGGTGCTCCTACTGTGGGTGCCCGGCAG |
| | CACCGGCGAGATCGTGATGACACAGAGCCCCGCGACACTCAGCGTGAGTCCCG |
| | GCGAGAGAGCGACGCTCAGCTGCAAGGCAAGCCAAAGCGTGAGCAACGACGTG |
| | GTGTGGTATCAGCAGAAGCCCGGCCAAGCCCCTAGACTGCTGATCTACTACGC |
| | TAGCAACAGATACACCGGCATCCCCGCTAGATTCAGCGGCAGCGGCAGCGGCA |
| | CCGAGTTCACCCTGACCATCAGCAGCCTGCAGAGCGAGGACTTCGCCGTTTAC |
| | TACTGTCAACAAGACTACACAAGCCCCTGGACCTTCGGCCAAGGCACCAAGCT |
| | GGAAATTAAGGGCAGCACCTCTGGAGGCGGGTCGGGCGGAGGATCGGGAGGCG |
| | GCGGAAGCAGCCAAGTGCAGCTGGTGCAGAGCGGCGCCGAGGTGAAGAAGCCC |
| | GGCGCTAGCGTGAAGGTATCTTGCAAGGCTAGCGGGTATACCTTCACCAACTA |
| | CGGCATGAACTGGGTGAGACAAGCCCCCGGCCAAGGCCTGGAGTGGATGGGCT |
| | GGATCAACACCTACACCGGCGAGCCCACCTACGCCGACGACTTCAAGGGCAGA |
| | GTGACCATGACCACCGACACAAGCACAAGCACCGCCTACATGGAGCTGAGAAG |
| | CCTGAGAAGCGACGACACCGCTGTGTATTATTGCGCGCGGATCGGCGACAGCA |
| | GCCCTAGCGACTACTGGGGACAAGGCACCCTGGTTACCGTGAGCAGCGAACCC |
| | AAGAGCTCCGACAAGACCCACACgTGTCCCCCTTGCCCTCCTTGTGGCGGAGG |
| | AAGCTCCGGAGGCGGAAGCGGAGGACAGCCTAGGGAGCCCCAGGTGTATACCC |
| | TCCCCCCCTCCAGGGAAGAGATGACCAAGAACCAGGTGAGCCTGACCTGCCTC |
| | GTGAAGGGCTTTTATCCCTCCGATATCGCCGTGGAGTGGGAGAGCAACGGCCA |
| | GCCTGAGAACAATTACAAGACCACCCCCCCTGTGCTGGACTCCGATGGCAGCT |
| | TCTTCCTGTATTCCAAGCTGACCGTCGACAAGTCCAGGTGGCAACAGGGCAAC |
| | GTCTTCAGCTGCAGCGTGATGCACGAGGCCCTGCACAATCACTACACCCAGAA |
| | GTCCCTCTCCCTGAGCCCCGGCTGA |
| | (SEQ ID NO: 51) |
| IAB57M2-2 | ATGGAGACCGATACCCTGCTACTGTGGGTCCTCCTACTGTGGGTGCCCGGCAG |
| | CACCGGGCAAGTGCAGCTGGTGCAGAGCGGCGCCGAGGTGAAGAAGCCCGGCG |
| | CTAGCGTGAAGGTGAGCTGCAAGGCAAGCGGTTACACCTTCACCAACTACGGC |
| | ATGAACTGGGTGAGACAAGCCCCCGGCCAAGGCCTGGAGTGGATGGGCTGGAT |
| | CAACACCTACACCGGCGAGCCCACCTACGCCGACGACTTCAAGGGCAGAGTGA |
| | CCATGACCACCGACACAAGCACAAGCACCGCCTACATGGAGCTGAGAAGCCTG |
| | AGAAGCGACGACACCGCCGTTTACTACTGTGCGAGAATCGGCGACAGCAGCCC |
| | TAGCGACTATTGGGGTCAAGGCACCTTGGTTACTGTGTCAAGCGGCTCCACCT |
| | CCGGCGGCGGATCGGGCGGAGGAAGCGGTGGCGGCGGCAGCAGCGAGATCGTG |
| | ATGACACAGAGCCCCGCCACCTTAAGCGTTAGCCCCGGCGAGAGAGCAACCCT |
| | TTCTTGCAAAGCTAGCCAAAGTGTGAGCAACGACGTGGTGTGGTATCAGCAGA |
| | AGCCCGGCCAAGCCCCTAGACTGCTGATCTACTACGCTAGCAACAGATACACC |
| | CCCATCCCCCCTACATTCACCCCCACCGGCAGCCCCACCGAGTTCACCCTCAC |
| | CATCAGCAGCCTGCAGAGCGAGGACTTCGCTGTGTATTATTGCCAACAAGACT |
| | ACACAAGCCCCTGGACCTTCGGTCAAGGGACCAAGCTGGAGATCAAGGAACCC |
| | AAGAGCTCCGACAAGACCCACACGTGTCCCCCTTGCCCTCCTTGTGGCGGAGG |
| | AAGCTCCGGAGGCGGAAGCGGAGGACAGCCTAGGGAGCCCCAGGTGTATACCC |
| | TCCCCCCCTCCAGGGAAGAGATGACCAAGAACCAGGTGAGCCTGACCTGCCTC |
| | GTGAAGGGCTTTTATCCCTCCGATATCGCCGTGGAGTGGGAGAGCAACGGCCA |
| | GCCTGAGAACAATTACAAGACCACCCCCCCTGTGCTGGACTCCGATGGCAGCT |
| | TCTTCCTGTATTCCAAGCTGACCGTCGACAAGTCCAGGTGGCAACAGGGCAAC |
| | GTCTTCAGCTGCAGCGTGATGCACGAGGCCCTGCACAATCACTACACCCAGAA |
| | GTCCCTCTCCCTGAGCCCCGGCTGA |
| | (SEQ ID NO: 52) |
| IAB57M1-3 | ATGGAGACCGACACCCTGCTGCTGTGGGTGCTCCTACTGTGGGTGCCCGGCAG |
| | CACCGGCGAGATCGTGATGACACAGAGCCCCGCGACACTCAGCGTGAGTCCCG |
| | GCGAGAGAGCGACGCTCAGCTGCAAGGCAAGCCAAAGCGTGAGCAACGACGTG |
| | GTGTGGTATCAGCAGAAGCCCGGCCAAGCCCCTAGACTGCTGATCTACGACGC |
| | TAGCAACAGATACACCGGCATCCCCGCTAGATTCAGCGGCAGCGGCAGCGGCA |
| | CCGAGTTCACCCTGACCATCAGCAGCCTGCAGAGCGAGGACTTCGCCGTTTAC |
| | TACTGTCAACAAGACTACACAAGCCCCTGGACCTTCGGCCAAGGCACCAAGCT |
| | GGAAATTAAGGGCAGCACCTCTGGAGGCGGGTCGGGCGGAGGATCGGGAGGCG |
| | GCGGAAGCAGCCAAGTGCAGCTGGTGCAGAGCGGCGCCGAGGTGAAGAAGCCC |
| | GGCGCTAGCGTGAAGGTATCTTGCAAGGCTAGCGGGTATACCTTCACCAACTA |
| | CGGCATGAACTGGGTGAGACAAGCCCCCGGCCAAGGCCTGGAGTGGATGGGCT |
| | GGATCAACACCTACACCGGCGAGCCCACCTACGCCGACGACTTCAAGGGCAGA |
| | GTGACCATGACCACCGACACAAGCACAAGCACCGCCTACATGGAGCTGAGAAG |
| | CCTGAGAAGCGACGACACCGCTGTGTATTATTGCGCGCGGATCGGCGACAGCA |
| | GCCCTAGCGACTACTGGGGACAAGGCACCCTGGTTACCGTGAGCAGCGAACCC |
| | AAGAGCTCCGACAAGACCCACACgTGTCCCCCTTGCCCTCCTTGTGGCGGAGG |
| | AAGCTCCGGAGGCGGAAGCGGAGGACAGCCTAGGGAGCCCCAGGTGTATACCC |
| | TCCCCCCCTCCAGGGAAGAGATGACCAAGAACCAGGTGAGCCTGACCTGCCTC |
| | GTGAAGGGCTTTTATCCCTCCGATATCGCCGTGGAGTGGGAGAGCAACGGCCA |
| | GCCTGAGAACAATTACAAGACCACCCCCCCTGTGCTGGACTCCGATGGCAGCT |
| | TCTTCCTGTATTCCAAGCTGACCGTCGACAAGTCCAGGTGGCAACAGGGCAAC |
| | GTCTTCAGCTGCAGCGTGATGCACGAGGCCCTGCACAATCACTACACCCAGAA |
| | GTCCCTCTCCCTGAGCCCCGGCTGA |
| | (SEQ ID NO: 53) |
| IAB57M2-4 | ATGGAGACCGATACCCTGCTACTGTGGGTCCTCCTACTGTGGGTGCCCGGCAG |
| | CACCGGGCAAGTGCAGCTGGTGCAGAGCGGCGCCGAGGTGAAGAAGCCCGGCG |
| | CTAGCGTGAAGGTGAGCTGCAAGGCAAGCGGTTACACCTTCACCAACTACGGC |
| | ATGAACTGGGTGAGACAAGCCCCCGGCCAAGGCCTGGAGTGGATGGGCTGGAT |
| | CAACACCTACACCGGCGAGCCCACCTACGCCGACGACTTCAAGGGCAGAGTGA |
| | CCATGACCACCGACACAAGCACAAGCACCGCCTACATGGAGCTGAGAAGCCTG |
| | AGAAGCGACGACACCGCCGTTTACTACTGTGCGAGAATCGGCGACAGCAGCCC |
| | TAGCGACTATTGGGGTCAAGGCACCTTGGTTACTGTGTCAAGCGGCTCCACCT |
| | CCGGCGGCGGATCGGGCGGAGGAAGCGGTGGCGGCGGCAGCAGCGAGATCGTG |
| | ATGACACAGAGCCCCGCCACCTTAAGCGTTAGCCCCGGCGAGAGAGCAACCCT |
| | TTCTTGCAAAGCTAGCCAAAGTGTGAGCAACGACGTGGTGTGGTATCAGCAGA |
| | AGCCCGGCCAAGCCCCTAGACTGCTGATCTACGACGCTAGCAACAGATACACC |
| | GGCATCCCCGCTAGATTCAGCGGCAGCGGCAGCGGCACCGAGTTCACCCTGAC |
| | CATCAGCAGCCTGCAGAGCGAGGACTTCGCTGTGTATTATTGCCAACAAGACT |
| | ACACAAGCCCCTCGACCTTCGGTCAAGGGACCAAGCTGGAGATCAAGGAACCC |
| | AAGAGCTCCGACAAGACCCACACgTGTCCCCCTTGCCCTCCTTGTGGCGGAGG |
| | AAGCTCCGGAGGCGGAAGCGGAGGACAGCCTAGGGAGCCCCAGGTGTATACCC |
| | TCCCCCCCTCCAGGGAAGAGATGACCAAGAACCAGGTGAGCCTGACCTGCCTC |
| | GTGAAGGGCTTTTATCCCTCCGATATCGCCGTGGAGTGGGAGAGCAACGGCCA |
| | GCCTGAGAACAATTACAAGACCACCCCCCCTGTGCTGGACTCCGATGGCAGCT |
| | TCTTCCTGTATTCCAAGCTGACCGTCGACAAGTCCAGGTGGCAACAGGGCAAC |
| | GTCTTCAGCTGCAGCGTGATGCACGAGGCCCTGCACAATCACTACACCCAGAA |
| | GTCCCTCTCCCTGAGCCCCGGCTGA |
| | (SEQ ID NO: 54) |
| IAB57M1-5 | ATGGAGACCGACACCCTGCTGCTGTGGGTGCTCCTACTGTGGGTGCCCGGCAG |
| | CACCGGCGAGATCGTGATGACACAGAGCCCCGCGACACTCAGCGTGAGTCCCG |
| | GCGAGAGAGCGACGCTCAGCTGCAAGGCAAGCCAAAGCGTGAGCAACGACGTG |
| | GTGTGGTATCAGCAGAAGCCCGGCCAAGCCCCTAGACTGCTGATCTACTACGC |
| | TAGCAACAGAAACACCGGCATCCCCGCTAGATTCAGCGGCAGCGGCAGCGGCA |
| | CCGAGTTCACCCTGACCATCAGCAGCCTGCAGAGCGAGGACTTCGCCGTTTAC |
| | TACTGTCAACAAGACTACACAAGCCCCTGGACCTTCGGCCAAGGCACCAAGCT |
| | GGAAATTAAGGGCAGCACCTCTGGAGGCGGGTCGGGCGGAGGATCGGGAGGCG |
| | GCGGAAGCAGCCAAGTGCAGCTGGTGCAGAGCGGCGCCGAGGTGAAGAAGCCC |
| | GGCGCTAGCGTGAAGGTATCTTGCAAGGCTAGCGGGTATACCTTCACCAACTA |
| | CGGCATGAACTGGGTGAGACAAGCCCCCGGCCAAGGCCTGGAGTGGATGGGCT |
| | GGATCAACACCTACACCGGCGAGCCCACCTACGCCGACGACTTCAAGGGCAGA |
| | GTGACCATGACCACCGACACAAGCACAAGCACCGCCTACATGGAGCTGAGAAG |
| | CCTGAGAAGCGACGACACCGCTGTGTATTATTGCGCGCGGATCGGCGACAGCA |
| | GCCCTAGCGACTACTGGGGACAAGGCACCCTGGTTACCGTGAGCAGCGAACCC |
| | AAGAGCTCCGACAAGACCCACACGTGTCCCCCTTGCCCTCCTTGTGGCGGAGG |
| | AAGCTCCGGAGGCGGAAGCGGAGGACAGCCTAGGGAGCCCCAGGTGTATACCC |
| | TCCCCCCCTCCAGGGAAGAGATGACCAAGAACCAGGTGAGCCTGACCTGCCTC |
| | GTGAAGGGCTTTTATCCCTCCGATATCGCCGTGGAGTGGGAGAGCAACGGCCA |
| | GCCTGAGAACAATTACAAGACCACCCCCCCTGTGCTGGACTCCGATGGCAGCT |
| | TCTTCCTGTATTCCAAGCTGACCGTCGACAAGTCCAGGTGGCAACAGGGCAAC |
| | GTCTTCAGCTGCAGCGTGATGCACGAGGCCCTGCACAATCACTACACCCAGAA |
| | GTCCCTCTCCCTGAGCCCCGGCTGA |
| | (SEQ ID NO: 55) |
| IAB57M2-6 | ATGGAGACCGATACCCTGCTACTGTGGGTCCTCCTACTGTGGGTGCCCGGCAG |
| | CACCGGGCAAGTGCAGCTGGTGCAGAGCGGCGCCGAGGTGAAGAAGCCCGGCG |
| | CTAGCGTGAAGGTGAGCTGCAAGGCAAGCGGTTACACCTTCACCAACTACGGC |
| | ATGAACTGGGTGAGACAAGCCCCCGGCCAAGGCCTGGAGTGGATGGGCTGGAT |
| | CAACACCTACACCGGCGAGCCCACCTACGCCGACGACTTCAAGGGCAGAGTGA |
| | CCATGACCACCGACACAAGCACAAGCACCGCCTACATGGAGCTGAGAAGCCTG |
| | AGAAGCGACGACACCGCCGTTTACTACTGTGCGAGAATCGGCGACAGCAGCCC |
| | TAGCGACTATTGGGGTCAAGGCACCTTGGTTACTGTGTCAAGCGGCTCCACCT |
| | CCGGCGGCGGATCGGGCGGAGGAAGCGGTGGCGGCGGCAGCAGCGAGATCGTG |
| | ATGACACAGAGCCCCGCCACCTTAAGCGTTAGCCCCGGCGAGAGAGCAACCCT |
| | TTCTTGCAAAGCTAGCCAAAGTGTGAGCAACGACGTGGTGTGGTATCAGCAGA |
| | AGCCCGGCCAAGCCCCTAGACTGCTGATCTACTACGCTAGCAACAGAAACACC |
| | GGCATCCCCGCTAGATTCAGCGGCAGCGGCAGCGGCACCGAGTTCACCCTGAC |
| | CATCAGCAGCCTGCAGAGCGAGGACTTCGCTGTGTATTATTGCCAACAAGACT |
| | ACACAAGCCCCTGGACCTTCGGTCAAGGGACCAAGCTGGAGATCAAGGAACCC |
| | AAGAGCTCCGACAAGACCCACACgTGTCCCCCTTGCCCTCCTTGTGGCGGAGG |
| | AAGCTCCGGAGGCGGAAGCGGAGGACAGCCTAGGGAGCCCCAGGTGTATACCC |
| | TCCCCCCCTCCAGGGAAGAGATGACCAAGAACCAGGTGAGCCTGACCTGCCTC |
| | GTGAAGGGCTTTTATCCCTCCGATATCGCCGTGGAGTGGGAGAGCAACGGCCA |
| | GCCTGAGAACAATTACAAGACCACCCCCCCTGTGCTGGACTCCGATGGCAGCT |
| | TCTTCCTGTATTCCAAGCTGACCGTCGACAAGTCCAGGTGGCAACAGGGCAAC |
| | GTCTTCAGCTGCAGCGTGATGCACGAGGCCCTGCACAATCACTACACCCAGAA |
| | GTCCCTCTCCCTGAGCCCCGGCTGA |
| | (SEQ ID NO: 56) |
| IAB57M1-7 | ATGGAGACCGACACCCTGCTGCTGTGGGTGCTCCTACTGTGGGTGCCCGGCAG |
| | CACCGGCGAGATCGTGATGACACAGAGCCCCGCGACACTCAGCGTGAGTCCCG |
| | GCGAGAGAGCGACGCTCAGCTGCAAGGCAAGCCAAAGCGTGAGCAACGACGTG |
| | GTGTGGTATCAGCAGAAGCCCGGCCAAGCCCCTAGACTGCTGATCTACGACGC |
| | TAGCAACAGAAACACCGGCATCCCCGCTAGATTCAGCGGCAGCGGCAGCGGCA |
| | CCGAGTTCACCCTGACCATCAGCAGCCTGCAGAGCGAGGACTTCGCCGTTTAC |
| | TACTGTCAACAAGACTACACAAGCCCCTGGACCTTCGGCCAAGGCACCAAGCT |
| | GGAAATTAAGGGCAGCACCTCTGGAGGCGGGTCGGGCGGAGGATCGGGAGGCG |
| | GCGGAAGCAGCCAAGTGCAGCTGGTGCAGAGCGGCGCCGAGGTGAAGAAGCCC |
| | GGCGCTAGCGTGAAGGTATCTTGCAAGGCTAGCGGGTATACCTTCACCAACTA |
| | CGGCATGAACTGGGTGAGACAAGCCCCCGGCCAAGGCCTGGAGTGGATGGGCT |
| | GGATCAACACCTACACCGGCGAGCCCACCTACGCCGACGACTTCAAGGGCAGA |
| | GTGACCATGACCACCGACACAAGCACAAGCACCGCCTACATGGAGCTGAGAAG |
| | CCTGAGAAGCGACGACACCGCTGTGTATTATTGCGCGCGGATCGGCGACAGCA |
| | GCCCTAGCGACTACTGGGGACAAGGCACCCTGGTTACCGTGAGCAGCGAACCC |
| | AAGAGCTCCGACAAGACCCACACGTGTCCCCCTTGCCCTCCTTGTGGCGGAGG |
| | AAGCTCCGGAGGCGGAAGCGGAGGACAGCCTAGGGAGCCCCAGGTGTATACCC |
| | TCCCCCCCTCCAGGGAAGAGATGACCAAGAACCAGGTGAGCCTGACCTGCCTC |
| | GTGAAGGGCTTTTATCCCTCCGATATCGCCGTGGAGTGGGAGAGCAACGGCCA |
| | GCCTGAGAACAATTACAAGACCACCCCCCCTGTGCTGGACTCCGATGGCAGCT |
| | TCTTCCTGTATTCCAAGCTGACCGTCGACAAGTCCAGGTGGCAACAGGGCAAC |
| | GTCTTCAGCTGCAGCGTGATGCACGAGGCCCTGCACAATCACTACACCCAGAA |
| | GTCCCTCTCCCTGAGCCCCGGCTGA |
| | (SEQ ID NO: 57) |
| IAB57M2-8 | TGGTGCAGAGCGGCGCCGAGGTGAAGAAGCCCGGCGCTAGCGTGAAGGTGAGC |
| | TGCAAGGCAAGCGGTTACACCTTCACCAACTACGGCATGAACTGGGTGAGACA |
| | AGCCCCCGGCCAAGGCCTGGAGTGGATGGGCTGGATCAACACCTACACCGGCG |
| | AGCCCACCTACGCCGACGACTTCAAGGGCAGAGTGACCATGACCACCGACACA |
| | AGCACAAGCACCGCCTACATGGAGCTGAGAAGCCTGAGAAGCGACGACACCGC |
| | CGTTTACTACTGTGCGAGAATCGGCGACAGCAGCCCTAGCGACTATTGGGGTC |
| | AAGGCACCTTGGTTACTGTGTCAAGCGGCTCCACCTCCGGCGGCGGATCGGGC |
| | GGAGGAAGCGGTGGCGGCGGCAGCAGCGAGATCGTGATGACACAGAGCCCCGC |
| | CACCTTAAGCGTTAGCCCCGGCGAGAGAGCAACCCTTTCTTGCAAAGCTAGCC |
| | AAAGTGTGAGCAACGACGTGGTGTGGTATCAGCAGAAGCCCGGCCAAGCCCCT |
| | AGACTGCTGATCTACGACGCTAGCAACAGAAACACCGGCATCCCCGCTAGATT |
| | CAGCGGCAGCGGCAGCGGCACCGAGTTCACCCTGACCATCAGCAGCCTGCAGA |
| | GCGAGGACTTCGCTGTGTATTATTGCCAACAAGACTACACAAGCCCCTGGACC |
| | TTCGGTCAAGGGACCAAGCTGGAGATCAAGGAACCCAAGAGCTCCGACAAGAC |
| | CCACACGTGTCCCCCTTGCCCTCCTTGTGGCGGAGGAAGCTCCGGAGGCGGAA |
| | GCGGAGGACAGCCTAGGGAGCCCCAGGTGTATACCCTCCCCCCCTCCAGGGAA |
| | GAGATGACCAAGAACCAGGTGAGCCTGACCTGCCTCGTGAAGGGCTTTTATCC |
| | CTCCGATATCGCCGTGGAGTGGGAGAGCAACGGCCAGCCTGAGAACAATTACA |
| | AGACCACCCCCCCTGTGCTGGACTCCGATGGCACCTTCTTCCTGTATTCCAAG |
| | CTGACCGTCGACAAGTCCAGGTGGCAACAGGGCAACGTCTTCAGCTGCAGCGT |
| | GATGCACGAGGCCCTGCACAATCACTACACCCAGAAGTCCCTCTCCCTGAGCC |
| | CCGGCTGA |
| | (SEQ ID NO: 58) |
| IAB57M1-13 | ATGGAGACCGACACCCTGCTGCTGTGGGTGCTCCTACTGTGGGTGCCCGGCAG |
| | CACCGGCGAGATCGTGATGACACAGAGCCCCGCGACACTCAGCGTGAGTCCCG |
| | CCCAGAGAGCGACGCTCAGCTGCAAGGCAAGCCAAAGCGTGAGCAACGACGTG |
| | GTGTGGTATCAGCAGAAGCCCGGCCAAGCCCCTCAACTGCTGATCTACGACGC |
| | TAGCAACAGATACACCGGCATCCCCGCTAGATTCAGCGGCAGCGGCAGCGGCA |
| | CCGAGTTCACCCTGACCATCAGCAGCCTGCAGAGCGAGGACTTCGCCGTTTAC |
| | TACTGTCAACAAGACTACACAAGCCCCTGGACCTTCGGCCAAGGCACCAAGCT |
| | GGAAATTAAGGGCAGCACCTCTGGAGGCGGGTCGGGCGGAGGATCGGGAGGCG |
| | GCGGAAGCAGCCAAGTGCAGCTGGTGCAGAGCGGCGCCGAGGTGAAGAAGCCC |
| | GGCGCTAGCGTGAAGGTATCTTGCAAGGCTAGCGGGTATACCTTCACCAACTA |
| | CGGCATGAACTGGGTGAGACAAGCCCCCGGCCAAGGCCTGGAGTGGATGGGCT |
| | GGATCAACACCTACACCGGCGAGCCCACCTACGCCGACGACTTCAAGGGCAGA |
| | GTGACCATGACCACCGACACAAGCACAAGCACCGCCTACATGGAGCTGAGAAG |
| | CCTGAGAAGCGACGACACCGCTGTGTATTATTGCGCGCGGATCGGCGACAGCA |
| | GCCCTAGCGACTACTGGGGACAAGGCACCCTGGTTACCGTGAGCAGCGAACCC |
| | AAGAGCTCCGACAAGACCCACACGTGTCCCCCTTGCCCTCCTTGTGGCGGAGG |
| | AAGCTCCGGAGGCGGAAGCGGAGGACAGCCTAGGGAGCCCCAGGTGTATACCC |
| | TCCCCCCCTCCAGGGAAGAGATGACCAAGAACCAGGTGAGCCTGACCTGCCTC |
| | GTGAAGGGCTTTTATCCCTCCGATATCGCCGTGGAGTGGGAGAGCAACGGCCA |
| | GCCTGAGAACAATTACAAGACCACCCCCCCTGTGCTGGACTCCGATGGCAGCT |
| | TCTTCCTGTATTCCAAGCTGACCGTCGACAAGTCCAGGTGGCAACAGGGCAAC |
| | GTCTTCAGCTGCAGCGTGATGCACGAGGCCCTGCACAATCACTACACCCAGAA |
| | GTCCCTCTCCCTGAGCCCCGGCTGA |
| | (SEQ ID NO: 63) |
| IAB57M1-14 | ATGGAGACCGACACCCTGCTGCTGTGGGTGCTCCTACTGTGGGTGCCCGGCAG |
| | CACCGGCGAGATCGTGATGACACAGAGCCCCGCGACACTCAGCGTGAGTCCCG |
| | GCGAGAGAGCGACGCTCAGCTGCAAGGCAAGCCAAAGCGTGAGCAACGACGTG |
| | GTGTGGTATCAGCAGAAGCCCGGCCAAAGCCCTCAACTGCTGATCTACGACGC |
| | TAGCAACAGATACACCGGCATCCCCGCTAGATTCAGCGGCAGCGGCAGCGGCA |
| | CCGAGTTCACCCTGACCATCAGCAGCCTGCAGAGCGAGGACTTCGCCGTTTAC |
| | TACTGTCAACAAGACTACACAAGCCCCTGGACCTTCGGCCAAGGCACCAAGCT |
| | GGAAATTAAGGGCAGCACCTCTGGAGGCGGGTCGGGCGGAGGATCGGGAGGCG |
| | GCGGAAGCAGCCAAGTGCAGCTGGTGCAGAGCGGCGCCGAGGTGAAGAAGCCC |
| | GGCGCTAGCGTGAAGGTATCTTGCAAGGCTAGCGGGTATACCTTCACCAACTA |
| | CGGCATGAACTGGGTGAGACAAGCCCCCGGCCAAGGCCTGGAGTGGATGGGCT |
| | GGATCAACACCTACACCGGCGAGCCCACCTACGCCGACGACTTCAAGGGCAGA |
| | GTGACCATGACCACCGACACAAGCACAAGCACCGCCTACATGGAGCTGAGAAG |
| | CCTGAGAAGCGACGACACCGCTGTGTATTATTGCGCGCGGATCGGCGACAGCA |
| | GCCCTAGCGACTACTGGGGACAAGGCACCCTGGTTACCGTGAGCAGCGAACCC |
| | AAGAGCTCCGACAAGACCCACACGTGTCCCCCTTGCCCTCCTTGTGGCGGAGG |
| | AAGCTCCGGAGGCGGAAGCGGAGGACAGCCTAGGGAGCCCCAGGTGTATACCC |
| | TCCCCCCCTCCAGGGAAGAGATGACCAAGAACCAGGTGAGCCTGACCTGCCTC |
| | GTGAAGGGCTTTTATCCCTCCGATATCGCCGTGGAGTGGGAGAGCAACGGCCA |
| | GCCTGAGAACAATTACAAGACCACCCCCCCTGTGCTGGACTCCGATGGCAGCT |
| | TCTTCCTGTATTCCAAGCTGACCGTCGACAAGTCCAGGTGGCAACAGGGCAAC |
| | GTCTTCAGCTGCAGCGTGATGCACGAGGCCCTGCACAATCACTACACCCAGAA |
| | GTCCCTCTCCCTGAGCCCCGGCTGA |
| | (SEQ ID NO: 64) |

In some embodiments the nucleic acid binding construct and/or minibody is an antigen binding construct and/or mninibody in Table 7. In some embodiments, the expression vector encodes an antigen binding construct and/or cys-diabody, that is an antigen binding construct and/or cys-diabody in Table 8.

TABLE 8

| Name | Sequence |
|---|---|
| IAB57C1-9 | ATGGAGACCGACACCCTGCTGCTGTGGGTGCTGCTCCTGTGGGTGCCCGGTAG |
| | CACCGGCGAGATCGTGATGACACAGAGCCCCGCGACCCTCAGCGTCAGTCCCG |
| | GCGAGAGAGCCACGCTGAGCTGCAAGGCATCTCAGAGCGTGAGCAACGACGTG |
| | GTGTGGTATCAGCAGAAGCCCGGCCAAGCCCCTAGACTGCTGATCTACGACGC |
| | TAGCAACAGATACACCGGCATCCCCGCTAGATTCAGCGGCAGCGGCAGCGGCA |
| | CCGAGTTCACCCTGACCATCAGCAGCCTGCAGAGCGAGGACTTCGCTGTGTAC |
| | TACTGCCAACAAGACTACACAAGCCCCTGGACCTTCGGGCAAGGCACCAAACT |
| | GGAAATTAAGAGCGGAGGTGGTGGGCAAGTGCAGCTGGTGCAGAGCGGCGCCG |
| | AGGTGAAGAAGCCCGGCGCTAGCGTGAAAGTGAGCTGCAAGGCCTCCGGATAC |
| | ACCTTCACCAACTACGGCATGAACTGGGTGAGACAAGCCCCCGGCCAAGGCCT |
| | GGAGTGGATGGGCTGGATCAACACCTACACCGGCGAGCCCACCTACGCCGACG |
| | ACTTCAAGGGCAGAGTGACCATGACCACCGACACAAGCACAAGCACCGCCTAC |
| | ATGGAGCTGAGAAGCCTGAGAAGCGACGACACCGCCGTGTACTATTGCGCGCG |
| | GATCGGCGACAGCAGCCCTAGCGACTACTGGGGCCAAGGCACCCTGGTTACCG |
| | TGAGCAGCGGCGGCTGCCCCCCCTGCCCACCCTGCTGA |
| | (SEQ ID NO: 59) |
| IAB57C2-10 | ATGGAGACCGACACCCTGCTGCTGTGGGTGCTCCTGCTGTGGGTTCCGGGATC |
| | CACCGGCCAAGTGCAGCTGGTGCAGAGCGGCGCCGAGGTGAAGAAGCCCGGCG |
| | CTAGCGTGAAGGTAAGCTGCAAGGCTAGCGGGTACACCTTCACCAACTACGGC |
| | ATGAACTGGGTGAGACAAGCCCCCGGCCAAGGCCTGGAGTGGATGGGCTGGAT |
| | CAACACCTACACCGGCGAGCCCACCTACGCCGACGACTTCAAGGGCAGAGTGA |
| | CCATGACCACCGACACAAGCACAAGCACCGCCTACATGGAGCTGAGAAGCCTG |
| | AGAAGCGACGACACCGCTGTGTACTACTGCGCTCGCATCGGCGACAGCAGCCC |
| | TAGCGACTACTGGGGGCAAGGCACCCTTGTCACCGTGAGCTCGAGCGGTGGCG |
| | GCGGTGAGATCGTGATGACACAGAGCCCCGCTACACTGAGCGTGAGCCCCGGC |
| | GAGAGAGCCACGCTGAGCTGCAAAGCCTCTCAGAGCGTGAGCAACGACGTGGT |
| | GTGGTATCAGCAGAAGCCCGGCCAAGCCCCTAGACTGCTGATCTACGACGCTA |
| | GCAACAGATACACCGGCATCCCCGCTAGATTCAGCGGCAGCGGCAGCGGCACC |
| | GAGTTCACCCTGACCATCAGCAGCCTGCAGAGCGAGGACTTCGCCGTGTACTA |
| | TTGCCAACAAGACTACACAAGCCCCTGGACCTTCGGCCAAGGAACCAAGCTGG |
| | AGATCAAGGGCGGCTGCCCCCCCTGCCCACCCTGCTGA |
| | (SEQ ID NO: 60) |
| IAB57C3-11 | ATGGAGACCGACACCCTGCTGCTGTGGGTGCTGCTCCTGTGGGTGCCCGGTAG |
| | CACCGGCGAGATCGTGATGACACAGAGCCCCGCGACCCTCAGCGTCAGTCCCG |
| | CCGAGAGAGCCACGCTGAGCTGCAAGGCATCTCAGAGCGTGAGCAACGACGTG |
| | GTGTGGTATCAGCAGAAGCCCGGCCAAGCCCCTAGACTGCTGATCTACGACGC |
| | TAGCAACAGATACACCGGCATCCCCGCTAGATTCAGCGGCAGCGGCAGCGGCA |
| | CCGAGTTCACCCTGACCATCAGCAGCCTGCAGAGCGAGGACTTCGCTGTGTAC |
| | TACTGCCAACAAGACTACACAAGCCCCTGGACCTTCGGGCAAGGCACCAAACT |
| | GGAAATTAAGGGCGGAGGTAGCGGAGGTGGTGGGCAAGTGCAGCTGGTGCAGA |
| | GCGGCGCCGAGGTGAAGAAGCCCGGCGCTAGCGTGAAAGTGAGCTGCAAGGCC |
| | TCCGGATACACCTTCACCAACTACGGCATGAACTGGGTGAGACAAGCCCCCGG |
| | CCAAGGCCTGGAGTGGATGGGCTGGATCAACACCTACACCGGCGAGCCCACCT |
| | ACGCCGACGACTTCAAGGGCAGAGTGACCATGACCACCGACACAAGCACAAGC |
| | ACCGCCTACATGGAGCTGAGAAGCCTGAGAAGCGACGACACCGCCGTGTACTA |
| | TTGCGCGCGGATCGGCGACAGCAGCCCTAGCGACTACTGGGGCCAAGGCACCC |
| | TGGTTACCGTGAGCAGCGGCGGCTGCCCCCCCTGCCCACCCTGCTGA |
| | (SEQ ID NO: 61) |
| IAB57C4-12 | ATGGAGACCGACACCCTGCTGCTGTGGGTGCTCCTACTGTGGGTGCCCGGCAG |
| | CACCGGCGAGATCGTGATGACACAGAGCCCCGCGACACTCAGCGTGAGTCCCG |
| | GCGAGAGAGCGACGCTCAGCTGCAAGGCAAGCCAAAGCGTGAGCAACGACGTG |
| | GTGTGGTATCAGCAGAAGCCCGGCCAAGCCCCTCAACTGCTGATCTACGACGC |
| | TAGCAACAGATACACCGGCATCCCCGCTAGATTCAGCGGCAGCGGCAGCGGCA |
| | CCGAGTTCACCCTGACCATCAGCAGCCTGCAGAGCGAGGACTTCGCCGTTTAC |
| | TACTGTCAACAAGACTACACAAGCCCCTGGACCTTCGGCCAAGGCACCAAGCT |
| | GGAAATTAAGGGCAGCACCTCTGGAGGCGGGTCGGGCGGAGGATCGGGAGGCG |
| | GCGGAAGCAGCCAAGTGCAGCTGGTGCAGAGCGGCGCCGAGGTGAAGAAGCCC |
| | GGCGCTAGCGTGAAGGTATCTTGCAAGGCTAGCGGGTATACCTTCACCAACTA |
| | CGGCATGAACTGGGTGAGACAAGCCCCCGGCCAAGGCCTGGAGTGGATGGGCT |
| | GGATCAACACCTACACCGGCGAGCCCACCTACGCCGACGACTTCAAGGGCAGA |
| | GTGACCATGACCACCGACACAAGCACAAGCACCGCCTACATGGAGCTGAGAAG |
| | CCTGAGAAGCGACGACACCGCTGTGTATTATTGCGCGCGGATCGGCGACAGCA |
| | GCCCTAGCGACTACTGGGGACAAGGCACCCTGGTTACCGTGAGCAGCGAACCC |
| | AAGAGCTCCGACAAGACCCACACGTGTCCCCCTTGCCCTCCTTGTGGCGGAGG |
| | AAGCTCCGGAGGCGGAAGCGGAGGACAGCCTAGGGAGCCCCAGGTGTATACCC |
| | TCCCCCCCTCCAGGGAAGAGATGACCAAGAACCAGGTGAGCCTGACCTGCCTC |
| | GTGAAGGGCTTTTATCCCTCCGATATCGCCGTGGAGTGGGAGAGCAACGGCCA |
| | GCCTGAGAACAATTACAAGACCACCCCCCCTGTGCTGGACTCCGATGGCAGCT |
| | TCTTCCTGTATTCCAAGCTGACCGTCGACAAGTCCAGGTGGCAACAGGGCAAC |
| | GTCTTCAGCTGCAGCGTGATGCACGAGGCCCTGCACAATCACTACACCCAGAA |
| | GTCCCTCTCCCTGAGCCCCGGCTGA |
| | (SEQ ID NO: 62) |

### Cells

In some embodiments, a cell line is provided that expresses at least one of the antigen binding constructs, minibodies, or cys-diabodies, described herein In some embodiments, a mammalian cell line (for example, CHO-K1, ExpiCHO, HEK-293, 293f, Expi293 cell lines) is an expression system to produce the antigen binding constructs, minibodies, cys-diabodies, or other antibodies as described herein. In some embodiments, the antigen binding constructs, minibodies, cys-diabodies, and other antibodies or antibody fragments described herein are non-glycosylated, and a mammalian expression system is not required, as such post-translational modifications are not needed. Thus, in some embodiments, one or more of a wide variety of mammalian or non-mammalian expression systems are used to produce antigen binding constructs, minibodies, and cys-diabodies, described herein including, but not limited to mammalian expression systems (for example, CHO-K1 cells), bacterial expression systems (for example, E. coli, B. subtilis) yeast expression systems (for example, Pichia, S. cerevisiae) or any other known expression system. Other systems can include insect cells and/or plant cells.

In some embodiments, the cell expresses an antigen binding construct, minibody, cys-diabody, VL, VH, CDR, FR, linker, signal peptide, or any combination therein. In some embodiments, the cell expresses an antigen binding construct, minibody, cys-diabody, VL, VH, CDR, FR, linker, signal peptide, or any combination therein, having at least about 70%, 75%, 80%, 85%, 90%, 95%, 96%, 97%, 98% or 99% sequence identity to an antigen binding construct, minibody, cys-diabody, VL, VH, CDR, FR, linker, signal peptide, of any of SEQ ID NO: 1-48. In some embodiments, the cell expresses a minibody that is any of SEQ ID NO: 1-10. In some embodiments, the cell expresses a minibody having at least about 70%, 75%, 80%, 85%, 90%, 95%, 96%, 97%, 98% or 99% sequence identity to a minibody that is any of SEQ ID NO: 1-10. In some embodiments, the cell expresses a cys-diabody that is any of SEQ ID NO: 11-14. In some embodiments, the cell expresses a cys-diabody having at least about 70%, 75%, 80%, 85%, 90%, 95%, 96%, 97%, 98% or 99% sequence identity to a cys-diabody that is any of SEQ ID NO: 11-14. In some embodiments, the cell expresses a LCDR that is any of SEQ ID NO: 15-26. In some embodiments, the expression vector encodes a LCDR having at least about 70%, 75%, 80%, 85%, 90%, 95%, 96%, 97%, 98% or 99% sequence identity to a LCDR that is any of SEQ ID NO: 15-26. In some embodiments, the cell expresses a HCDR that is any of SEQ ID NO: 27-32. In some embodiments, the cell expresses a HCDR having at least about 70%, 75%, 80%, 85%, 90%, 95%, 96%, 97%, 98% or 99% sequence identity to a HCDR that is any of SEQ ID NO: 27-32. In some embodiments, the cell expresses a FR that is any of SEQ ID NO: 33-38. In some embodiments, the cell expresses a FR having at least about 70%, 75%, 80%, 85%, 90%, 95%, 96%, 97%, 98% or 99% sequence identity to a FR that is any of SEQ ID NO: 33-38. In some embodiments, the cell expresses a linker that is any of SEQ ID NO: 39-44. In some embodiments, the cell expresses a linker having at least about 70%, 75%, 80%, 85%, 90%, 95%, 96%, 97%, 98% or 99% sequence identity to a linker that is any of SEQ ID NO: 39-44. In some embodiments, the cell expresses a signal peptide that is any of SEQ ID NO: 45-46. In some embodiments, the cell expresses a signal peptide having at least about 70%, 75%, 80%, 85%, 90%, 95%, 96%, 97%, 98% or 99% sequence identity to a signal peptide that is any of SEQ ID NO: 45-46. In some embodiments, the cell expresses a VH that is SEQ ID NO: 47. In some embodiments, the cell expresses a minibody having at least about 70%, 75%, 80%, 85%, 90%, 95%, 96%, 97%, 98% or 99% sequence identity to a VH that is SEQ ID NO: 47. In some embodiments, the cell expresses a VL that is SEQ ID NO: 48. In some embodiments, the cell expresses a VL having at least about 70%, 75%, 80%, 85%, 90%, 95%, 96%, 97%, 98% or 99% sequence identity to a VL that is any of SEQ ID NO: 48.

### Kits

In some embodiments, kits are provided. In some embodiments, the kit includes an antigen binding construct, minibody, cys-diabody, or any combination thereof, as described herein. In some embodiments, the kit includes a nucleic acid that encodes an antigen binding construct as described herein. In some embodiments, the kit includes a cell line that produces an antigen binding construct as described herein. In some embodiments, the kit includes a detectable marker as described herein. In some embodiments, the kit includes a therapeutic agent as described herein. In some embodiments, the kit includes buffers. In some embodiments, the kit includes positive controls, for example target specific cells, or fragments thereof. In some embodiments, the kit includes negative controls, for example a surface or solution that is substantially free of the target. In some embodiments, the kit includes packaging. In some embodiments, the kit includes instructions. In some embodiments, the kit comprises: an antigen binding construct, minibody, or cys-diabody, of any of the preceding embodiments; and a chelator, wherein the chelator allows incorporation of a detectable marker. In some embodiments, the kit comprises: an antigen binding construct, minibody, or cys-diabody, of any of the preceding embodiments; and a chelator, wherein the chelator allows incorporation of a therapeutic isotope. In some embodiments, the kit comprises: an antigen binding construct, minibody, or cys-diabody, of any of the preceding embodiments; and a linker, wherein the linker allows incorporation of a detectable marker. In some embodiments, the kit comprises: an antigen binding construct, minibody, or cys-diabody, of any of the preceding embodiments; and a linker, wherein the linker allows incorporation of a therapeutic isotope.

Additional embodiments of the present disclosure are provided in the following numbered arrangements.
1. An antigen binding construct that comprises:
   a variable light (V_{L}) domain comprising:
      a LCDR1 that is SEQ ID NO: 15;
      a LCDR2 that is any one of SEQ ID NO: 19, 21, or 23;
      a LCDR3 that is SEQ ID NO: 25; and
   a variable heavy (V_{H}) domain comprising:
      a HCDR1 that is SEQ ID NO: 27;
      a HCDR2 that is SEQ ID NO: 29; and
      a HCDR3 that is SEQ ID NO: 31.
2. A minibody that binds to DLL3, the minibody comprising:
   a single-chain variable fragment (scFv) that binds to DLL3, the scFv comprising a variable light (V_{L}) domain linked to a variable heavy (V_{H}) domain, the V_{L} domain comprising:
      a LCDR1 that is SEQ ID NO: 15;
      a LCDR2 that is any one of SEQ ID NO: 19, 21, or 23;
      a LCDR3 that is SEQ ID NO: 25; and
   the V_{H} domain comprising:
      a HCDR1 that is SEQ ID NO: 27;
      a HCDR2 that is SEQ ID NO: 29;
      a HCDR3 that is SEQ ID NO: 31;
   a hinge-extension domain comprising a hinge region; and
   a IgG C_{H}3 sequence.
3. A cys-diabody that binds to DLL3, the cys-diabody comprising a polypeptide that comprises:
   a single-chain variable fragment (scFv) comprising a variable light (V_{L}) domain linked to a variable heavy (V_{H}) domain; and
   the V_{L} domain comprising:
      a LCDR1 that is SEQ ID NO: 15;
      a LCDR2 that is any one of SEQ ID NO: 19, 21, or 23;
      a LCDR3 that is SEQ ID NO: 25; and
   the V_{H} domain comprising:
      a HCDR1 that is SEQ ID NO: 27;
      a HCDR2 that is SEQ ID NO: 29; and
      a HCDR3 that is SEQ ID NO: 31.
4. The antigen binding construct, minibody, or cys-diabody of any one of the preceding arrangements, wherein the V_{L} domain further comprises a LFR2 of SEQ ID NO: 35 or 37.
5. The antigen binding construct, minibody, or cys-diabody of any one of the preceding arrangements, wherein the V_{L} domain comprises an amino acid sequence having at least 90% identity to a V_{L} domain in SEQ ID NO: 48, and wherein the antigen binding construct, minibody, or cys-diabody comprises a Y50D and/or Y55N mutation (Kabat).
6. The antigen binding construct, minibody, or cys-diabody of any one of the preceding arrangements, wherein the V_{L} domain comprises an amino acid sequence having at least 90% identity to a V_{L} domain in SEQ ID NO: 48, and wherein the antigen binding construct, minibody, or cys-diabody comprises a A43S and/or Q45R mutation (Kabat).
7. The antigen binding construct, minibody, or cys-diabody of any one of the preceding arrangements, wherein the V_{L} domain comprises an amino acid sequence having at least 90% identity to a V_{L} domain in SEQ ID NO: 49, and wherein the antigen binding construct, minibody, or cys-diabody comprises a Y50D and/or Y55N mutation (Kabat).
8. The antigen binding construct, minibody, or cys-diabody of any one of the preceding arrangements, wherein the V_{L} domain comprises an amino acid sequence having at least 90% identity to a V_{L} domain in SEQ ID NO: 49, and wherein the antigen binding construct, minibody, or cys-diabody comprises a A43S and/or Q45R mutation (Kabat).
9. The antigen binding construct, minibody, or cys-diabody of any one of the preceding arrangements, wherein the antigen binding construct, minibody, or cys-diabody, further comprises a signal peptide that is SEQ ID NO: 45.
10. The antigen binding construct, minibody, or cys-diabody of any one of the preceding arrangements, wherein the antigen binding construct, minibody, or cys-diabody, comprise a linker that is any one of SEQ ID NO: 39-44.
11. The antigen binding construct or minibody of any one of the preceding arrangements, wherein the C_{H}3 domain is an IgG C_{H}3 domain.
12. The antigen binding construct or minibody of any one of the preceding arrangements, wherein the IgG C_{H}3 domain is an IgG1, IgG2, IgG3, or IgG4 C_{H}3 domain.
13. The antigen binding construct or minibody of any one of the preceding arrangements, wherein the IgG C_{H}3 domain comprises an IgG1 C_{H}3 domain that is any one of SEQ ID NO: 65-68.
14. The antigen binding construct or minibody of any one of the preceding arrangements, wherein the IgG C_{H}3 domain comprises an IgG1 C_{H}3 domain that is any one of SEQ ID NO: 69-71.
15. The antigen binding construct or minibody of any one of the preceding arrangements, wherein the IgG C_{H}3 domain comprises an IgG1 C_{H}3 domain that is any one of SEQ ID NO: 72-82.
16. The antigen binding construct or minibody of any one of the preceding arrangements, wherein the IgG C_{H}3 domain comprises an IgG1 C_{H}3 domain that is any one of SEQ ID NO: 83-85.
17. The antigen binding construct of arrangement 1, wherein the antigen binding construct is an antibody.
18. The antigen binding construct, minibody, or cys-diabody of any one of the preceding arrangement, wherein the antigen binding construct, minibody, or cys-diabody, binds specifically to DLL3.
19. The antigen binding construct, minibody, or cys-diabody of any one of the preceding arrangements, further comprising a detectable marker.
20. The antigen binding construct, minibody, or cys-diabody of any one of the preceding arrangements, wherein the detectable marker is a fluorescently detectable marker.
21. The antigen binding construct, minibody, or cys-diabody of any one of the preceding arrangements, wherein the detectable marker comprises a phototherapy compatible dye.
22. The antigen binding construct, minibody, or cys-diabody of any one of the preceding arrangements, wherein the detectable marker is comprises boron.
23. The antigen binding construct, minibody, or cys-diabody of any one of the preceding arrangements, wherein the detectable marker is compatible for use with Boron Neutron Capture therapy (BNCT).
24. The antigen binding construct, minibody, or cys-diabody of any one of the preceding arrangements, wherein the detectable marker is a radiolabel.
25. The antigen binding construct, minibody, or cys-diabody of any one of the preceding arrangements, wherein the detectable marker is an alpha-emitter radiolabel.
26. The antigen binding construct, minibody, or cys-diabody of any one of the preceding arrangements, wherein the detectable marker is a beta-emitter radiolabel.
27. The antigen binding construct, minibody, or cys-diabody of any one of the preceding arrangements, wherein the detectable marker is an positron-emitter radiolabel.
28. The antigen binding construct, minibody, or cys-diabody of any one of the preceding arrangements, wherein the detectable marker comprises Lutetium-177.
29. The antigen binding construct, minibody, or cys-diabody of any one of the preceding arrangements, wherein the detectable marker is a gamma-emitter radiolabel.
30. The antigen binding construct, minibody, or cys-diabody of any one of the preceding arrangements, wherein the detectable marker is suitable for use with Auger electron spectroscopy.
31. The antigen binding construct, minibody, or cys-diabody of any one of the preceding arrangements, wherein the detectable marker comprises an isotope.
32. The antigen binding construct, minibody, or cys-diabody of any one of the preceding arrangements, wherein the detectable marker comprises a bioluminescent compound.
33. The antigen binding construct, minibody, or cys-diabody of any one of the preceding arrangements, wherein the detectable marker comprises a chemiluminescent compound.
34. The antigen binding construct, minibody, or cys-diabody of any one of the preceding arrangements, wherein the detectable marker comprises an enzyme.
35. The antigen binding construct, minibody, or cys-diabody of any one of the preceding arrangements, wherein the detectable marker comprises a metal chelator.
36. The antigen binding construct, minibody, or cys-diabody of any one of the preceding arrangements, further comprising a therapeutic agent.
37. The antigen binding construct, minibody, or cys-diabody of any one of the preceding arrangements, wherein the therapeutic agent comprises a therapeutic isotope or ion.
38. The antigen binding construct, minibody, or cys-diabody of any one of the preceding arrangements, wherein the therapeutic agent is a radiolabel.
39. The antigen binding construct, minibody, or cys-diabody of any one of the preceding arrangements, wherein the therapeutic agent is an alpha-emitter radiolabel.
40. The antigen binding construct, minibody, or cys-diabody of any one of the preceding arrangements, wherein the therapeutic agent is a beta-emitter radiolabel.
41. The antigen binding construct, minibody, or cys-diabody of any one of the preceding arrangements, wherein the therapeutic agent is an positron-emitter radiolabel.
42. The antigen binding construct, minibody, or cys-diabody of any one of the preceding arrangements, wherein the therapeutic agent is a gamma-emitter radiolabel.
43. The antigen binding construct, minibody, or cys-diabody of any one of the preceding arrangements, wherein the therapeutic agent comprises Lutetium-177.
44. The antigen binding construct, minibody, or cys-diabody of any one of the preceding arrangements, wherein the therapeutic agent comprises a phototherapy compatible dye.
45. The antigen binding construct, minibody, or cys-diabody of any one of the preceding arrangements, wherein the therapeutic agent comprises boron.
46. The antigen binding construct, minibody, or cys-diabody of any one of the preceding arrangements, wherein the therapeutic agent is compatible for use with Boron Neutron Capture therapy (BNCT).
47. The antigen binding construct, minibody, or cys-diabody of any one of the preceding arrangements, wherein the therapeutic agent and/or detectable marker comprise a toxic payload.
48. The antigen binding construct, minibody, or cys-diabody of any one of the preceding arrangements, wherein the antigen binding construct, minibody, or cys-diabody, is bispecific.
49. The antigen binding construct, minibody, or cys-diabody of any one of the preceding arrangements, wherein the antigen binding construct, minibody, or cys-diabody comprises a monovalent scFv.
50. The antigen binding construct, minibody, or cys-diabody of any one of the preceding arrangements, from N terminus to C terminus of the polypeptide is V_{L}, V_{H}.
51. The antigen binding construct, minibody, or cys-diabody of any one of the preceding arrangements, wherein the order of the variable domains, from N terminus to C terminus of the polypeptide is V_{H}, V_{L}.
52. The antigen binding construct, minibody, or cys-diabody of any one of the preceding arrangements, wherein the minibody is a humanized antigen binding construct, minibody, or cys-diabody.
53. The antigen binding construct, minibody, or cys-diabody of any one of the preceding arrangements, wherein the humanized antigen binding construct, minibody, or cys-diabody comprises:
   a LCDR1 that is SEQ ID NO: 15;
   a LCDR2 that is any one of SEQ ID NO: 19, 21, or 23;
   a LCDR3 that is SEQ ID NO: 25;
   a HCDR1 that is SEQ ID NO: 27;
   a HCDR2 that is SEQ ID NO: 29; and
   a HCDR3 that is SEQ ID NO: 31.
54. The antigen binding construct, minibody, or cys-diabody of any one of the preceding arrangements, wherein the antigen binding construct, minibody, or cys-diabody, has increased expression yield in mammalian cells as compared to an antigen binding construct, minibody, or cys-diabody comprising SEQ ID NO: 1.
55. The antigen binding construct, minibody, or cys-diabody of any one of arrangements 1-53, wherein the antigen binding construct, minibody, or cys-diabody, has between about 75% and 300%, 75% 200%, 75% and 150%, or 75% and 100% expression yield as compared to an antigen binding construct, minibody, or cys-diabody comprising SEQ ID NO: 1.
56. The antigen binding construct, minibody, or cys-diabody of any one of arrangements 1-53, wherein the antigen binding construct, minibody, or cys-diabody, has about 75%, 80%, 85%, 90%, 95%, 100%, 105%, 110%, 115%, 120%, 125%, 150%, 175%, 200%, 250%, or 300% expression yield as compared to an antigen binding construct, minibody, or cys-diabody comprising SEQ ID NO: 1.
57. The antigen binding construct, minibody, or cys-diabody of any one of the preceding arrangements, wherein the antigen binding construct, minibody, or cys-diabody, accumulates to a detectable level in a subject's blood, liver, kidney, spleen, lungs, muscle, bone, heart, stomach, small intestine, large intestine, pancreas, tumor, or any combination therein.
58. The antigen binding construct, minibody, or cys-diabody of arrangement 57, wherein the subject is a mammal.
59. The antigen binding construct, minibody, or cys-diabody of arrangement 57 or 58, wherein the antigen binding construct, minibody, or cys-diabody is detectable at up to about 0.1%, 0.5%, 1%, 2%, 3%, 4%, 5%, 6%, 7%, 8%, 9%, 10%, 15%, 20%, 25%, 30%, or 35% ID/g.
60. The antigen binding construct, minibody, or cys-diabody of arrangement 57 or 58, wherein the antigen binding construct, minibody, or cys-diabody is detectable at between about 0.1% and 35%, 0.1% and 30%, 1% and 35%, 1% and 30%, 5% and 35%, or 5% and 30% ID/g.
61. The antigen binding construct, minibody, or cys-diabody of any one of the preceding arrangements, wherein the antigen binding construct, minibody, or cys-diabody, comprises a LFR2 of a LFR2 of SEQ ID NO: 35 or 37; and wherein the antigen binding construct, minibody, or cys-diabody has improved biodistribution as compared to an antigen binding construct, minibody, or cys-diabody comprising an LFR2 that is a LFR2 of SEQ ID NO: 33.
62. The antigen binding construct, minibody, or cys-diabody of any one of the preceding arrangements, wherein the antigen binding construct, minibody, or cys-diabody, has a KD of less than about 1×10⁻¹⁰ M.
63. The antigen binding construct, minibody, or cys-diabody of any one of the preceding arrangements, wherein the antigen binding construct, minibody, or cys-diabody, has a KD of less than about 1×10⁻¹² M.
64. The antigen binding construct, minibody, or cys-diabody of any one of the preceding arrangements, wherein the antigen binding construct, minibody, or cys-diabody, has an EC₅₀ of about 0.001, 0.005, 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, or 0.20 nM.
65. The antigen binding construct, minibody, or cys-diabody of any one of the preceding arrangements, wherein the antigen binding construct, minibody, or cys-diabody, has an EC₅₀ of between about 0.001 and 0.20 nM, 0.001 and 0.17 nM, 0.001 and 0.12 nM, 0.01 and 0.20 nM, 0.01 and 0.17 nM, 0.01 and 0.12 nM, 0.05 and 0.20 nM, 0.05 and 0.17 nM. 0.05 and 0.15 nM, 0.05 and 0.12 nM, 0.08 and 0.20 nM, 0.08 and 0.17 nM, or 0.08 and 0.12 nM.
66. The antigen binding construct, minibody, or cys-diabody of any one of the preceding arrangements, wherein the antigen binding construct, minibody, or cys-diabody, has an EC₅₀ of up to about 10 nM.
67. A nucleic acid encoding an antigen binding construct, minibody, or cys-diabody, of any of the preceding arrangements.
68. A cell line producing an antigen binding construct, minibody, or cys-diabody, of any of the preceding arrangements.
69. The nucleic acid of any of the preceding arrangements, wherein the nucleic acid is any one of SEQ ID NO: 51-65.
70. A cell line producing an antigen binding construct, minibody, or cys-diabody, of any of the preceding arrangements.
71. A kit comprising:
   an antigen binding construct, minibody, or cys-diabody, of any of the preceding arrangements; and
   a detectable marker.
72. A kit comprising: an antigen binding construct, minibody, or cys-diabody, of any of the preceding arrangements; and a chelator, wherein the chelator allows incorporation of a detectable marker.
73. A kit comprising: an antigen binding construct, minibody, or cys-diabody, of any of the preceding arrangements; and a chelator, wherein the chelator allows incorporation of a therapeutic isotope.
74. A kit comprising: an antigen binding construct, minibody, or cys-diabody, of any of the preceding arrangements; and a linker, wherein the linker allows incorporation of a detectable marker.
75. A kit comprising: an antigen binding construct, minibody, or cys-diabody, of any of the preceding arrangements; and a linker, wherein the linker allows incorporation of a therapeutic isotope.
76. A kit comprising: an antigen binding construct, minibody, or cys-diabody, of any of the preceding arrangements; and a detectable marker.
77. A method of detecting a presence or absence of a DLL3, the method comprising:
   applying the antigen binding construct, minibody, or cys-diabody, of any of the preceding arrangements to a sample; and
   detecting a presence or an absence of the antigen binding construct, thereby detecting a presence or absence of a DLL3.
78. The method of arrangement 77, wherein the antigen binding construct, minibody, or cys-diabody, is conjugated to a detectable marker.
79. The method of arrangement 77 or 78, wherein applying the antigen binding construct, minibody, or cys-diabody, comprises administering the antigen binding construct to a subject.
80. The method of any one of arrangements 77-79, wherein detecting binding or absence of binding of the antigen binding construct, minibody, or cys-diabody, to DLL3 comprises at least one of positron emission tomography or single-photon emission computed tomography.
81. The method of any one of arrangements 77-80, the method further comprising applying a secondary antigen binding construct to the sample, wherein the secondary antigen binding construct binds specifically to the antigen binding construct.
82. The method of any one of arrangements 77-81, wherein the antigen binding construct, minibody, or cys-diabody, is incubated with the sample for no more than 20 hours.
83. The method of any one of arrangements 77-82, wherein the antigen binding construct, minibody, or cys-diabody, is incubated with the sample for no more than 6 hours.
84. The method of any one of arrangements 77-83, wherein the antigen binding construct, minibody, or cys-diabody, is administered to a host, and wherein a first quantity of antigen binding construct, minibody, or cys-diabody, thereof is unbound to DLL3, and a second quantity of antigen binding construct, minibody, or cys-diabody, is bound to DLL3, wherein at least about 80% of the first quantity of antigen binding construct, minibody, or cys-diabody, is eliminated in no more than 12 hours.
85. A method of targeting a therapeutic agent to DLL3, the method comprising administering to a subject an antigen binding construct, minibody, or cys-diabody, of any one of the preceding arrangements, wherein the antigen binding construct is conjugated to a therapeutic agent.
86. A therapeutic composition targeting DLL3, wherein the therapeutic composition comprises:
   an antigen binding construct that comprises:
      a variable light (V_{L}) domain comprising:
      a LCDR1 that is SEQ ID NO: 15;
      a LCDR2 that is any one of SEQ ID NO: 19, 21, or 23;
      a LCDR3 that is SEQ ID NO: 25; and
      a variable heavy (V_{H}) domain comprising:
      a HCDR1 of a HCDR1 that is SEQ ID NO: 27;
      a HCDR2 of HCDR2 that is SEQ ID NO: 29;
      a HCDR3 of HCDR3 that is SEQ ID NO: 31; and
   a therapeutic agent, toxic payload, and/or a detectable marker.
87. A therapeutic composition targeting DLL3, wherein the therapeutic composition comprises:
   a minibody that binds to DLL3, the minibody comprising:
      a single-chain variable fragment (scFv) that binds to DLL3, the scFv comprising a variable light (V_{L}) domain linked to a variable heavy (V_{H}) domain, the V_{L} domain comprising:
      a LCDR1 that is SEQ ID NO: 15;
      a LCDR2 that is any one of SEQ ID NO: 19, 21, or 23;
      a LCDR3 that is SEQ ID NO: 25 and
      the V_{H} domain comprising:
      a HCDR1 that is SEQ ID NO: 27;
      a HCDR2 that is SEQ ID NO: 29;
      a HCDR3 that is SEQ ID NO: 31;
   a hinge-extension domain comprising a IgG1 hinge region;
   a IgG C_{H}3 sequence; and
   a therapeutic agent, toxic payload, and/or a detectable marker.
88. A therapeutic composition targeting DLL3, wherein the therapeutic composition comprises:
   a cys-diabody that binds to DLL3, the cys-diabody comprising a polypeptide that comprises:
   a single-chain variable fragment (scFv) comprising a variable light (V_{L}) domain linked to a variable heavy (V_{H}) domain;
      the V_{L} domain comprising:
      a LCDR1 that is SEQ ID NO: 15;
      a LCDR2 that is SEQ ID NO: 19, 21, or 23;
      a LCDR3 that is SEQ ID NO: 25;
   the V_{H} domain comprising:
      a HCDR1 that is SEQ ID NO: 27;
      a HCDR2 that is SEQ ID NO: 29;
      a HCDR3 that is SEQ ID NO: 31 and;
   a therapeutic agent, toxic payload, and/or a detectable marker.
89. The therapeutic composition of any of the preceding arrangements, wherein the detectable marker is a radiolabel.
90. The therapeutic composition of any of the preceding arrangements, wherein the detectable marker is an alpha-emitter radiolabel.
91. The therapeutic composition of any of the preceding arrangements, wherein the detectable marker is a beta-emitter radiolabel.
92. The therapeutic composition of any of the preceding arrangements, wherein the detectable marker is a positron-emitter radiolabel.
93. The therapeutic composition of any of the preceding arrangements, wherein the detectable marker comprises Lutetium-177.
94. The antigen binding construct, minibody, or cys-diabody of any one of the preceding arrangements, or the composition of any one of the preceding arrangements, or the kit of any one of the preceding arrangements, or the method of any one of the preceding arrangements, wherein the therapeutic agent comprises Terbium-149, Terbium-161, or Lead-212.
95. The antigen binding construct of any one of the preceding arrangements, or the composition of any one of the preceding arrangements, or the kit of any one of the preceding arrangements, or the method of any one of the preceding arrangements, wherein the antigen binding construct is an scFv-Fc comprising:
   a single-chain variable fragment (scFv) that binds to DLL3, the scFv comprising the variable light (V_{L}) domain linked to the variable heavy (V_{H}) domain;
   a hinge domain; and
   an Fc region.
96. An antigen binding construct that binds DLL3, wherein the antigen binding construct is a Nanobody®-Fc that binds DLL3 and comprises a variable heavy (V_{H}) domain linked to a Fc region via a hinge domain.
97. The antigen binding construct of arrangement 96, wherein the variable heavy (V_{H}) domain comprises:
   a HCDR1 that is SEQ ID NO: 27;
   a HCDR2 that is SEQ ID NO: 29; and
   a HCDR3 that is SEQ ID NO: 31.

Embodiments of the present disclosure are further defined in the following Examples. It should be understood that these Examples are given by way of illustration only. From the above discussion and these Examples, one skilled in the art can ascertain the essential characteristics of the present disclosure, and without departing from the spirit and scope thereof, can make various changes and modifications of the embodiments of the disclosure to adapt it to various usages and conditions. Thus, various modifications of the embodiments of the disclosure, in addition to those shown and described herein, will be apparent to those skilled in the art from the foregoing description. Such modifications are also intended to fall within the scope of the appended claims. The disclosure of each reference set forth herein is incorporated herein by reference in its entirety, and for the disclosure referenced herein.

### EXAMPLES

### Example 1: Isolation and Characterization of Novel Antigen Binding Constructs

The isolation of a series of novel antigen binding constructs as described herein can also be performed using any standard methodology known to one skilled in the art.

As disclosed herein, a series of antigen binding constructs specific against DLL3 were designed and isolated (SEQ ID NOS: 1-46). Each antigen binding construct was either human or murine, and either an antibody, a minibody, or a cys-diabody. In one example, the variable region from a parental antibody was converted to a scFv and engrafted in a minibody or cys-diabody scaffold. In another example, the variable region from a parental antibody tested for immunogenic epitopes through in-silico methods known in the art, converted to a scFv and engrafted in a minibody or cys-diabody scaffold. Furthermore, each construct comprised a heavy chain and light chain, with a HCDR1 of SEQ ID NO: 27, a HCDR2 of SEQ ID NO: 29, a HCDR3 of SEQ ID NO:31, a LCDR1 of SEQ ID NO: 15, a LCDR2 of SEQ ID NO: 19, 21, or 23, and a LCDR3 of SEQ ID NO: 25. Each antigen binding construct varied in sequence; for example, some contained a serine at position 43 of the V_{L}, an arginine at position 45 of the V_{L}, an aspartic acid at position 50 of the V_{L}, and/or an asparagine at position 55 of the V_{L} (Kabat). The sequence variations for each antigen binding construct were chosen for their improved characteristics over antigen binding constructs without the variation. For example, variations in the antigen binding construct resulted in improved antigen binding kinetics as illustrated by a flattening, or decrease, of the disassociation constant (K_{off}).

These antigen binding constructs where then transfected into Expi293™ cell-line using a liposome transfection protocol (Thermo Exp293 kit). The vector contained a cleavable signal peptide of SEQ ID NO: 45. In other alternatives, the sequence of the antigen binding constructs can be inserted into any cell line using any conventional method, such as electroporation.

Expression of the constructs was measured compared to parental minibodies IAB57M1-1 and IAB57M2-2. As shown in FIG. 7, the novel antigen binding constructs disclosed herein had up to 4-fold greater expression yield in Expi293™ mammalian cells compared to the parental minibodies.

Binding kinetics of the novel antigen binding constructs were also determined by biolayer interferometry (TABLE 9) (FIG. 9).

TABLE 9
Binding kinetics of antigen binding constructs

| Construct | KD (M) | Ka (1/Ms) | Kd (1/s) |
|---|---|---|---|
| IAB57M1-3 | 4.00E−9 | 5.988E+5 | 2.39E−3 |
| IAB57M2-4 | 4.84E−9 | 5.81E+5 | 2.81E−3 |
| IAB57M2-6 | <1E−12 | 7.85E+5 | <1E−7 |
| IAB57M1-1 | 3.33E−9 | 8.38E+5 | 2.79E−3 |
| IAB57M2-2 | 3.07E−9 | 6.98E+5 | 2.14E−3 |

As disclosed herein, the half-maximal effective concentration (EC₅₀) was also assessed. EC₅₀ was determined (Table 10) (FIG. 8). The novel antigen binding constructs disclosed herein had a high affinity for tumor cells in SHP-77 and NCI-H82 cell lines, with EC₅₀ between 0.01-0.1 nM (FIG. 10).

TABLE 10
EC₅₀ of antigen binding constructs

| Construct | EC₅₀ (nM) |
|---|---|
| IAB57M1-3 | 0.1089 |
| IAB57M1-7 | 0.1113 |
| IAB57M2-4 | 0.1179 |
| IAB57M2-6 | 0.1614 |
| IAB57M1-5 | 0.1250 |
| IAB57M2-2 | 0.1197 |
| IAB57M1-1 | 0.09457 |
| IAB57M2-8 | 0.1231 |

Binding affinity of the novel antigen binding constructs to a purified human/cynomolgus/dog/mouse/rat or minipig DLL3 antigen was assessed using ELISA. For this assay, a 96 well ELISA plate was coated with the DLL3 antigen diluted to a concentration of 2 mg/mL in coating buffer (0.05M sodium carbonate-bicarbonate buffer, pH 9.6). 100 μl of this coating solution was added to each well in the plate and incubate at 4° C. overnight. The plate was washed 3 times with 300 μl per well of washing buffer (PBS with 0.05% Tween-20). After the last wash, the plate was blocked by adding to each well 200 μl of blocking buffer (PBS with 1% BSA) warmed to room temperature. The plate was then incubated at room temperature for 1 hour under gentle agitation. After blocking, the plate was washed 3 times with 300 μl per well of washing buffer. Dilution samples for each minibody were created by making 2.5× dilutions from a starting sample at 100 ng/mL, or 250 ng/mL, or 1000 ng/mL or 3000 ng/mL in 100 μL sample buffer (PBS with 1% BSA). The dilution samples were transferred to the assay plate and incubated at room temperature for 1 hour under gentle agitation. The plate was then washed 3 times with 300 μl per well of washing buffer.

Detection was performed by adding 100 μl of HRP labeled detection secondary antibody at an appropriate dilution and incubating the plate at room temperature for 1 hour under gentle agitation. The plate was then washed 3 times with 300 μl per well of washing buffer. For color development, 100 μl of TMB substrate regent, pre-warmed to room temperature, was added to the plate and incubated in the dark at room temperature for 15 min. The reaction was then stopped by adding 100 μl of 650 nm stop solution to the plate and mixed for 1 min at room temperature under gentle agitation. The plate is recorded by reading OD at 650 nm in a plate reader within 20 min after terminating the reaction.

As disclosed herein, the binding kinetics of the novel antigen binding constructs to a purified human/cynomolgus/dog/mouse/rat or minipig DLL3 antigen was assessed using biolayer interferometry (TABLE 9) (FIG. 9). The his-tagged-DLL3 antigen was diluted in phosphate buffer saline (PBS) at 10 μg/mL and loaded on a nickel-NTA tip until saturation. The on-rate (Kon) and off-rate (K_{off}), and the dissociation constant K_{D}, where recorded on Octet BLItz instrument. Dilution samples of the antigen binding construct were prepared in PBS at concentrations between 100 nM and 0.1 nM, by 2.5× dilutions and binding curves recorded at each concentration. Off-rate curves were recorded by immersion of the tip in PBS.

As disclosed herein, the binding of constructs to cellular targets was assessed using flow cytometry. Stock of 2.5× serial dilution of minibodies were created starting from a concentration of 100 nM in 180 μl FACS buffer (PBS 2% FBS). Cells were added to a Corning V-bottom polypropylene cell culture plate at 200,000 cells/well. The cells were centrifuged at 100 rpm for 3 minutes and the supernatant discarded. The minibody dilution series were transferred to the plate containing cell pellets and incubated for 1 hr at 4° C. The plate was then washed 3 times with FACS buffer at 200 μl/well, discarding supernatant after each wash by centrifugation at 1000 rpm. 50 μl/well of detection antibody R&D AlexaFluor-647 Goat anti Human (H+L) was added at a 1:100 dilution in FACS buffer and incubated at 4° C. 1 hr. The plate was washed 3 times with FACS buffer at 200 μl/well, discarding supernatant after each wash by centrifuging at 1000 rpm. Cells were then fixed by adding 100 μl of 1% paraformaldehyde in PBS and incubating at room temperature for 15 minutes. 100 μl of PBS were added to each well and the plate was analyzed by flow cytometry using the allophycocyanin fluorescence setting.

The humanized V_{L} in IAB57M1-3 used the germline sequence of IGVK3-15*01. The V_{L} has a framework region 2 (FR2) sequence (WYQQKPGQAPRLLI (SEQ ID NO:70) (according to North)) that includes a cluster of two arginines at positions 39 and 45 (IMGT numbering). Molecular modeling of the surface charges using an Adaptive Poisson-Boltzmann Solver identified a localized patch of surface-exposed positive charges formed by the two arginines. Without being bound by theory, it is hypothesized that this localized positively charged patch may contribute to accumulation of minibodies in the kidney leading to overly rapid clearance of the minibody and potential kidney injury.

To test this, R45 was substituted with a glutamine (Q), as shown in FIG. 1. Thus, KPGQAPR (SEQ ID NO: 95) in V_{L} FR2 of IAB57M1-3 was altered to KPGQAPQ (SEQ ID NO: 111) to make IAB57M1-13. IAB57M1-14 was made by further substituting the alanine (A) at position 43 with serine (S), as shown in FIG. 1. Thus, KPGQAPR (SEQ ID NO: 95) in V_{L} FR2 of IAB57M1-3 was further altered to KPGQSPQ (SEQ ID NO: 110) in IAB57M1-14.

The in vivo biodistribution of 89Zr-desferoxamine-IAB57M1-3 in NCr nude mice bearing SHP77 and NCI-H82 tumors was assessed (FIG. 11). The in vivo biodistribution of 89-Zr-Df-IAB57M1-3, 89-Zr-Df-IAB57M1-13, and 89-Zr-Df-IAB57M1-14 were also assessed (FIG. 12). In both cases, the M1-3 antigen binding constructs were relatively highly distributed to the liver and kidney. In contrast, the M1-13 and M1-14 displayed improved biodistribution as indicated by reduced accumulation of these minibodies in the kidney. The antigen binding constructs were also distributed in vivo to the blood, spleen, lungs, muscles, bones, heart, stomach, large intestine, small intestine, pancreas, and tumors.

### Example 2: Use of the Antigen Binding Constructs for Treatment of a Subject in Need Thereof

The methods of utilizing the novel antigen binding constructs described herein as a medicament to a subject in need thereof may confer a benefit to the subject.

As disclosed herein, a pharmaceutical composition is made comprising a novel antigen binding construct against DLL3, alone or in combination with a chemotherapy, or an immuno-oncology drug, and a pharmaceutically acceptable carrier. In other alternatives, the dosage is a radioactive dose. In other alternatives, the dosage is not fixed. In other alternatives, the dosage is provided as fractionated doses. In other alternatives, the dosage is measured in mCi or MBq. In other alternatives, the composition can have more than one antigen binding constructs. In other alternatives, the antigen binding construct can be an antibody, minibody, cys-diabody, or another combination thereof. In other alternatives, the antigen binding construct may be present in the composition at any pharmaceutically effective concentration, such as about 0.01 mg/kg to about 25 mg/kg. In other alternatives, the immuno-oncology drug may be omitted from the composition. In other alternatives, the immuno-oncology drug may be replaced by one or more small molecule, therapeutic, or antigen binding construct effective in treating a disease, such as an antibody, a chemotherapy drug, a DNA repair inhibitor, an alkylating agent, a metabolic inhibitor, a radiosensitizer agent, an anti-tumor antibiotic, a topoisomerase inhibitor, a mitotic inhibitor, a nitrosourea, a corticosteroid, an anti-angiogenic, an apoptosis inducer, an anti-microtubule agent, a vinca alkaloid, a taxane, an anthracycline, an anti-androgen, a VEGF pathway inhibitor, a VEGF pathway inhibitor, a MAPK/Ras/Raf pathway inhibitor, and an EGFR pathway inhibitor.

The compositions of the invention may be employed in combination therapies with other agents to achieve an improved therapeutic outcome for the subject. The other agents which may be employed in combination include therapeutic agents, immunotherapeutic agents, chemotherapeutic agents, radioprotective agents and kidney protecting agents. Immunotherapeutic agents useful in combination with the invention include Atezolizumab (Tecentriq), Avelumab (Bavencio), Dostarlizumab (Jemperli), Durvalumab (Imfinzi), Ipilimumab (Yervoy), Nivolumab (Opdivo), Pembrolizumab (Keytruda), and other immunotherapeutic agents in clinical development.

As disclosed herein, the composition is administered to a human subject at a pharmaceutically acceptable dose for treating SCLC. In other alternatives, the disease may be other neuroendocrine tumors such as, neuroendocrine prostate cancer, glioblastoma, fibrosis, cancer, a tumor, a solid tumor, an autoimmune disease, cardiovascular disease, metabolic disease, bone cancer, bone sarcoma, breast cancer, carcinoid, cervical cancer, colon cancer, colorectal cancer, endometrial carcinoma, epithelial ovarian cancer, esophageal cancer, gastric cancer, gastrointestinal cancer, glioma, head and neck cancer, hepatocellular cancer, kidney cancer, leukemia, liver cancer, lung cancer, lymphoma, medullary thyroid carcinoma, melanoma, non-small cell lung cancer, osteosarcoma, oral squamous cell carcinoma, oral cancer, ovarian carcinoma, ovarian cancer, pancreatic adenocarcinoma, pancreatic cancer, prostate cancer, rectal cancer, renal cancer, skin cancer, stomach cancer, testis cancer, thyroid cancer, urothelial cancer, or any other disease associated with changing DLL3 expression. In other alternatives, the composition is administered to a subject for inhibiting or ameliorating the disease.

After administration, the disease is monitored through the imaging of relevant cells, tissues, or organs in the subject using the method outlined in the example below. As seen through the imaging, the cancer cells are damaged following administration of the composition to the subject. In other alternatives, the cancer cell are inhibited, ameliorated, damaged, killed, or has induced apoptosis in response to contact with the composition.

### Example 3: Use of the Antigen Binding Constructs for Diagnostics

The methods of utilizing the novel antigen binding constructs described herein for diagnostics thereof may confer a benefit to a subject suspected of having a disease.

As disclosed herein, the novel antigen binding construct is used for imaging the tissue of a human subject suspected of having cancer. In other alternatives, the antigen binding constructs can be used for imaging a cell, cultured cell line, fraction of tissue, organ, organ sectional, multi-tissue, multi-organ, or whole subject. In other alternatives, the subject can be screened for a tumor, fibrosis, autoimmune disease, cardiovascular disease, or any other disease or abnormality associated with DLL3. In other alternatives, the subject can be any mammal, including mice, rats, dog, minipig and non-human primates.

The antigen binding construct is administered to the epithelial tissue of a subject and screened for binding to DLL3. The subject is evaluated for the presence of DLL3 by methods of in-vivo diagnostic medical imaging such as, positron emission tomography (PET), or Single-photon emission computed tomography (SPECT). The binding of the antigen binding construct to DLL3 indicates a high likelihood for the presence of cancer in the tissue. In other alternatives, the absence of binding of the antigen binding construct to DLL3 indicates a low likelihood for the presence of cancer in the tissue. In other alternatives, the binding of the antigen binding construct to DLL3 is determined by a payload added to the antigen binding construct, such as ¹⁸F, ¹⁸F-FAC, ³²P ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁵Sc, ⁷⁷As, ⁸⁶Y, ⁹⁰Y, ⁸⁹Sr, ⁸⁹Zr, ⁹⁴Tc, ⁹⁴Tc, ⁹⁹mTc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁷Th or ²²⁵Ac. In other alternatives, the binding of the antigen binding construct to DLL3 is determined through a PET scan.

### Example 4: Use of Antigen Binding Constructs for Reducing Uptake in Kidneys

The methods of modifying the antigen binding constructs described herein may confer a benefit of reduced kidney uptake of radiolabeled minibodies in the subject.

As disclose herein, a minibody is made through humanization of a mouse hybridoma. In some embodiments, the humanization of the antibody is based on germlines: IGHV1-69*01/IGHV1-39*01.

Positively charged patch is identified on the germline IGKV1-39*01 which is responsible for accumulating this minibody in the kidney. As disclosed herein, the patch maps to sequence KPGKAPK which contains 3 charged lysine residues. In some embodiments, mutations on the framework 2 of this germline were identified that reduce kidney uptake of radiolabeled minibodies.

As disclosed herein, several human germlines have a pattern of three positively charged residues at framework 2. In some embodiments, the positively charged residue is a lysine (K) and/or arginine (R). In some embodiments, the germline IGKV1-39*01 is modified where the framework 2 is substituted with similar sequence from germlines that do not contain this charge pattern. A non-limiting example for such substitution is framework 2 from IGKV3-20*01 or IGKV2-28*01.

As used herein, the section headings are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose, including the disclosures specifically referenced herein. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings herein.

Although this disclosure has been presented in the context of certain embodiments and examples, those skilled in the art will understand that the present disclosure extends beyond the specifically disclosed embodiments to other alternative embodiments and/or uses of the disclosure and obvious modifications and equivalents thereof. In addition, while several variations of the present disclosure have been shown and described in detail, other modifications, which are within the scope of this disclosure, will be readily apparent to those of skill in the art based upon this disclosure. It is also contemplated that various combinations or sub-combinations of the specific features and aspects of the embodiments may be made and still fall within the scope of the present disclosure. It should be understood that various features and aspects of the disclosed embodiments can be combined with, or substituted for, one another in order to form varying modes or embodiments of the present disclosure. Thus, it is intended that the scope of the present disclosure herein presented should not be limited by the particular disclosed embodiments described above.

It should be understood, however, that this detailed description, while indicating preferred embodiments of the disclosure, is given by way of illustration only, since various changes and modifications within the spirit and scope of the disclosure will become apparent to those skilled in the art.

The terminology used in the description presented herein is not intended to be interpreted in any limited or restrictive manner. Rather, the terminology is simply being utilized in conjunction with a detailed description of embodiments of the systems, methods and related components. Furthermore, embodiments may comprise several novel features, no single one of which is solely responsible for its desirable attributes or is believed to be essential to practicing the disclosures herein described.

1. An antigen binding construct that comprises:
a variable light (V_{L}) domain comprising:
a LCDR1 that is SEQ ID NO: 15;
a LCDR2 that is any one of SEQ ID NO: 19, 21, or 23;
a LCDR3 that is SEQ ID NO: 25; and
a variable heavy (V_{H}) domain comprising:
a HCDR1 that is SEQ ID NO: 27;
a HCDR2 that is SEQ ID NO: 29; and
a HCDR3 that is SEQ ID NO: 31.

2. A minibody that binds to DLL3, the minibody comprising:
a single-chain variable fragment (scFv) that binds to DLL3, the scFv comprising a variable light (V_{L}) domain linked to a variable heavy (V_{H}) domain, the V_{L} domain comprising:
a LCDR1 that is SEQ ID NO: 15;
a LCDR2 that is any one of SEQ ID NO: 19, 21, or 23;
a LCDR3 that is SEQ ID NO: 25; and
the V_{H} domain comprising:
a HCDR1 that is SEQ ID NO: 27;
a HCDR2 that is SEQ ID NO: 29;
a HCDR3 that is SEQ ID NO: 31;
a hinge-extension domain comprising a hinge region; and
a IgG C_{H}3 sequence.

3. A cys-diabody that binds to DLL3, the cys-diabody comprising a polypeptide that comprises:
a single-chain variable fragment (scFv) comprising a variable light (V_{L}) domain linked to a variable heavy (V_{H}) domain; and
the V_{L} domain comprising:
a LCDR1 that is SEQ ID NO: 15;
a LCDR2 that is any one of SEQ ID NO: 19, 21, or 23;
a LCDR3 that is SEQ ID NO: 25; and
the V_{H} domain comprising:
a HCDR1 that is SEQ ID NO: 27;
a HCDR2 that is SEQ ID NO: 29; and
a HCDR3 that is SEQ ID NO: 31.

4. The antigen binding construct of claim 1, wherein the V_{L} domain further comprises a LFR2 of SEQ ID NO: 35 or 37.

5. The antigen binding construct of claim 1, wherein
(a) the V_{L} domain comprises an amino acid sequence having at least 90% identity to a V_{L} domain in SEQ ID NO: 48, and wherein the antigen binding construct comprises a Y50D and/or Y55N mutation (Kabat),
(b) the V_{L} domain comprises an amino acid sequence having at least 90% identity to a V_{L} domain in SEQ ID NO: 48, and wherein the antigen binding construct comprises a A43S and/or Q45R mutation (Kabat),
(c) the V_{L} domain comprises an amino acid sequence having at least 90% identity to a V_{L} domain in SEQ ID NO: 49, and wherein the antigen binding construct comprises a Y50D and/or Y55N mutation (Kabat), or
(d) the V_{L} domain comprises an amino acid sequence having at least 90% identity to a V_{L} domain in SEQ ID NO: 49, and wherein the antigen binding construct comprises a A43S and/or Q45R mutation (Kabat).

6.-8. (canceled)

9. The antigen binding construct of claim 1, wherein the antigen binding construct further comprises a signal peptide that is SEQ ID NO: 45 and/or comprises a linker that is any one of SEQ ID NO: 39-44.

10. (canceled)

11. The antigen binding construct of claim 1, wherein the C_{H}3 domain is an IgG C_{H}3 domain.

12. The antigen binding construct of claim 1, wherein
(a) the IgG C_{H}3 domain is an IgG1, IgG2, IgG3, or IgG4 C_{H}3 domain,
(b) the IgG C_{H}3 domain comprises an IgG1 C_{H}3 domain that is any one of SEQ ID NO: 65-68,
(c) the IgG C_{H}3 domain comprises an IgG1 C_{H}3 domain that is any one of SEQ ID NO: 69-71,
(d) the IgG C_{H}3 domain comprises an IgG1 C_{H}3 domain that is any one of SEQ ID NO: 72-82, or
(e) the IgG C_{H}3 domain comprises an IgG1 C_{H}3 domain that is any one of SEQ ID NO: 83-85.

13-18. (canceled)

19. The antigen binding construct of claim 1, further comprising a detectable marker or a radiolabel.

20.-30. (canceled)

31. The antigen binding construct of claim 19, wherein the detectable marker comprises an isotope, a bioluminescent compound, a chemiluminescent compound, an enzyme, and/or a metal chelator.

32.-35. (canceled)

36. The antigen binding construct of claim 1, further comprising a therapeutic agent.

37. The antigen binding construct of claim 36, wherein the therapeutic agent comprises a therapeutic isotope or ion, a radiolabel, and/or a phototherapy compatible dye.

38.-51. (canceled)

52. The antigen binding construct of claim 1, that is a humanized antigen binding construct.

53. (canceled)

54. The antigen binding construct of claim 1, wherein
(a) the antigen binding construct has increased expression yield in mammalian cells as compared to an antigen binding construct comprising SEQ ID NO: 1,
(b) the antigen binding construct has from 75% to 150% expression yield as compared to an antigen binding construct comprising SEQ ID NO: 1, or
(c) the antigen binding construct has from 100% to 300% expression yield as compared to an antigen binding construct comprising SEQ ID NO: 1.

55.-60. (canceled)

61. The antigen binding construct of claim 1, wherein the antigen binding construct comprises a LFR2 of a LFR2 of SEQ ID NO: 35 or 37; and wherein the antigen binding construct has improved biodistribution as compared to an antigen binding construct, minibody, or cys-diabody comprising an LFR2 that is a LFR2 of SEQ ID NO: 33.

62. The antigen binding construct of claim 1, wherein the antigen binding construct, minibody, or cys-diabody, has a KD of less than about 1×10⁻¹⁰ M or 1×10⁻¹² M.

63. (canceled)

64. The antigen binding construct of claim 1, wherein the antigen binding construct, minibody, or cys-diabody, has an EC₅₀ from 0.001 to 0.20 nM.

65.-70. (canceled)

71. A kit comprising:
an antigen binding construct of claim 1; and
a detectable marker, and/or a detectable marker, and/or a linker, wherein the linker allows incorporation of a detectable marker or a therapeutic isotope.

72. The kit of claim 71, wherein the chelator allows incorporation of a detectable marker, and/or wherein the chelator allows incorporation of a therapeutic isotope.

73.-76. (canceled)

77. A method of detecting a presence or absence of a DLL3, the method comprising:
applying the antigen binding construct of claim 1 to a sample, wherein the antigen binding construct is conjugated to a detectable marker; and
detecting a presence or an absence of the antigen binding construct, thereby detecting a presence or absence of a DLL3.

78. (canceled)

79. (canceled)

80. The method of claim 77, wherein detecting binding or absence of binding of the antigen binding construct to DLL3 comprises at least one of positron emission tomography or single-photon emission computed tomography.

81.-84. (canceled)

85. A method of targeting a therapeutic agent to DLL3, the method comprising administering to a subject an antigen binding construct of claim 1, wherein the antigen binding construct is conjugated to a therapeutic agent.

86. A therapeutic composition targeting DLL3, wherein the therapeutic composition comprises:
the antigen binding construct of claim 1; and
a therapeutic agent, toxic payload, and/or a detectable marker.

87.-94. (canceled)

95. The antigen binding construct of claim 1, wherein the antigen binding construct is an scFv-Fc comprising:
a single-chain variable fragment (scFv) that binds to DLL3, the scFv comprising the variable light (V_{L}) domain linked to the variable heavy (V_{H}) domain;
a hinge domain; and
an Fc region.

96. An antigen binding construct that binds DLL3, wherein the antigen binding construct is a Nanobody®-Fc that binds DLL3 and comprises a variable heavy (V_{H}) domain linked to a Fc region via a hinge domain.

97. (canceled)
